(19) United States
(12) Patent Application Publication
(10) Pub. No.: US 2024/0206329 A1
(43) Pub. Date: Jun. 20, 2024

(54) COMPOUND, ORGANIC PHOTODETECTOR INCLUDING THE COMPOUND, AND ELECTRONIC APPARATUS INCLUDING THE ORGANIC PHOTODETECTOR

(71) Applicants: Samsung Display Co., LTD., Yongin-si (KR); Korea University Research And Business Foundation, Seoul (KR)

(72) Inventors: Hwasook Ryu, Yongin-si (KR); Han Young Woo, Seoul (KR); Seokgyu Yoon, Yongin-si (KR); Daeho Lee, Yongin-si (KR); Hyejin Jung, Yongin-si (KR); Venkata Suman Krishna Jonnadula, Seoul (KR); Ziang Wu, Seoul (KR); Min Hun Jee, Seoul (KR)

(21) Appl. No.: 18/507,984

(22) Filed: Nov. 13, 2023

(30) Foreign Application Priority Data
Nov. 18, 2022 (KR) 10-2022-0155791

(51) Int. Cl. H10K 85/60; C07C 49/683; C07D 333/22; H10K 30/20

(52) U.S. Cl. CPC H10K 85/655; C07C 49/683; C07D 333/22; H10K 30/20; H10K 85/626

(57) ABSTRACT

Provided are a compound represented by one selected from Formulae 1 to 4, an organic photodetector including the compound, and an electronic apparatus including the organic photodetector. Formulae 1 to 4 are shown below, and the variables of Formulae 1 to 4 are as described in the disclosure.

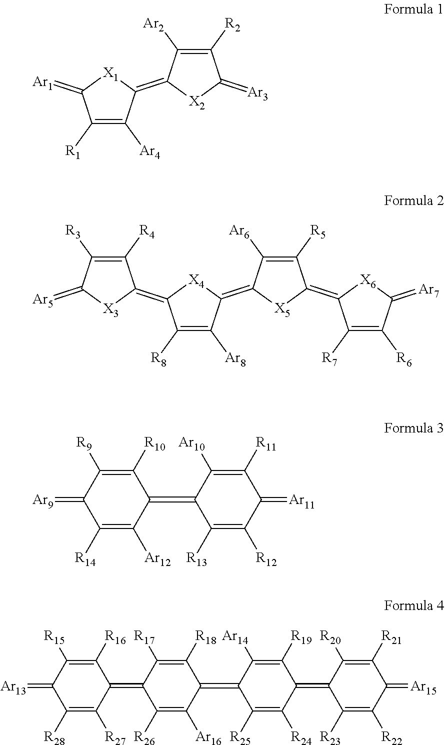

### CROSS-REFERENCE TO RELATED APPLICATION

This application claims priority to and the benefit of Korean Patent Application No. 10-2022-0155791, filed on Nov. 18, 2022, in the Korean Intellectual Property Office, the entire content of which is incorporated by reference herein.

### BACKGROUND

### 1. Field

One or more embodiments of the present disclosure relate to a compound, an organic photodetector including the compound, and an electronic apparatus including the organic photodetector.

### 2. Description of the Related Art

Photoelectric devices are devices that convert light and an electrical signal and include a photodiode and a phototransistor. Photoelectric devices may be applied to an image sensor, a solar cell, an organic light-emitting device, and/or the like.

In the case of silicon, which is mainly used in photodiodes, as the size of pixels decreases, an absorption region may decrease, thereby deteriorating or reducing sensitivity. Accordingly, organic materials that may replace silicon are being studied.

As organic materials have a large extinction coefficient and may selectively absorb light in a set or specific wavelength region according to the molecular structure thereof, organic materials may replace photodiodes and color filters concurrently (e.g., simultaneously), which may facilitate improvements in sensitivity and high integration.

An organic photodetector (OPD) including such an organic material may be applied to, for example, a display apparatus and/or an image sensor.

### SUMMARY

One or more embodiments include a compound that is depositable and absorbs light in the near-infrared region, an organic photodetector including the compound, and an electronic apparatus including the organic photodetector.

Additional aspects of embodiments will be set forth in part in the description which follows and, in part, will be apparent from the description, or may be learned by practice of the presented embodiments of the disclosure.

According to one or more embodiments,
provided is a compound represented by one selected from Formulae 1 to 4:

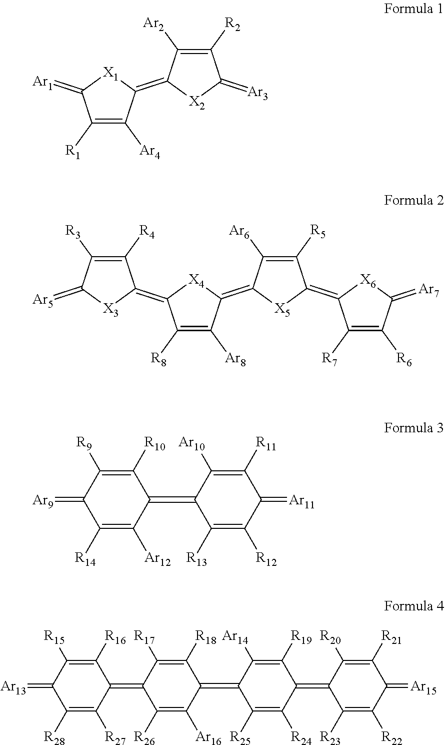

wherein, in Formulae 1 to 4,
X₁ to X₆ may each independently be selected from O, S, Se, Te, CR₃₁R₃₂, NR₃₃, BR₃₄, SiR₃₅R₃₆, and GeR₃₇R₃₈,
Ar₁, Ar₃, Ar₅, Ar₇, Ar₉, Ar₁₁, Ar₁₃, and Ar₁₅ may each independently be selected from Formulae 1-1 to 1-5,

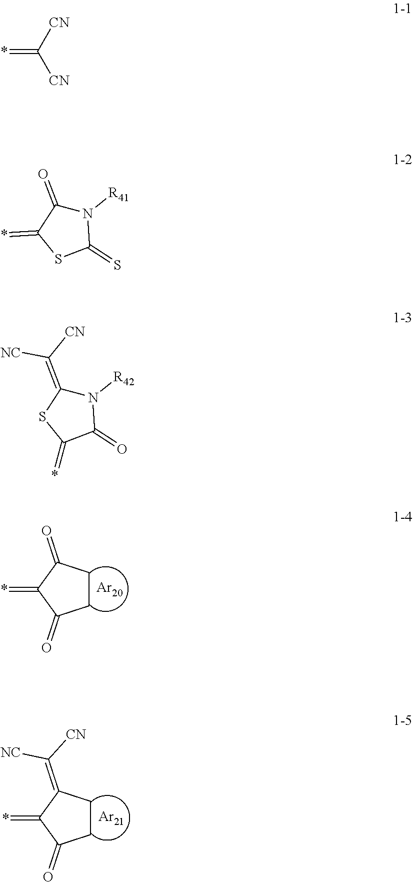

* may indicate a bond with a neighboring atom,
Ar₂, Ar₄, Ar₆, Ar₈, Ar₁₀, Ar₁₂, Ar₁₄, Ar₁₆, Ar₂₀, and Ar₂₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₁ to R₂₈, R₃₁ to R₃₈, R₄₁, and R₄₂ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
R₁₀ₐ may be:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, an organic photodetector includes:
a first electrode,
a second electrode facing the first electrode, and
an active layer between the first electrode and the second electrode,
wherein the active layer includes the compound represented by one selected from Formulae 1 to 4.

According to one or more embodiments,
an electronic apparatus includes the organic photodetector.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic cross-sectional view of a structure of an organic photodetector according to an embodiment;

FIGS. 2-3 are each a schematic cross-sectional view of a structure of an electronic apparatus according to an embodiment; and

FIGS. 4A-4B are each a view of an example of an electronic apparatus according to an embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

As the present disclosure allows for various suitable changes and numerous embodiments, example embodiments will be illustrated in the drawings and described in more detail in the written description. Effects and features of the disclosure, and methods of achieving the same will be clarified by referring to embodiments described in more detail with reference to the drawings. The subject matter of the present disclosure may, however, be embodied in many different forms and should not be construed as being limited to the embodiments set forth herein.

An expression used in the singular encompasses the expression of the plural, unless it has a clearly different meaning in the context.

It will be further understood that the terms “comprises” and/or “comprising” used herein specify the presence of stated features or elements, but do not preclude the presence or addition of one or more other features or elements.

It will be understood that when a layer, region, or element is referred to as being “on” or “onto” another layer, region, or element, it may be directly or indirectly formed on the other layer, region, or element. For example, intervening layers, regions, or elements may be present.

In descriptions with reference to the drawings, identical or corresponding elements are assigned identical or like reference numerals, and overlapping descriptions thereof may not be repeated.

Sizes of elements in the drawings may be exaggerated for convenience of explanation. In other words, because the sizes and thicknesses of elements may be arbitrarily illustrated in the drawings for convenience of explanation, and the disclosure is not limited thereto.

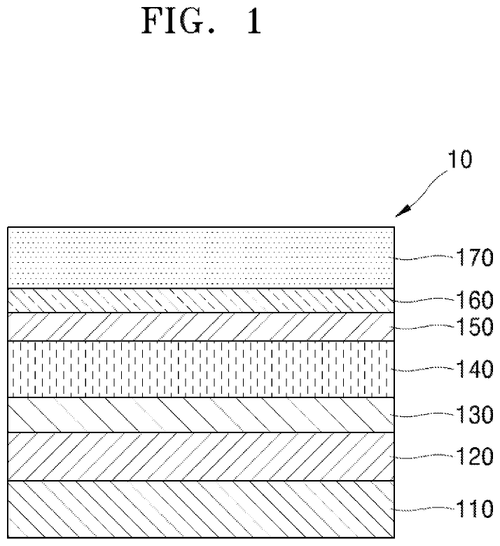

### Description of FIG. 1

FIG. 1 is a schematic cross-sectional view of an organic photodetector 10 according to an embodiment.

Referring to FIG. 1, the organic photodetector 10 according to an embodiment may include: a first electrode 110; a second electrode 170 facing the first electrode 110; an active layer 140 between the first electrode 110 and the second electrode 170; an electron injection layer 160, an electron transport layer 150 and a buffer layer, which are between the active layer 140 and the second electrode 170; and a hole injection layer 120, a hole transport layer 130, and an auxiliary layer, which are between the first electrode 110 and the active layer 140.

In FIG. 1, for example, in some cases, the electron injection layer 160, the electron transport layer 150, the buffer layer, the hole injection layer 120, the hole transport layer 130, and the auxiliary layer may each independently be present or may not each independently be present.

Recently, organic photodetectors have been formed together with organic light-emitting devices to develop sensors.

By using organic materials that absorb light of an organic light-emitting device with high luminescence efficiency, devices capable of exhibiting high absorption efficiency, for example, high external quantum efficiency (EQE), in organic photodetectors to which common layers (e.g., hole transport layers and electron transport layers) of the organic light-emitting device are applied have been developed.

A monomer for deposition requires or has a molecular weight of 1,500 g/mol or less. To absorb light in the near-infrared region, the effective conjugation length needs to be increased or may be increased to reduce the band gap. However, designing a molecule that absorbs light in a region reaching the near-infrared region by controlling the effective conjugation length within a limited molecular weight (e.g., a molecular weight of 1,500 g/mol or less) is difficult.

In an embodiment, the active layer 140 may include a compound represented by one selected from Formulae 1 to 4:

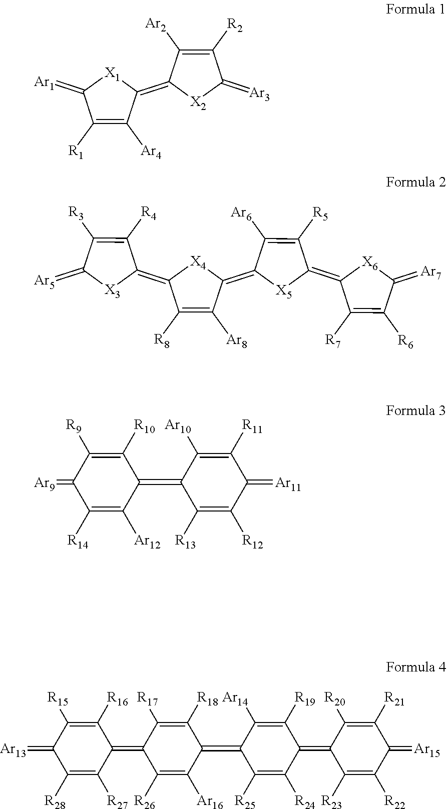

wherein, in Formulae 1 to 4,
X₁ to X₆ may each independently be selected from O, S, Se, Te, CR₃₁R₃₂, NR₃₃, BR₃₄, SiR₃₅R₃₆, and GeR₃₇R₃₈,
Ar₁, Ar₃, Ar₅, Ar₇, Ar₉, Ar₁₁, Ar₁₃, and Ar₁₅ may each independently be selected from Formulae 1-1 to 1-5,

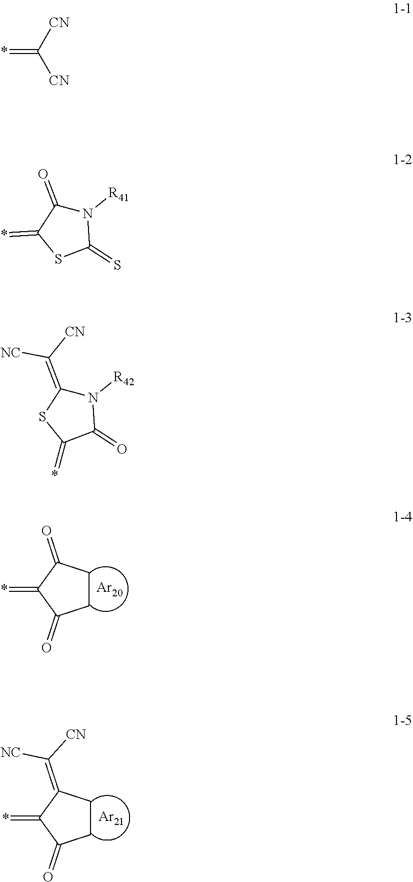

* may indicate a bond with a neighboring atom,
Ar₂, Ar₄, Ar₆, Ar₈, Ar₁₀, Ar₁₂, Ar₁₄, Ar₁₆, Ar₂₀, and Ar₂₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₁ to R₂₈, R₃₁ to R₃₈, R₄₁, and R₄₂ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁a heterocycloalkyl group unsubstituted or substituted with at least one R₁₀ₐ, a C₃-C₁a cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In Formulae 1 to 4, which have quinoid structures, a band gap tends to decrease compared to an aromatic structure, and thus, light in the near-infrared region may be absorbed even with a relatively small molecular weight. In addition, because there is no (or substantially no) absorption of light in the visible light region, and light in only the green, red, and/or near-infrared regions is absorbed, the green, red, and/or near-infrared regions may be selectively sensed. In the structures of Formulae 1 to 4, due to enhanced π-π stacking through molecular planarization, high crystallinity and charge mobility may be secured.

It is confirmed that the compound represented by one selected from Formulae 1 to 4 according to an embodiment has a longer absorption wavelength and a shorter band gap than a benzenoid compound having an aromatic structure. This feature will be further described below.

In an embodiment, Ar₂, Ar₄, Ar₆, Ar₈, Ar₁₀, Ar₁₂, Ar₁₄, Ar₁₆, Ar₂₀, and Ar₂₁ may each independently be hydrogen, deuterium, a C₆-C₆₀ aryl group unsubstituted or substituted with at least one R₁₀ₐ, or a C₁-C₆₀ heteroaryl group unsubstituted or substituted with at least one R₁₀ₐ (wherein R₁₀ₐ may be as defined herein).

For example, Ar₂, Ar₄, Ar₆, Ar₈, Ar₁₀, Ar₁₂, Ar₁₄, Ar₁₆, Ar₂₀, and Ar₂₁ may each independently be selected from hydrogen, deuterium, and Formulae 2-1 to 2-8:

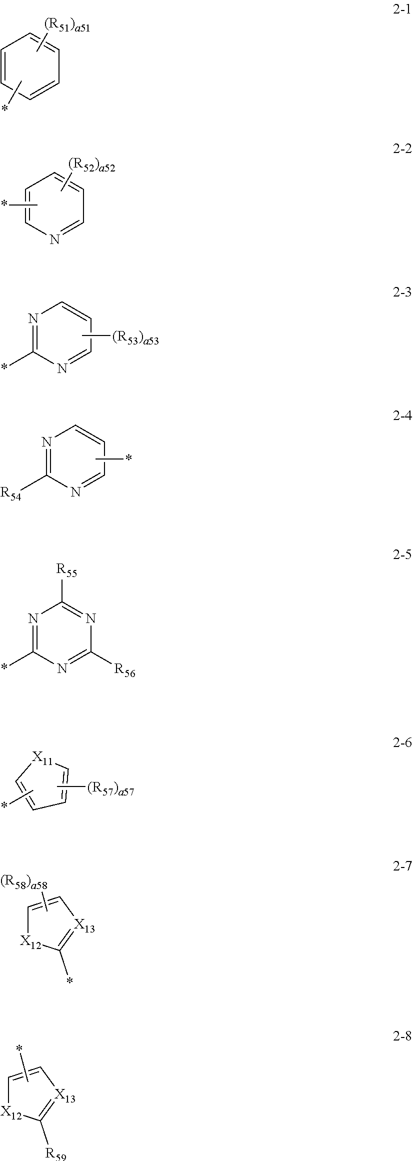

wherein, in Formulae 2-1 to 2-8, X₁₁ to X₁₃ may each independently indicate N, O, or S, and R₅₁ to R₅₉ may each independently be selected from hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
a51 may be an integer from 1 to 5,
a52 may be an integer from 1 to 4,
a53 and a57 may each independently be an integer from 1 to 3,
a58 may be 1 or 2, and
Q₁ to Q₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, R₁ to R₂₈ may each independently be hydrogen or deuterium.

In an embodiment, R₃₁ to R₃₈ may each independently be hydrogen, deuterium, —F, —Cl, —Br, —I, a C₁-C₆₀ alkyl group, a C₁-C₆₀ alkoxy group, a C₆-C₆₀ aryl group, or a C₆-C₆₀ aryloxy group.

In an embodiment, Ar₂₀ and Ar₂₁ may each independently be a C₆-C₆₀ aryl group unsubstituted or substituted with at least one R₁₀ₐ.

In an embodiment, R₄₁ and R₄₂ may each independently be a C₁-C₆₀ alkyl group unsubstituted or substituted with at least one R₁₀ₐ or a C₆-C₆₀ aryl group unsubstituted or substituted with at least one R₁₀ₐ.

In an embodiment, the compound represented by one selected from Formulae 1 to 4 may have C2 symmetry. The wording “have C2 symmetry” means that when the compound is rotated 180 degrees on the central axis of the compound, the compound is the same (e.g., appears the same) before and after the rotation. For example, in some embodiments the compound having C2 symmetry may have a two-fold rotational symmetry and may or may not have a plane of symmetry (e.g., may or may not have mirror symmetry).

In an embodiment, a molecular weight of the compound represented by one selected from Formulae 1 to 4 may be 1,500 g/mol or less. For example, the molecular weight of the compounds represented by Formulae 1 to 4 may be in a range of about 250 g/mol to about 1,500 g/mol. Because the molecular weight is 1,500 g/mol or less, there is no (or substantially no) difficulty in deposition (e.g., the compound represented by Formulae 1 to 4 is suitable for deposition).

In an embodiment, a highest occupied molecular orbital (HOMO) energy level of the compound represented by one selected from Formulae 1 to 4 may be in a range of about −6.5 eV to about −4.5 eV, and a lowest unoccupied molecular orbital (LUMO) energy level thereof may be in a range of about −4.7 eV to about −3.0 eV.

In an embodiment, an optical band gap of the compound represented by one selected from Formulae 1 to 4 may be 2.0 eV or less. Because the compound represented by one selected from Formulae 1 to 4 has a reduced optical band gap compared to a compound having an aromatic structure, the compound may absorb light to or in the near-infrared region even though it has a small molecular weight.

In an embodiment, a decomposition temperature of the compound represented by one selected from Formulae 1 to 4 may be 200° C. or higher. The compound represented by one selected from Formulae 1 to 4 has high crystallinity due to enhanced π-π stacking through molecular planarization, thereby having a high decomposition temperature (e.g., 200° C. to 350° C.), so that there is no (or substantially no) problem in a deposition process (e.g., the composition is suitable for deposition). For example, the decomposition temperature of the compound represented by one selected from Formulae 1 to 4 may be 290° C. or higher.

In an embodiment, the compound represented by one selected from Formulae 1 to 4 may be selected from the following compounds:

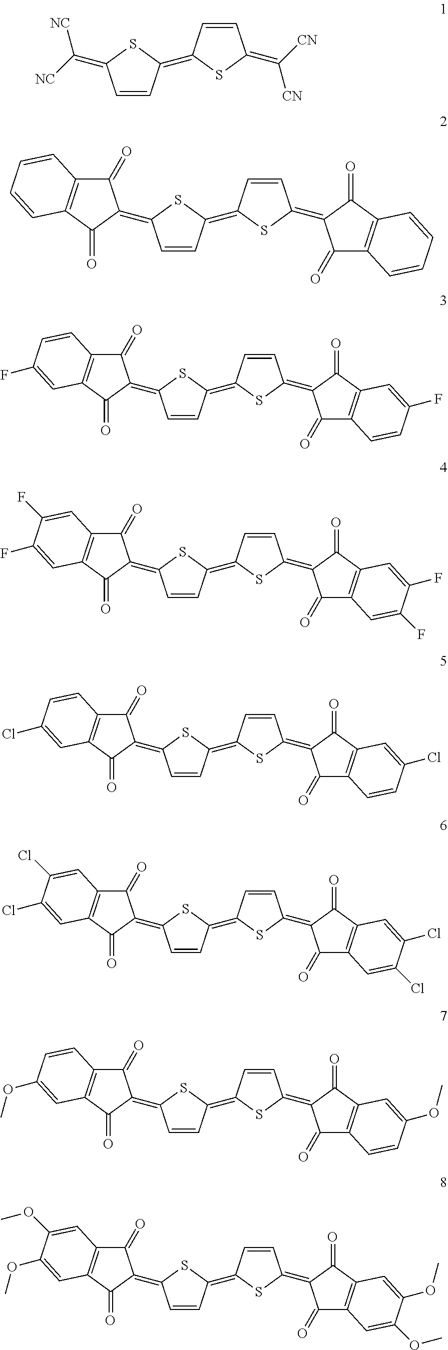

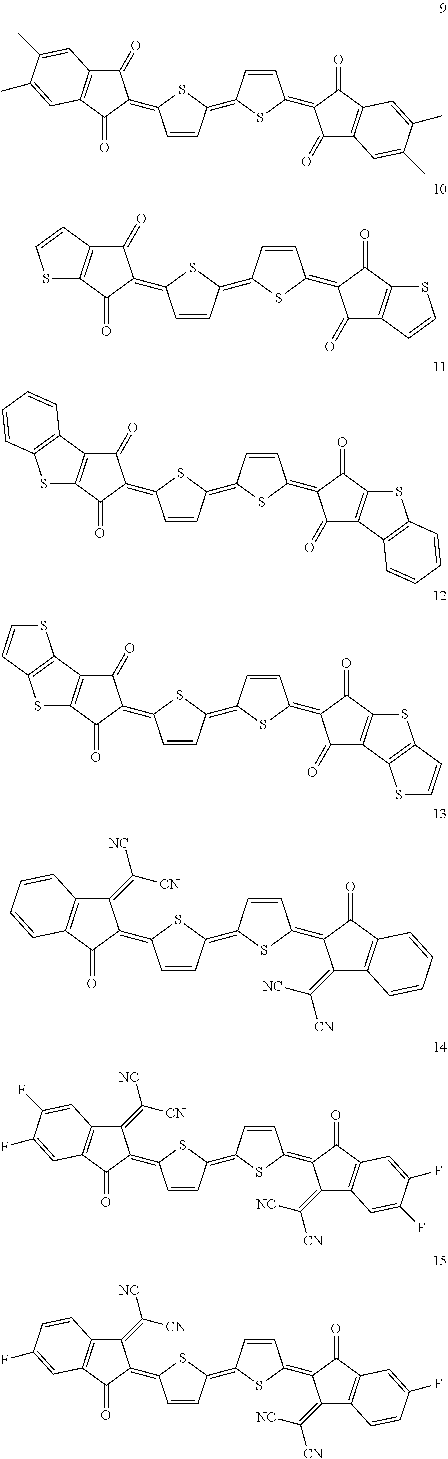

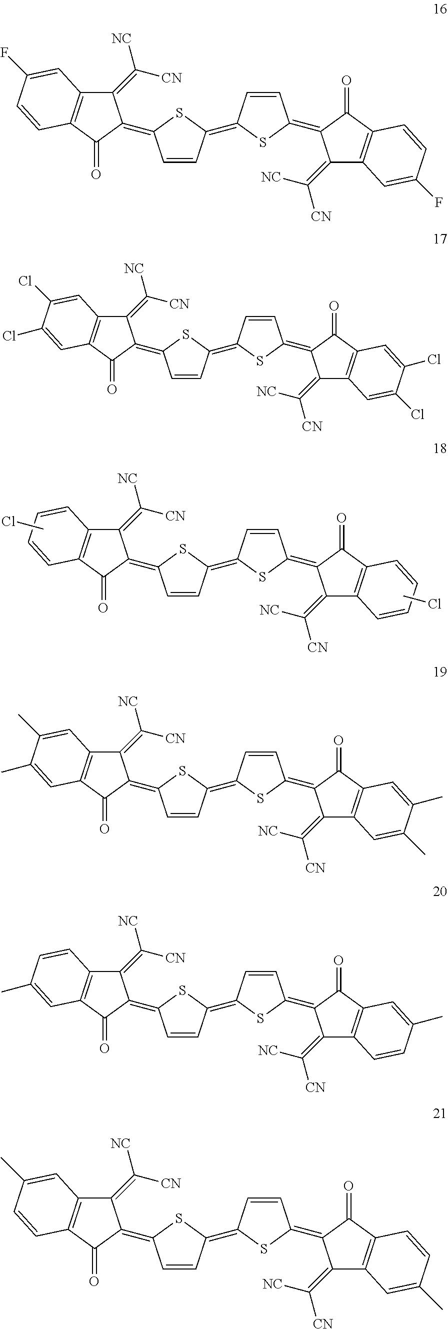

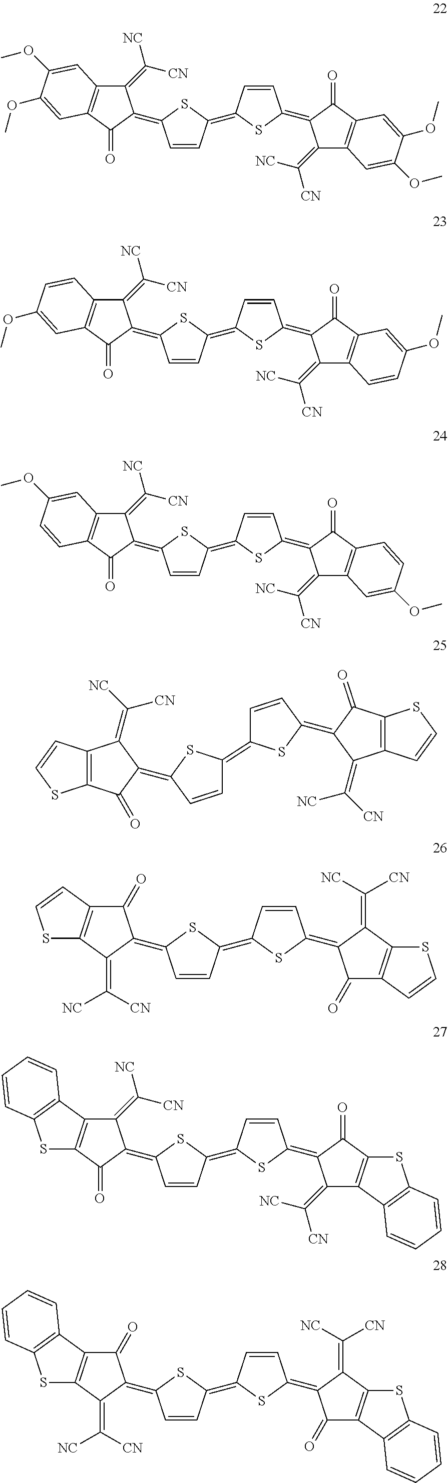

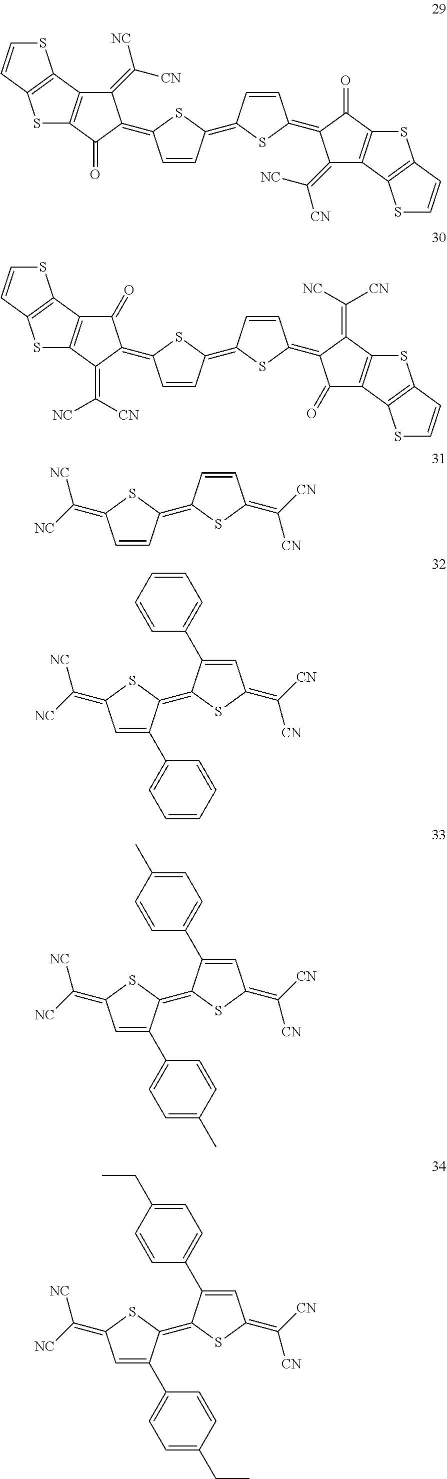

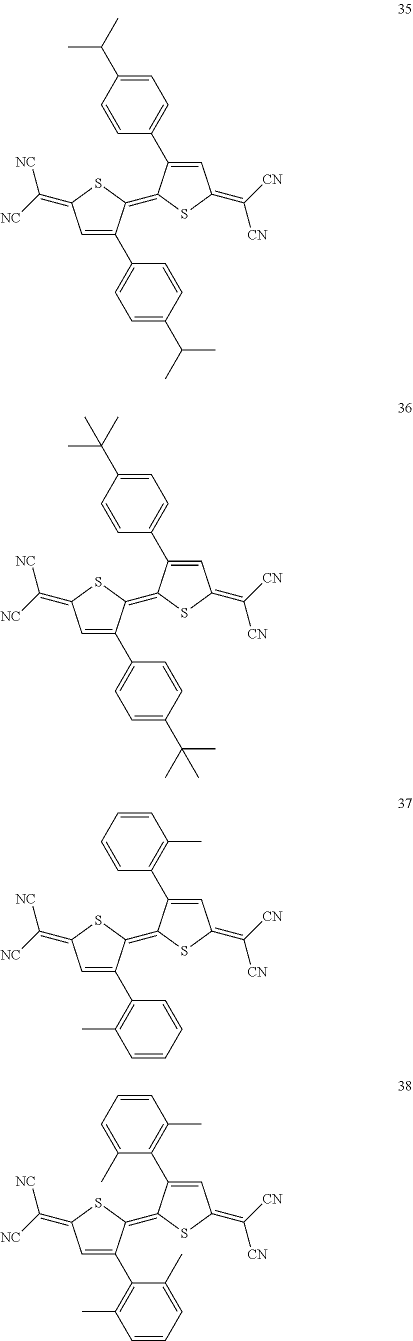

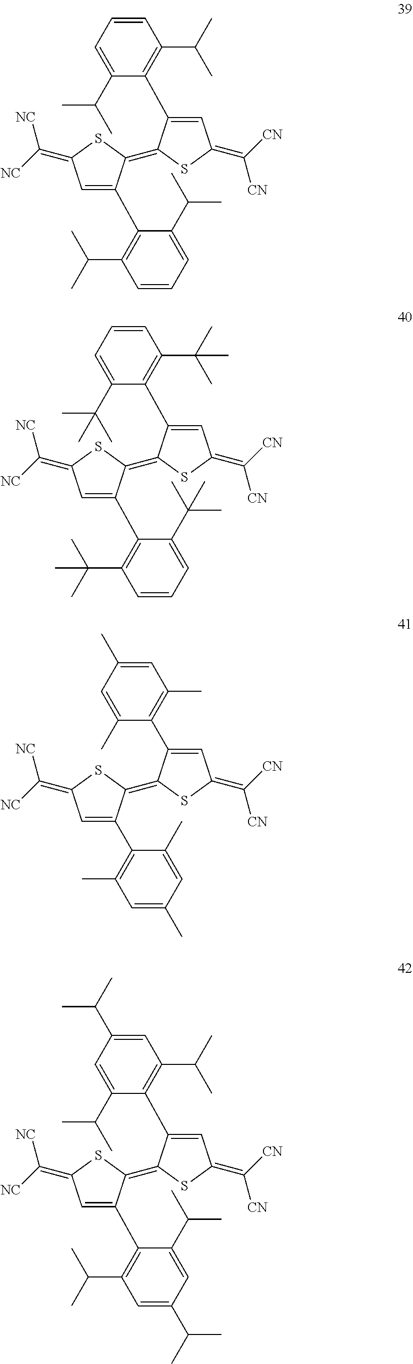

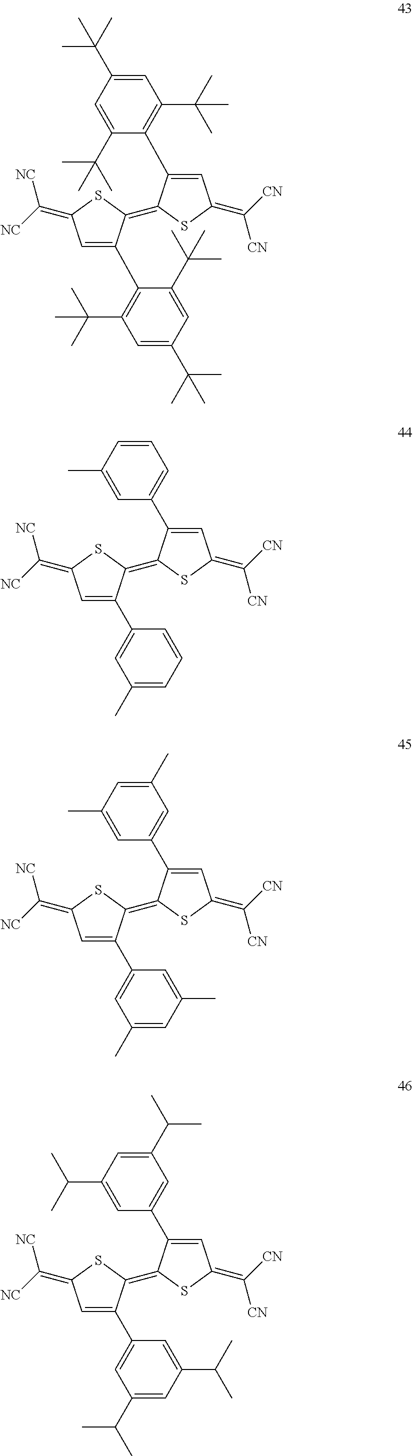

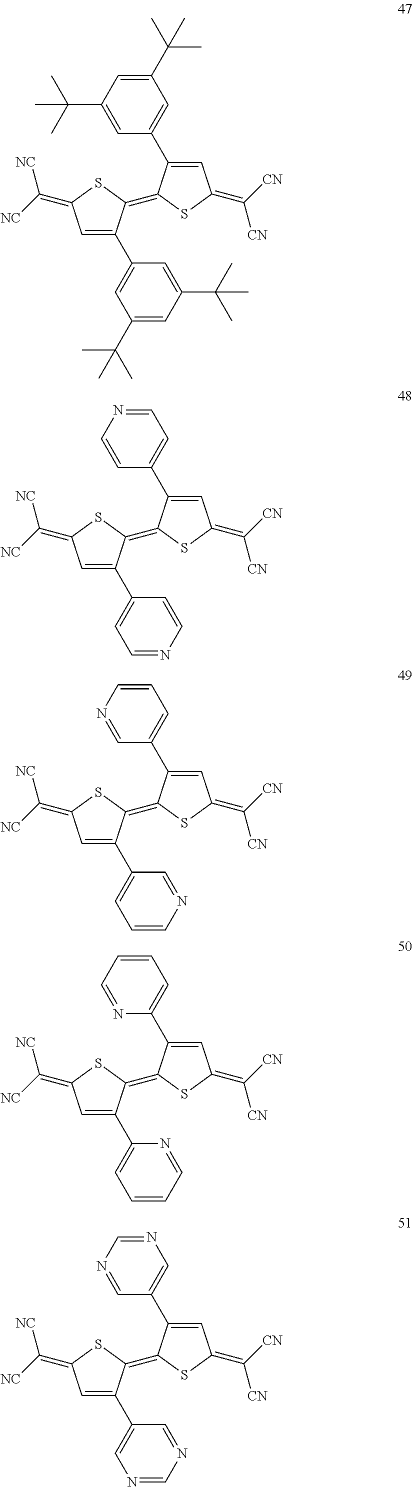

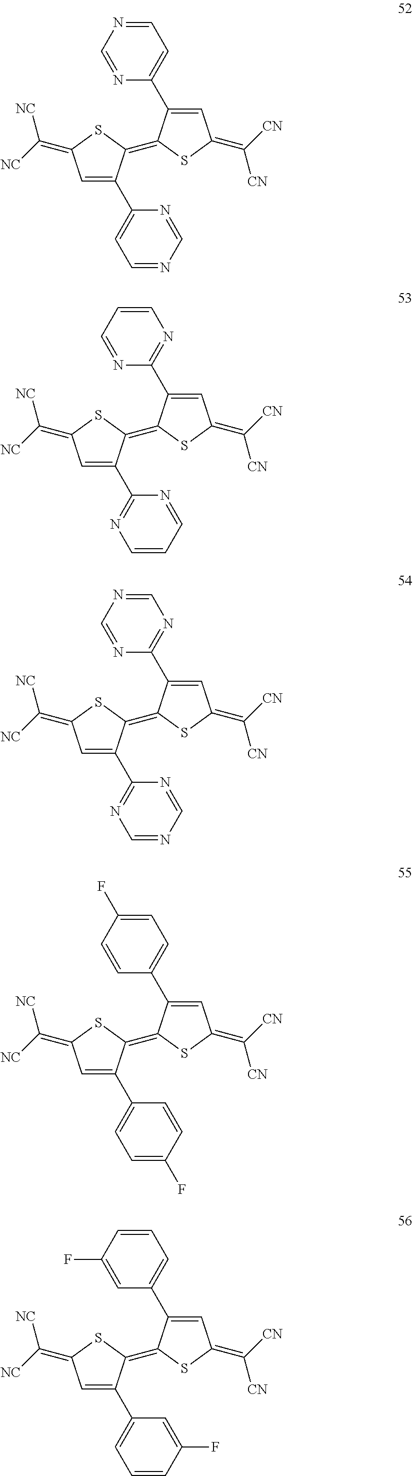

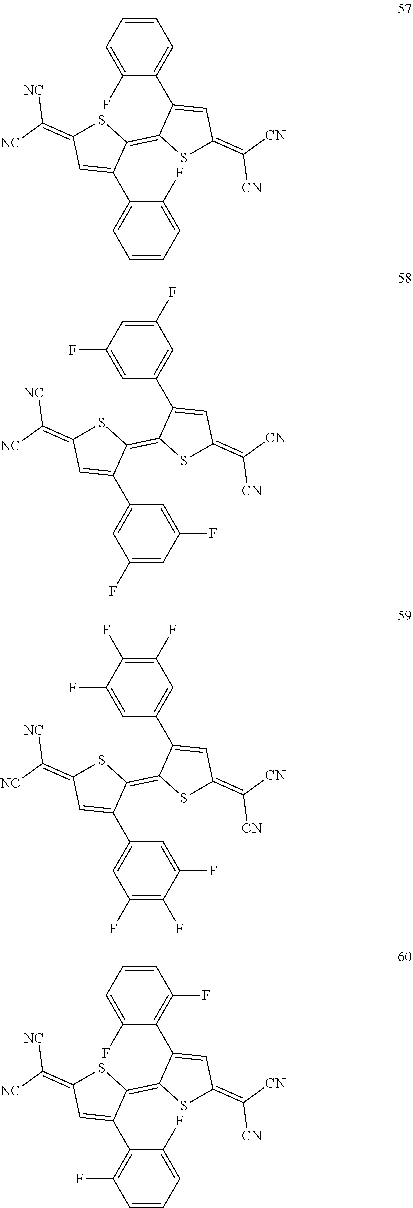

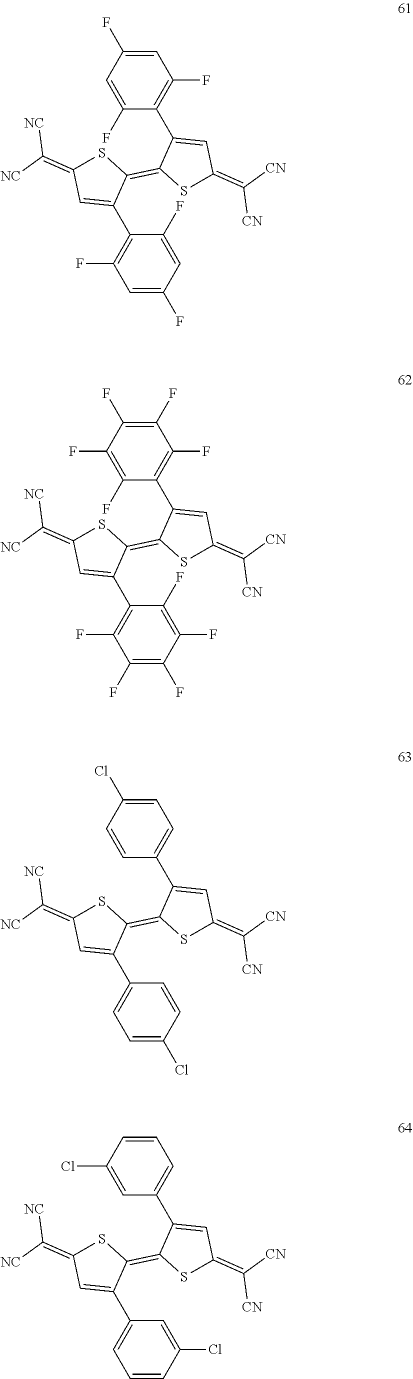 [SMILES: N#CC(C#N)=c1cc(-c2c(F)cc(F)cc2F)/c(=c2\sc(=C(C#N)C#N)cc2-c2c(F)cc(F)cc2F)s1 ; N#CC(C#N)=c1cc(-c2c(F)c(F)c(F)c(F)c2F)/c(=c2\sc(=C(C#N)C#N)cc2-c2c(F)c(F)c(F)c(F)c2F)s1 ; N#CC(C#N)=c1cc(-c2ccc(Cl)cc2)/c(=c2\sc(=C(C#N)C#N)cc2-c2ccc(Cl)cc2)s1 ; N#CC(C#N)=c1cc(-c2cccc(Cl)c2)/c(=c2\sc(=C(C#N)C#N)cc2-c2cccc(Cl)c2)s1]

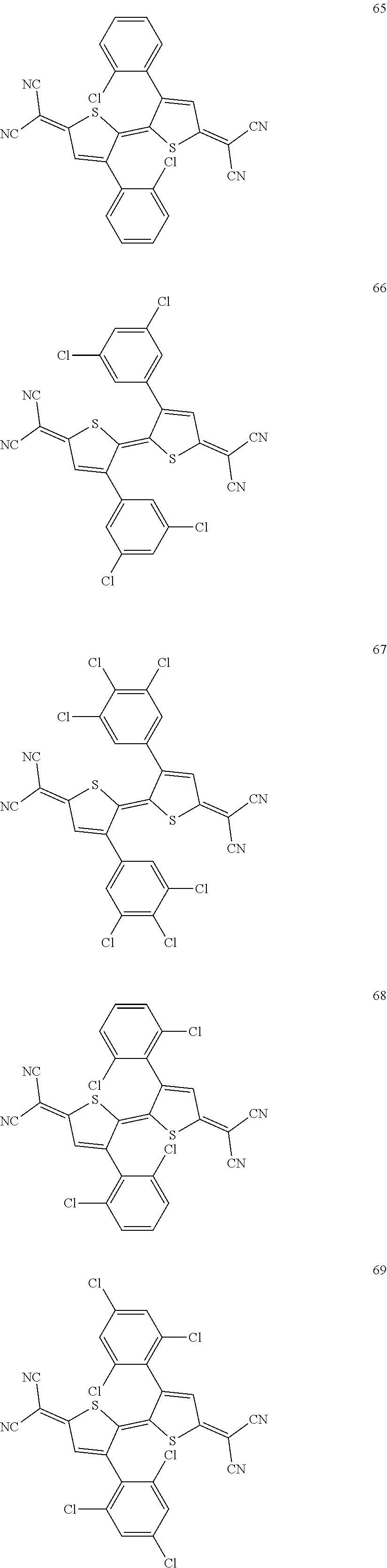 [SMILES: N#CC(C#N)=c1cc(-c2ccccc2Cl)/c(=c2\sc(=C(C#N)C#N)cc2-c2ccccc2Cl)s1 ; N#CC(C#N)=c1cc(-c2cc(Cl)cc(Cl)c2)/c(=c2\sc(=C(C#N)C#N)cc2-c2cc(Cl)cc(Cl)c2)s1 ; N#CC(C#N)=c1cc(-c2cc(Cl)c(Cl)c(Cl)c2)/c(=c2\sc(=C(C#N)C#N)cc2-c2cc(Cl)c(Cl)c(Cl)c2)s1 ; N#CC(C#N)=c1cc(-c2c(Cl)cccc2Cl)/c(=c2\sc(=C(C#N)C#N)cc2-c2c(Cl)cccc2Cl)s1 ; N#CC(C#N)=c1cc(-c2c(Cl)cc(Cl)cc2Cl)/c(=c2\sc(=C(C#N)C#N)cc2-c2c(Cl)cc(Cl)cc2Cl)s1]

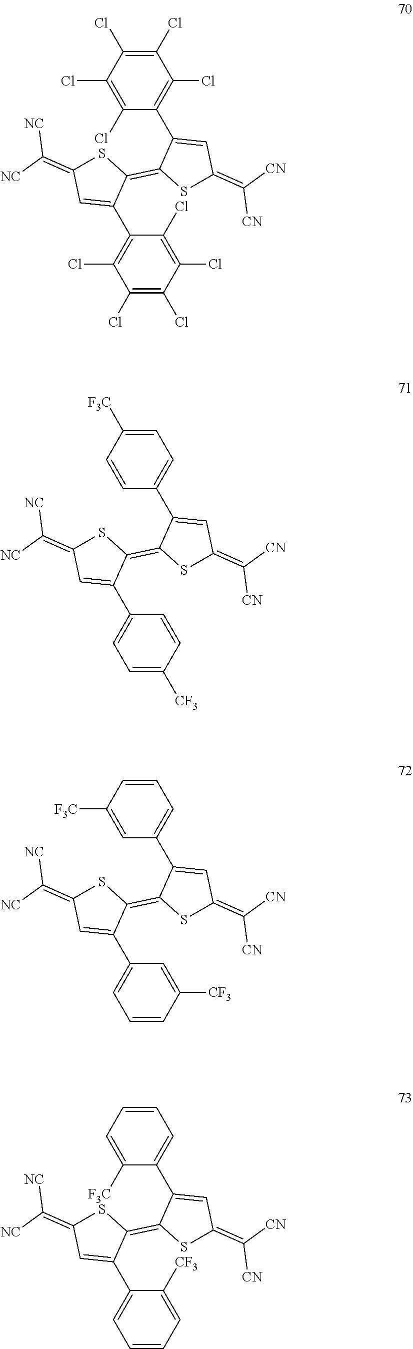 [SMILES: N#CC(C#N)=c1cc(-c2c(Cl)c(Cl)c(Cl)c(Cl)c2Cl)/c(=c2\sc(=C(C#N)C#N)cc2-c2c(Cl)c(Cl)c(Cl)c(Cl)c2Cl)s1 ; N#CC(C#N)=c1cc(-c2ccc(C(F)(F)F)cc2)/c(=c2\sc(=C(C#N)C#N)cc2-c2ccc(C(F)(F)F)cc2)s1 ; N#CC(C#N)=c1cc(-c2cccc(C(F)(F)F)c2)/c(=c2\sc(=C(C#N)C#N)cc2-c2cccc(C(F)(F)F)c2)s1 ; N#CC(C#N)=c1cc(-c2ccccc2C(F)(F)F)/c(=c2\sc(=C(C#N)C#N)cc2-c2ccccc2C(F)(F)F)s1]

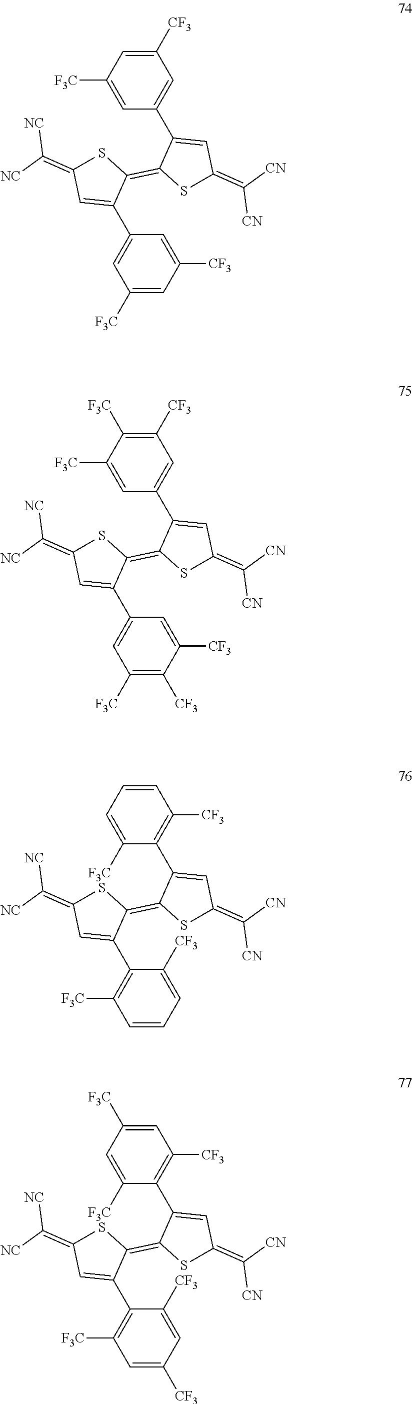 [SMILES: N#CC(C#N)=c1cc(-c2cc(C(F)(F)F)cc(C(F)(F)F)c2)/c(=c2\sc(=C(C#N)C#N)cc2-c2cc(C(F)(F)F)cc(C(F)(F)F)c2)s1 ; N#CC(C#N)=c1cc(-c2cc(C(F)(F)F)c(C(F)(F)F)c(C(F)(F)F)c2)/c(=c2\sc(=C(C#N)C#N)cc2-c2cc(C(F)(F)F)c(C(F)(F)F)c(C(F)(F)F)c2)s1 ; N#CC(C#N)=c1cc(-c2c(C(F)(F)F)cccc2C(F)(F)F)/c(=c2\sc(=C(C#N)C#N)cc2-c2c(C(F)(F)F)cccc2C(F)(F)F)s1 ; N#CC(C#N)=c1cc(-c2c(C(F)(F)F)cc(C(F)(F)F)cc2C(F)(F)F)/c(=c2\sc(=C(C#N)C#N)cc2-c2c(C(F)(F)F)cc(C(F)(F)F)cc2C(F)(F)F)s1]

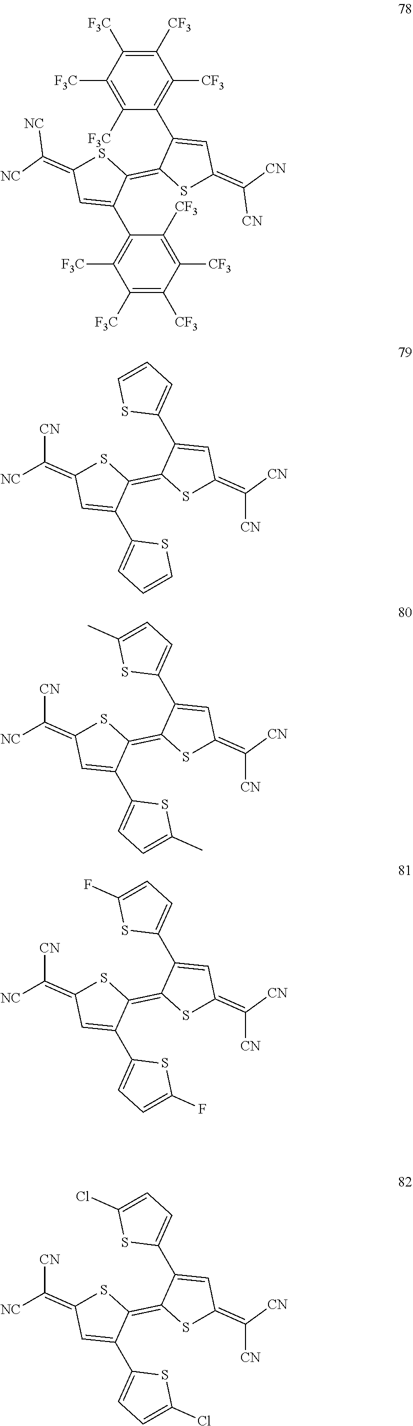

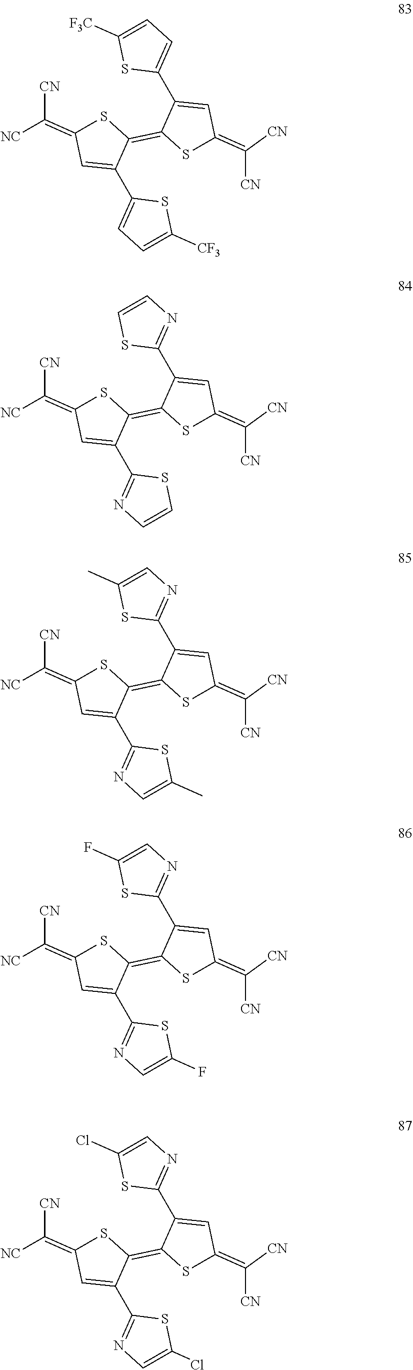

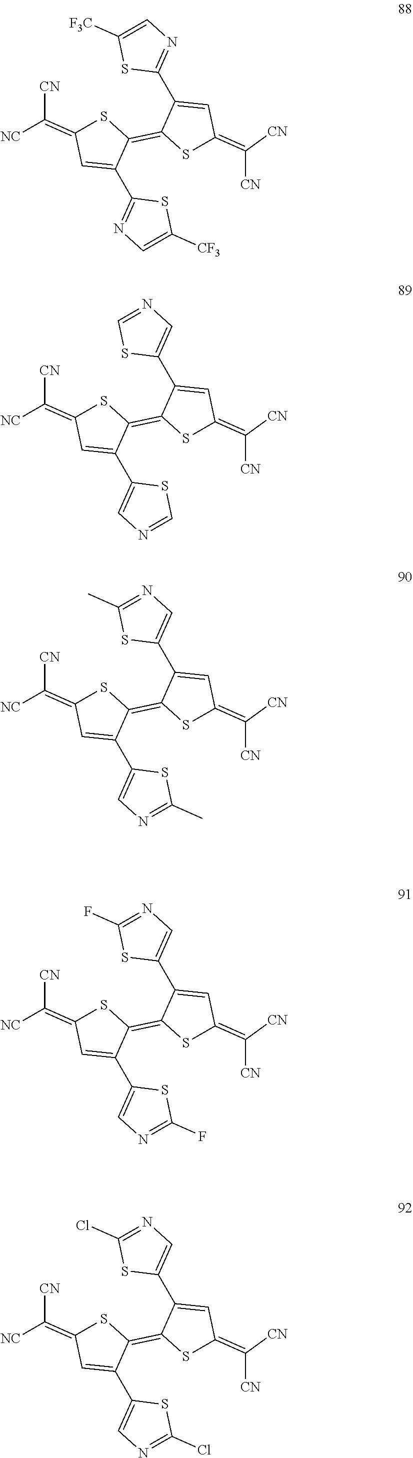 [SMILES: N#CC(C#N)=c1cc(-c2ncc(C(F)(F)F)s2)/c(=c2\sc(=C(C#N)C#N)cc2-c2ncc(C(F)(F)F)s2)s1 ; N#CC(C#N)=c1cc(-c2cncs2)/c(=c2\sc(=C(C#N)C#N)cc2-c2cncs2)s1 ; Cc1ncc(-c2cc(=C(C#N)C#N)s/c2=c2/sc(=C(C#N)C#N)cc2-c2cnc(C)s2)s1 ; N#CC(C#N)=c1cc(-c2cnc(F)s2)/c(=c2\sc(=C(C#N)C#N)cc2-c2cnc(F)s2)s1 ; N#CC(C#N)=c1cc(-c2cnc(Cl)s2)/c(=c2\sc(=C(C#N)C#N)cc2-c2cnc(Cl)s2)s1]

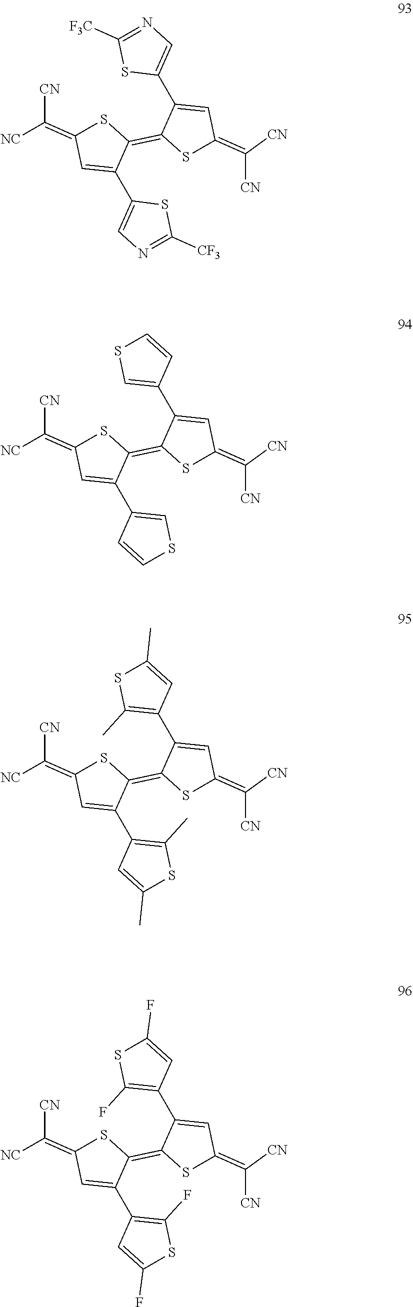 [SMILES: N#CC(C#N)=c1cc(-c2cnc(C(F)(F)F)s2)/c(=c2\sc(=C(C#N)C#N)cc2-c2cnc(C(F)(F)F)s2)s1 ; N#CC(C#N)=c1cc(-c2ccsc2)/c(=c2\sc(=C(C#N)C#N)cc2-c2ccsc2)s1 ; Cc1cc(-c2cc(=C(C#N)C#N)s/c2=c2/sc(=C(C#N)C#N)cc2-c2cc(C)sc2C)c(C)s1 ; N#CC(C#N)=c1cc(-c2cc(F)sc2F)/c(=c2\sc(=C(C#N)C#N)cc2-c2cc(F)sc2F)s1]

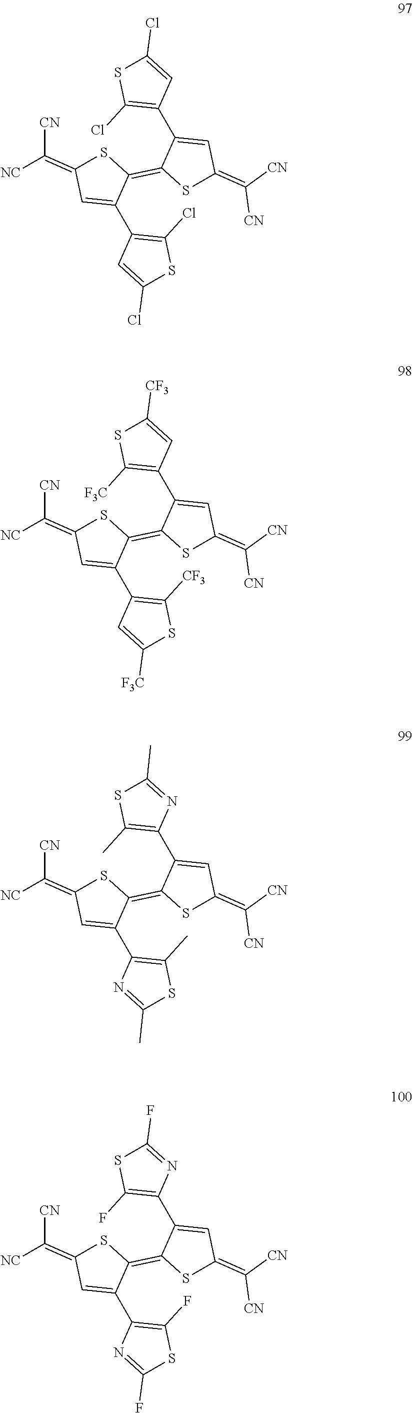 [SMILES: N#CC(C#N)=c1cc(-c2cc(Cl)sc2Cl)/c(=c2\sc(=C(C#N)C#N)cc2-c2cc(Cl)sc2Cl)s1 ; N#CC(C#N)=c1cc(-c2cc(C(F)(F)F)sc2C(F)(F)F)/c(=c2\sc(=C(C#N)C#N)cc2-c2cc(C(F)(F)F)sc2C(F)(F)F)s1 ; Cc1nc(-c2cc(=C(C#N)C#N)s/c2=c2/sc(=C(C#N)C#N)cc2-c2nc(C)sc2C)c(C)s1 ; N#CC(C#N)=c1cc(-c2nc(F)sc2F)/c(=c2\sc(=C(C#N)C#N)cc2-c2nc(F)sc2F)s1]

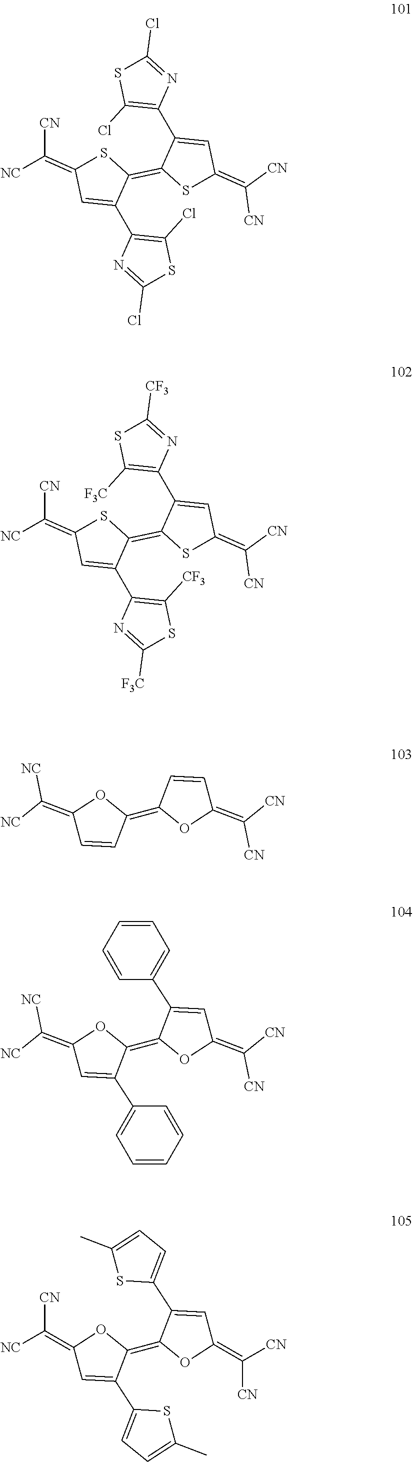 [SMILES: N#CC(C#N)=c1cc(-c2nc(Cl)sc2Cl)/c(=c2\sc(=C(C#N)C#N)cc2-c2nc(Cl)sc2Cl)s1 ; N#CC(C#N)=c1cc(-c2nc(C(F)(F)F)sc2C(F)(F)F)/c(=c2\sc(=C(C#N)C#N)cc2-c2nc(C(F)(F)F)sc2C(F)(F)F)s1 ; N#CC(C#N)=c1cc/c(=c2/ccc(=C(C#N)C#N)o2)o1 ; N#CC(C#N)=c1cc(-c2ccccc2)/c(=c2\oc(=C(C#N)C#N)cc2-c2ccccc2)o1 ; Cc1ccc(-c2cc(=C(C#N)C#N)o/c2=c2/oc(=C(C#N)C#N)cc2-c2ccc(C)s2)s1]

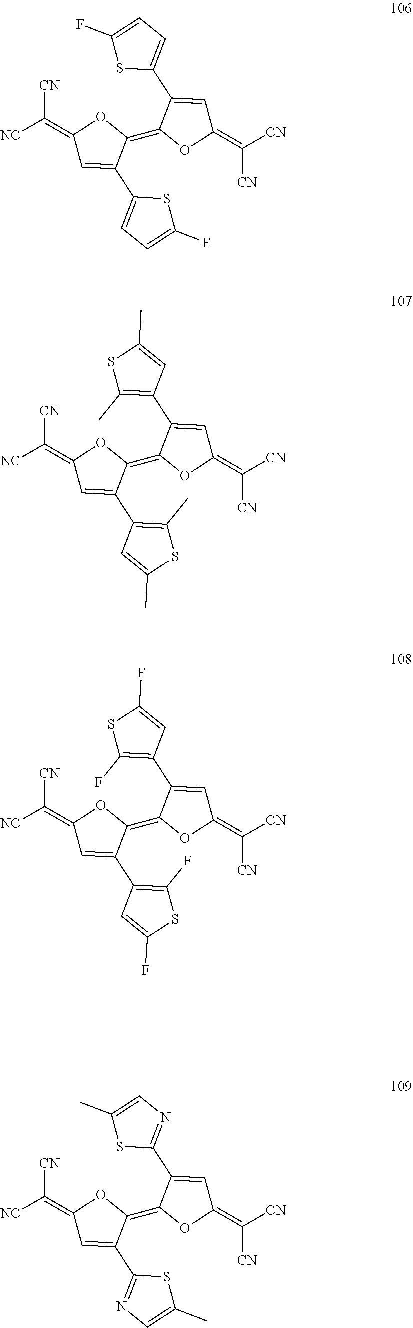 [SMILES: N#CC(C#N)=c1cc(-c2ccc(F)s2)/c(=c2\oc(=C(C#N)C#N)cc2-c2ccc(F)s2)o1 ; Cc1cc(-c2cc(=C(C#N)C#N)o/c2=c2/oc(=C(C#N)C#N)cc2-c2cc(C)sc2C)c(C)s1 ; N#CC(C#N)=c1cc(-c2cc(F)sc2F)/c(=c2\oc(=C(C#N)C#N)cc2-c2cc(F)sc2F)o1 ; Cc1cnc(-c2cc(=C(C#N)C#N)o/c2=c2/oc(=C(C#N)C#N)cc2-c2ncc(C)s2)s1]

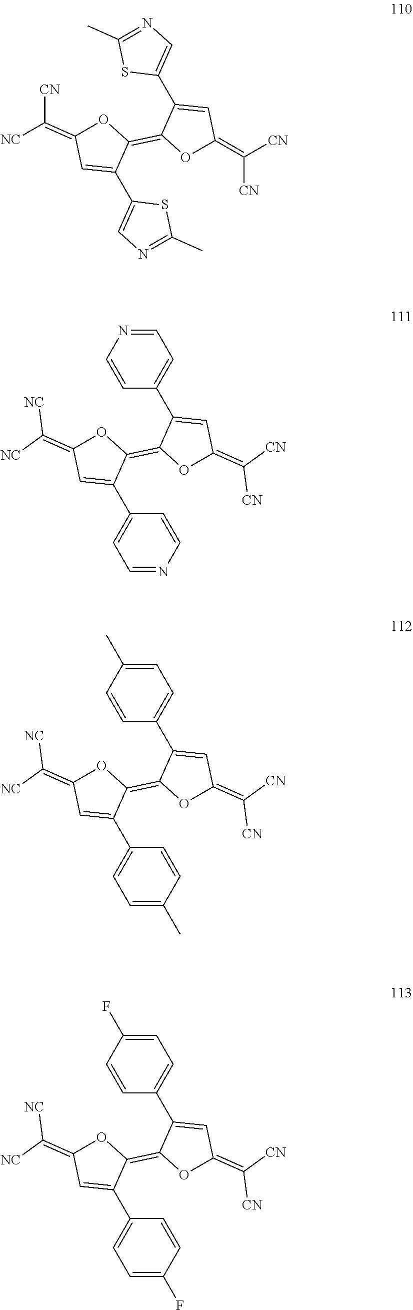 [SMILES: Cc1ncc(-c2cc(=C(C#N)C#N)o/c2=c2/oc(=C(C#N)C#N)cc2-c2cnc(C)s2)s1 ; N#CC(C#N)=c1cc(-c2ccncc2)/c(=c2\oc(=C(C#N)C#N)cc2-c2ccncc2)o1 ; Cc1ccc(-c2cc(=C(C#N)C#N)o/c2=c2/oc(=C(C#N)C#N)cc2-c2ccc(C)cc2)cc1 ; N#CC(C#N)=c1cc(-c2ccc(F)cc2)/c(=c2\oc(=C(C#N)C#N)cc2-c2ccc(F)cc2)o1]

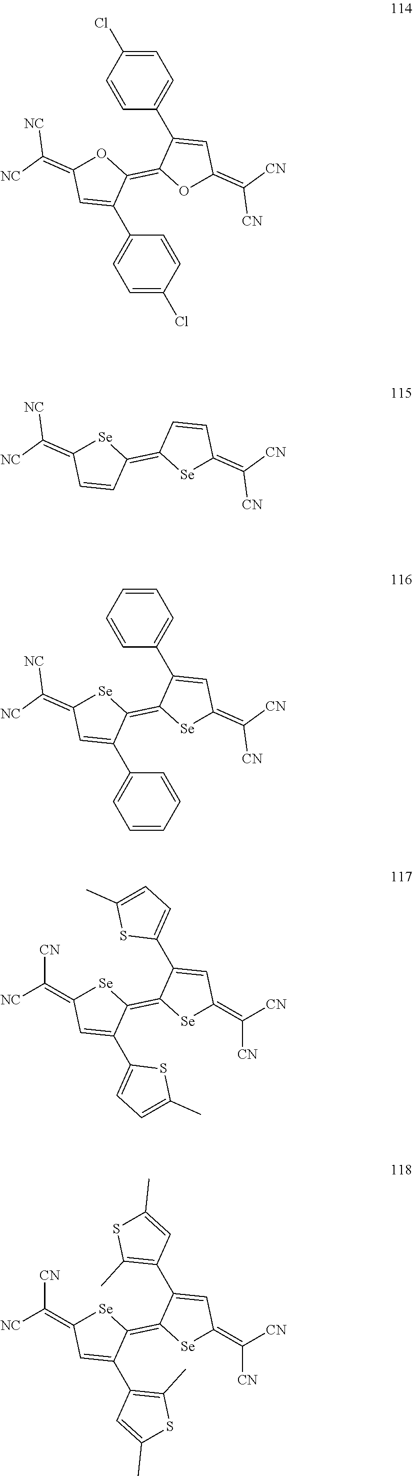 [SMILES: N#CC(C#N)=c1cc(-c2ccc(Cl)cc2)/c(=c2\oc(=C(C#N)C#N)cc2-c2ccc(Cl)cc2)o1 ; N#CC(C#N)=c1cc/c(=c2/ccc(=C(C#N)C#N)[se]2)[se]1 ; N#CC(C#N)=c1cc(-c2ccccc2)/c(=c2\[se]c(=C(C#N)C#N)cc2-c2ccccc2)[se]1 ; Cc1ccc(-c2cc(=C(C#N)C#N)[se]/c2=c2/[se]c(=C(C#N)C#N)cc2-c2ccc(C)s2)s1 ; Cc1cc(-c2cc(=C(C#N)C#N)[se]/c2=c2/[se]c(=C(C#N)C#N)cc2-c2cc(C)sc2C)c(C)s1]

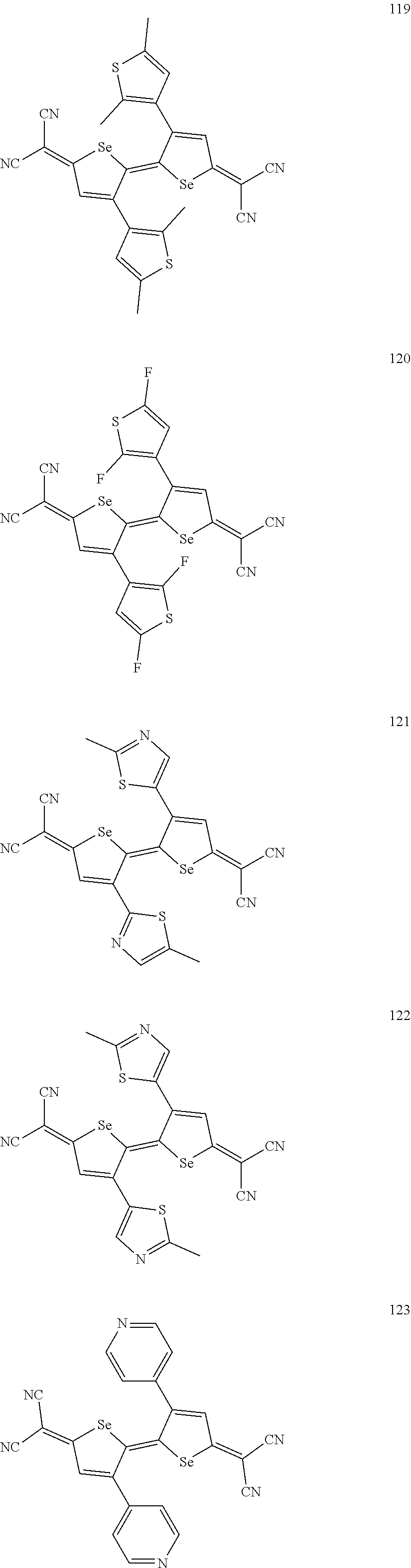 [SMILES: Cc1cc(-c2cc(=C(C#N)C#N)[se]/c2=c2/[se]c(=C(C#N)C#N)cc2-c2cc(C)sc2C)c(C)s1 ; N#CC(C#N)=c1cc(-c2cc(F)sc2F)/c(=c2\[se]c(=C(C#N)C#N)cc2-c2cc(F)sc2F)[se]1 ; Cc1cnc(-c2cc(=C(C#N)C#N)[se]/c2=c2/[se]c(=C(C#N)C#N)cc2-c2cnc(C)s2)s1 ; Cc1ncc(-c2cc(=C(C#N)C#N)[se]/c2=c2/[se]c(=C(C#N)C#N)cc2-c2cnc(C)s2)s1 ; N#CC(C#N)=c1cc(-c2ccncc2)/c(=c2\[se]c(=C(C#N)C#N)cc2-c2ccncc2)[se]1]

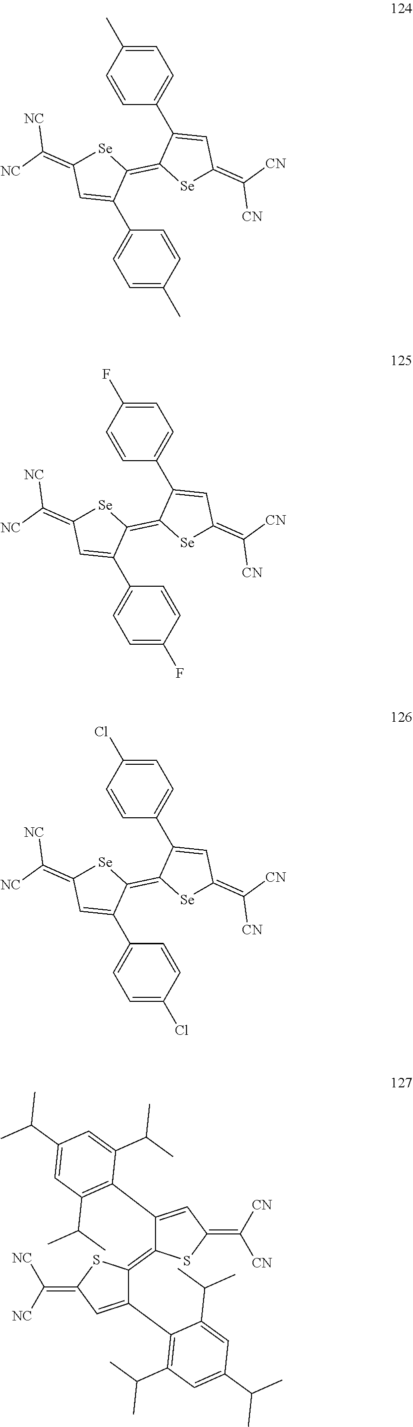 [SMILES: Cc1ccc(-c2cc(=C(C#N)C#N)[se]/c2=c2/[se]c(=C(C#N)C#N)cc2-c2ccc(C)cc2)cc1 ; N#CC(C#N)=c1cc(-c2ccc(F)cc2)/c(=c2\[se]c(=C(C#N)C#N)cc2-c2ccc(F)cc2)[se]1 ; N#CC(C#N)=c1cc(-c2ccc(Cl)cc2)/c(=c2\[se]c(=C(C#N)C#N)cc2-c2ccc(Cl)cc2)[se]1 ; CC(C)c1cc(C(C)C)c(-c2cc(=C(C#N)C#N)s/c2=c2/sc(=C(C#N)C#N)cc2-c2c(C(C)C)cc(C(C)C)cc2C(C)C)c(C(C)C)c1]

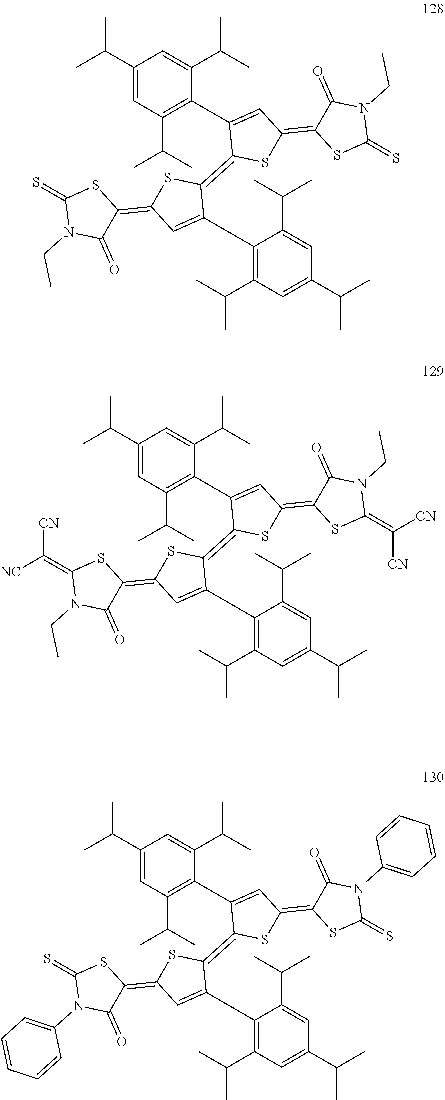 [SMILES: CCN1C(=O)/C(=c2\cc(-c3c(C(C)C)cc(C(C)C)cc3C(C)C)/c(=c3\s/c(=C4\SC(=S)N(CC)C4=O)cc3-c3c(C(C)C)cc(C(C)C)cc3C(C)C)s2)SC1=S ; CCn1c(=C(C#N)C#N)s/c(=c2/cc(-c3c(C(C)C)cc(C(C)C)cc3C(C)C)/c(=c3\s/c(=c4\sc(=C(C#N)C#N)n(CC)c4=O)cc3-c3c(C(C)C)cc(C(C)C)cc3C(C)C)s2)c1=O ; CC(C)c1cc(C(C)C)c(-c2c/c(=C3/SC(=S)N(c4ccccc4)C3=O)s/c2=c2/s/c(=C3\SC(=S)N(c4ccccc4)C3=O)cc2-c2c(C(C)C)cc(C(C)C)cc2C(C)C)c(C(C)C)c1]

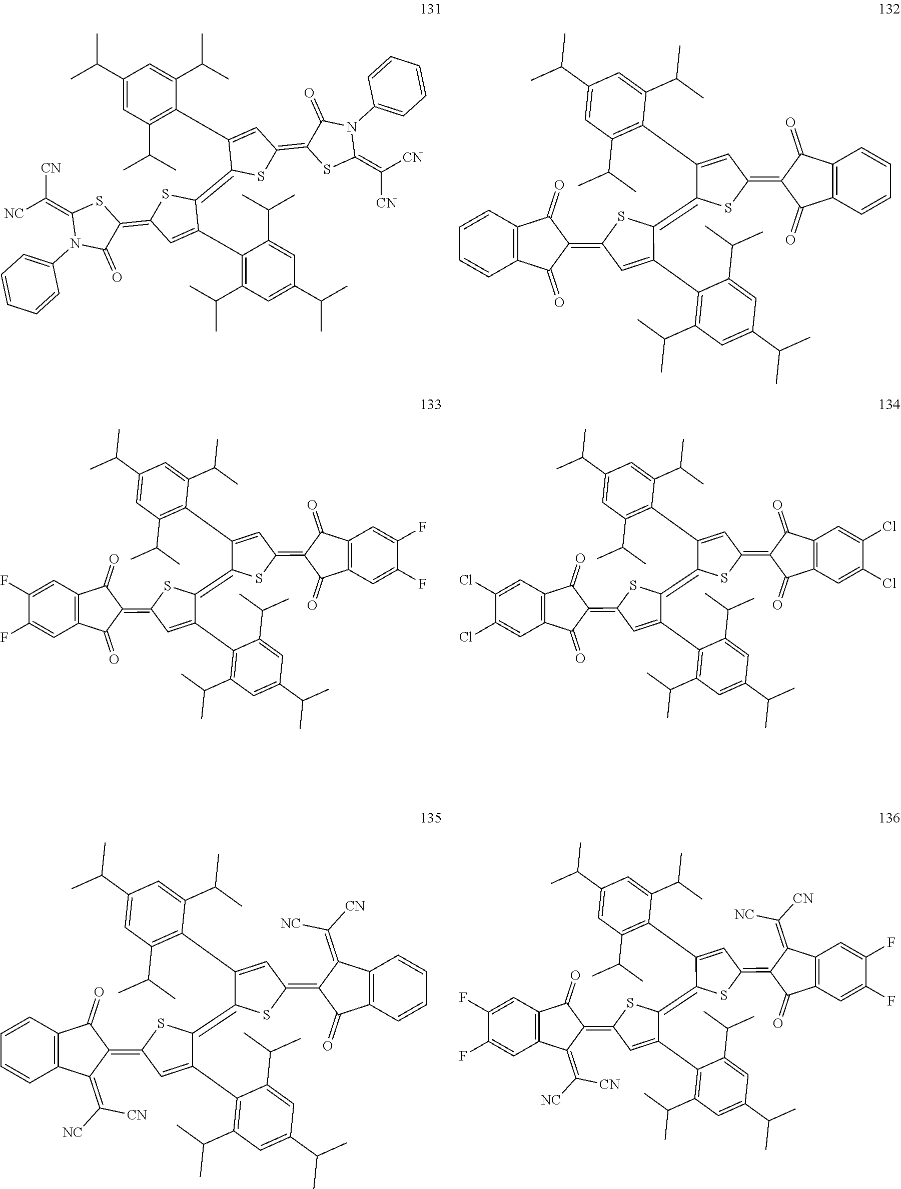

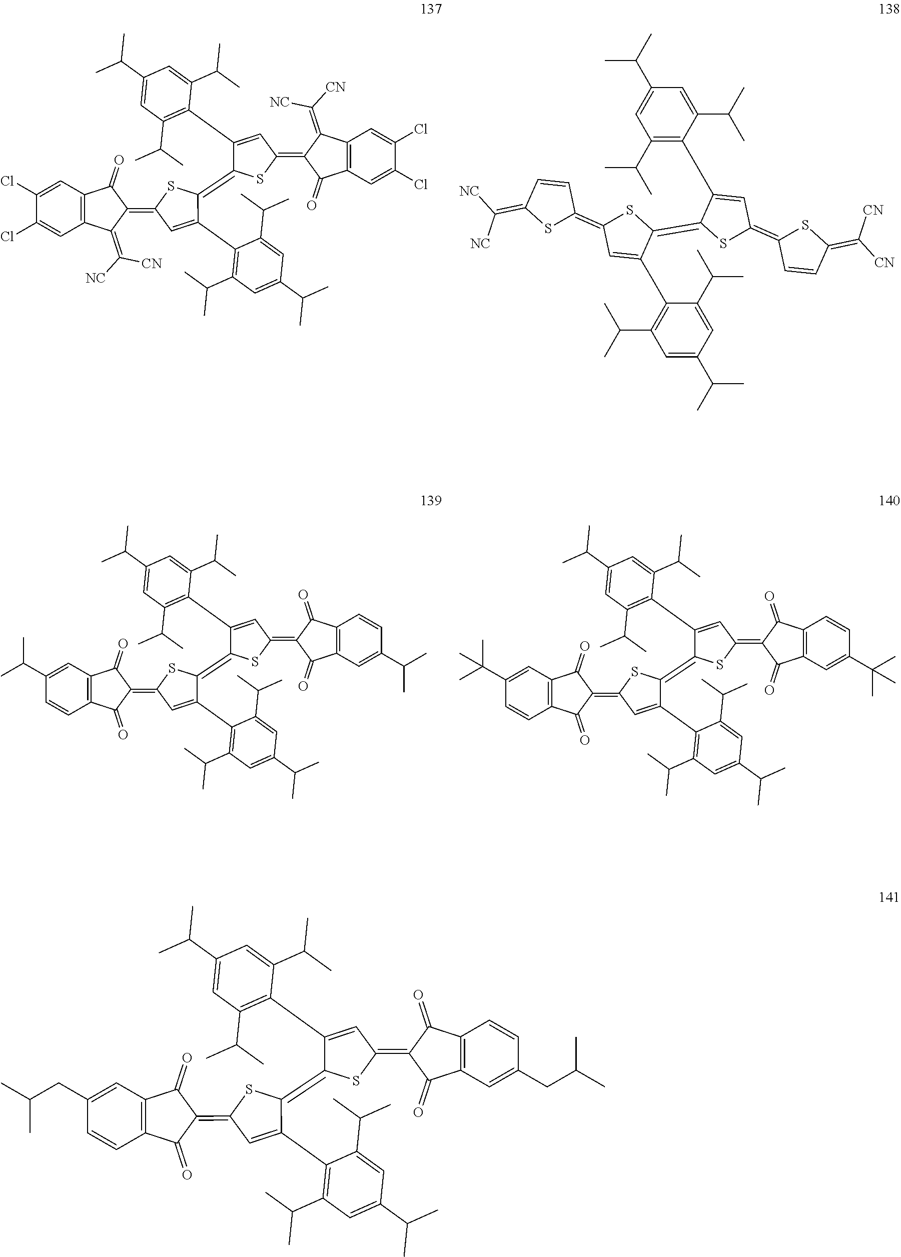

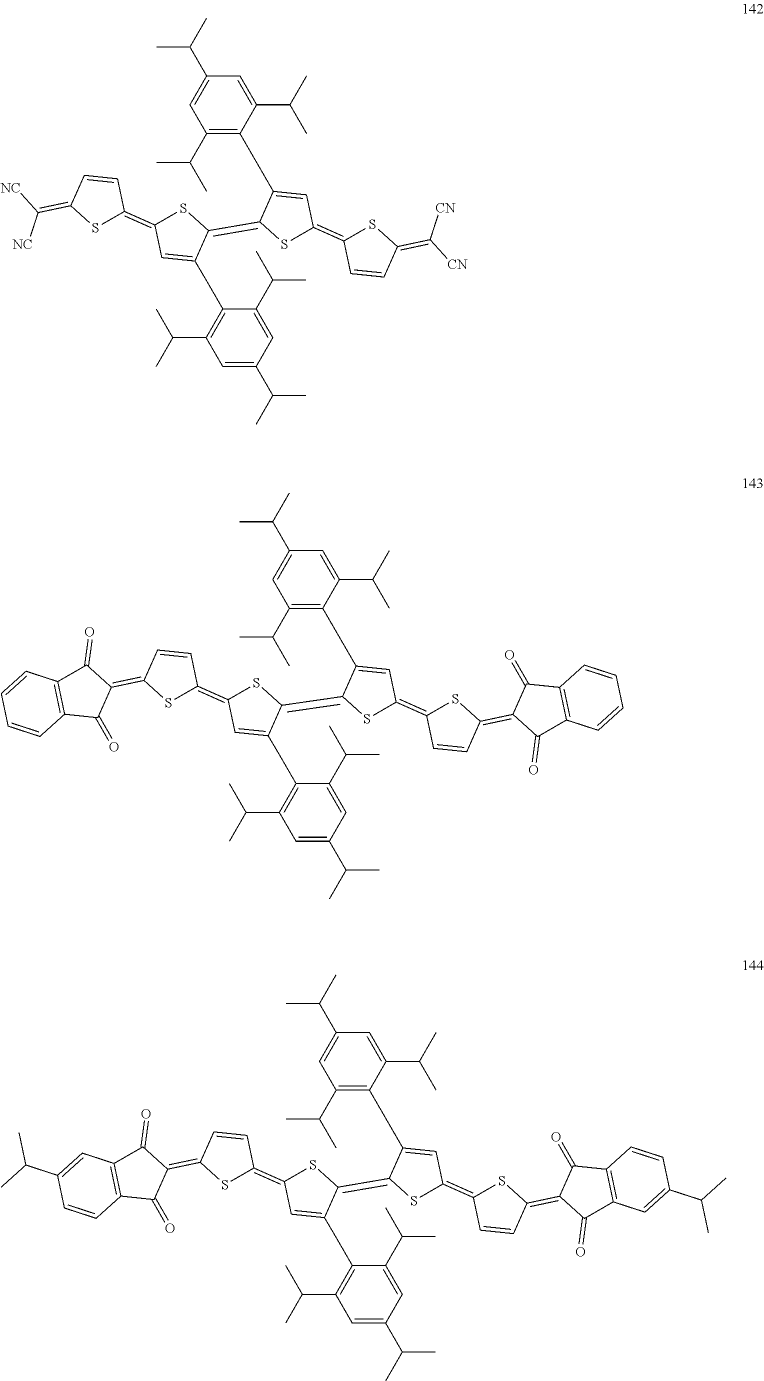 [SMILES: CC(C)c1cc(C(C)C)c(Cc2c/c(=c3/ccc(=C(C#N)C#N)s3)s/c2=c2/s/c(=c3\ccc(=C(C#N)C#N)s3)cc2Cc2c(C(C)C)cc(C(C)C)cc2C(C)C)c(C(C)C)c1 ; CC(C)c1cc(C(C)C)c(Cc2c/c(=c3/ccc(=C4C(=O)c5ccccc5C4=O)s3)s/c2=c2/s/c(=c3\ccc(=C4C(=O)c5ccccc5C4=O)s3)cc2Cc2c(C(C)C)cc(C(C)C)cc2C(C)C)c(C(C)C)c1 ; CC(C)c1ccc2c(c1)C(=O)/C(=c1\cc/c(=c3/cc(Cc4c(C(C)C)cc(C(C)C)cc4C(C)C)/c(=c4\s/c(=c5\cc/c(=C6/C(=O)c7ccc(C(C)C)cc7C6=O)s5)cc4Cc4c(C(C)C)cc(C(C)C)cc4C(C)C)s3)s1)C2=O]

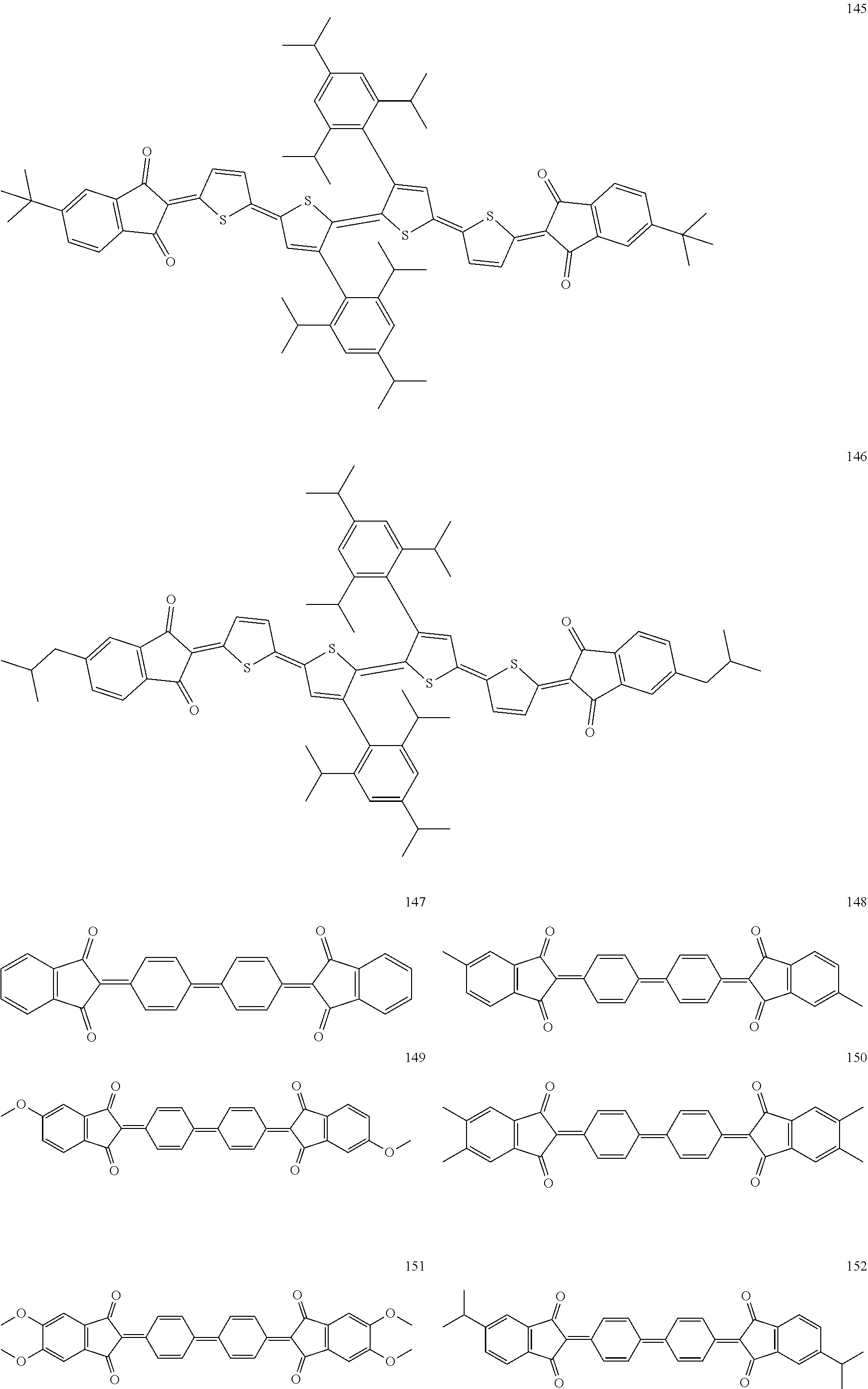

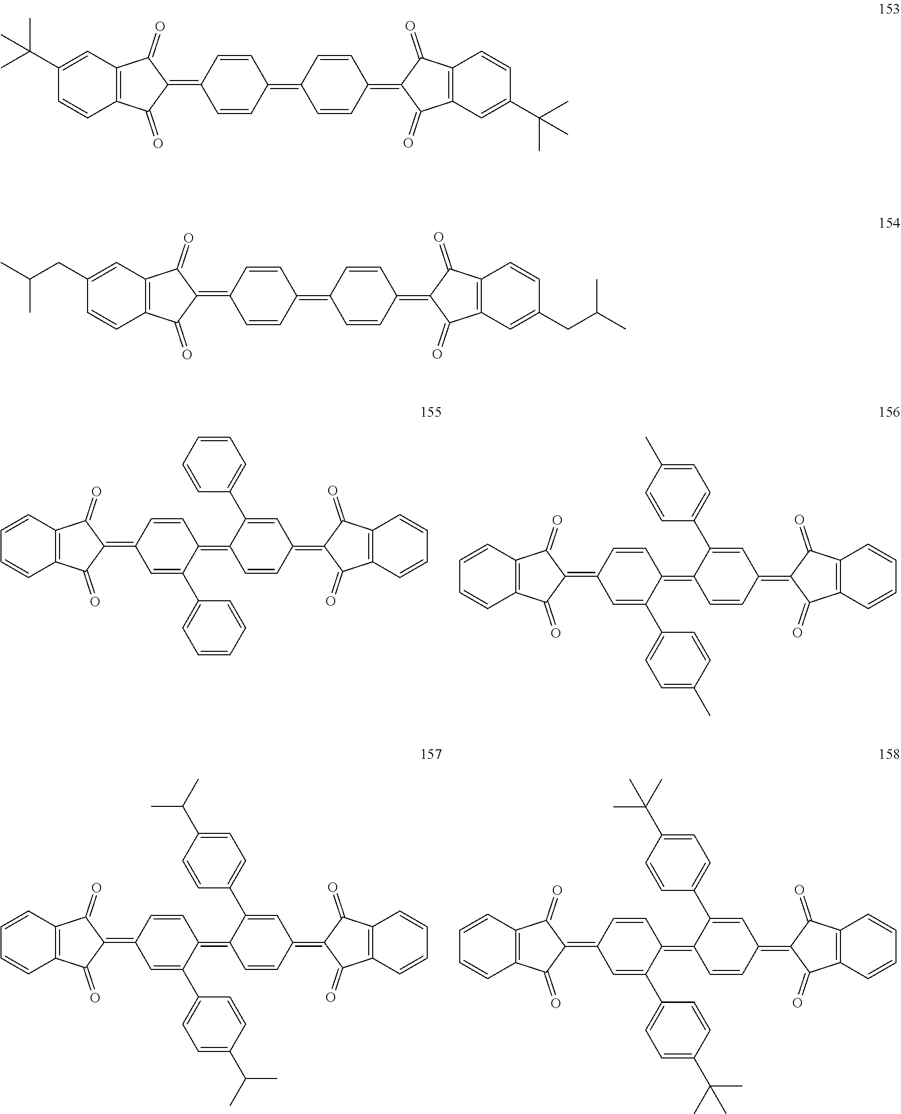

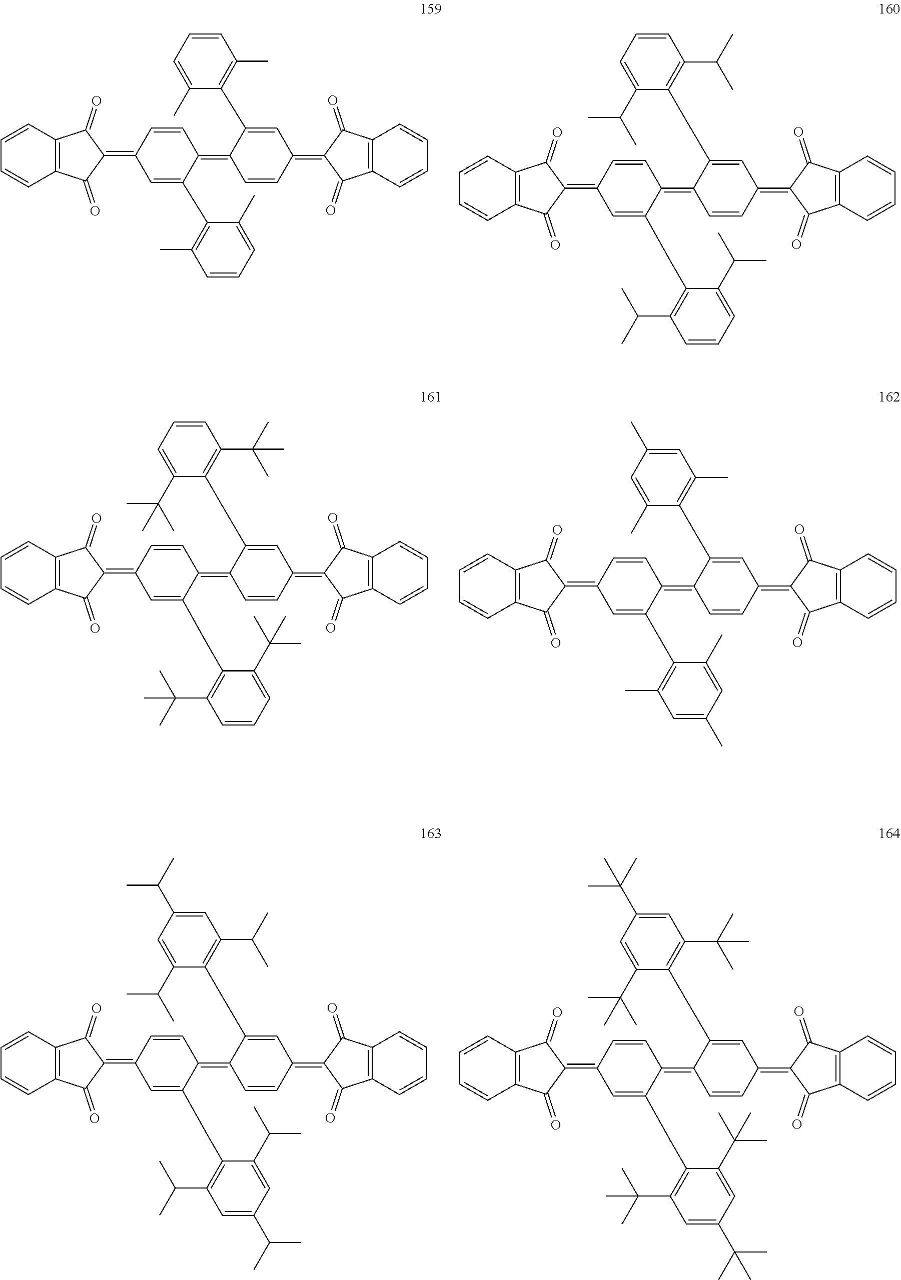

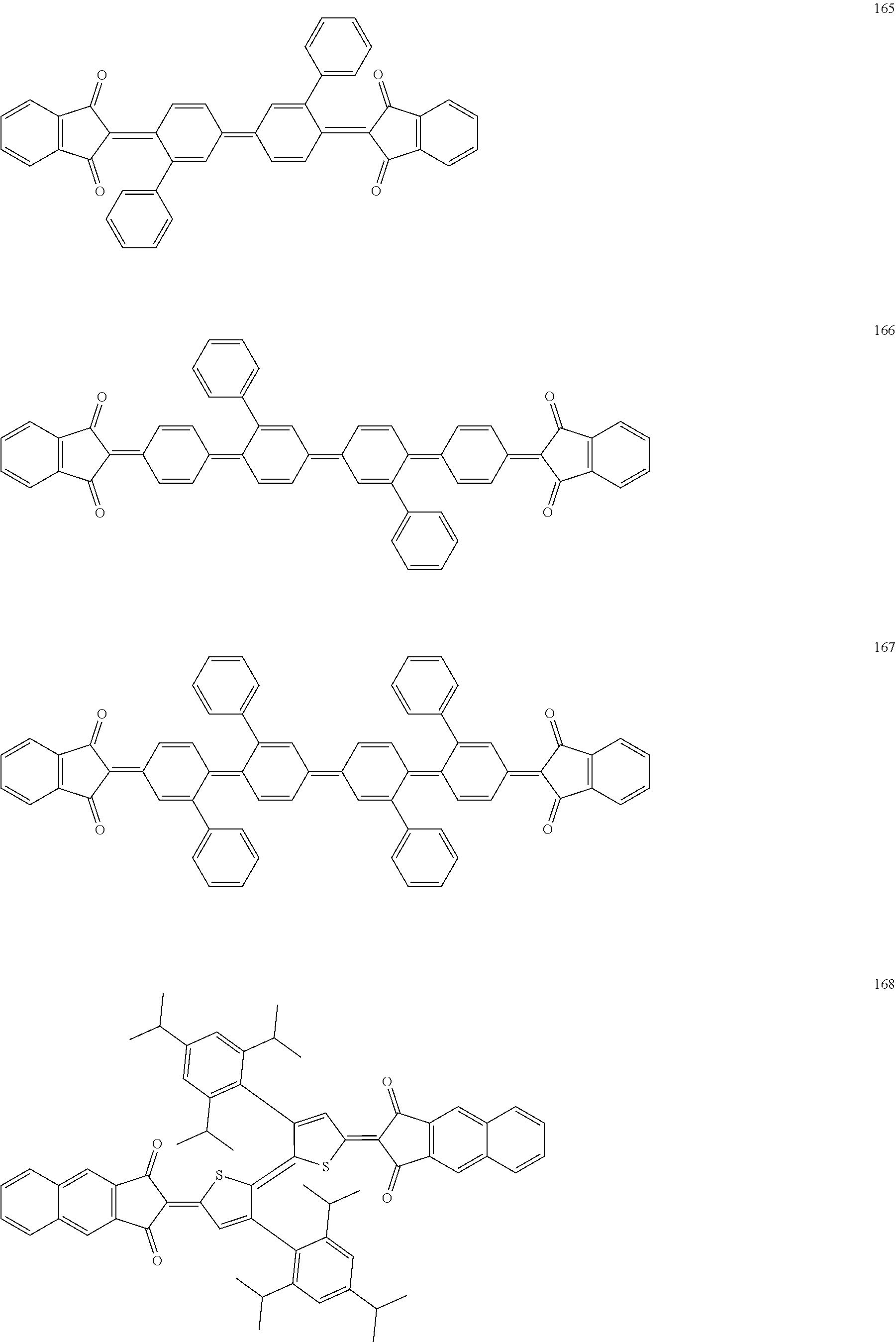

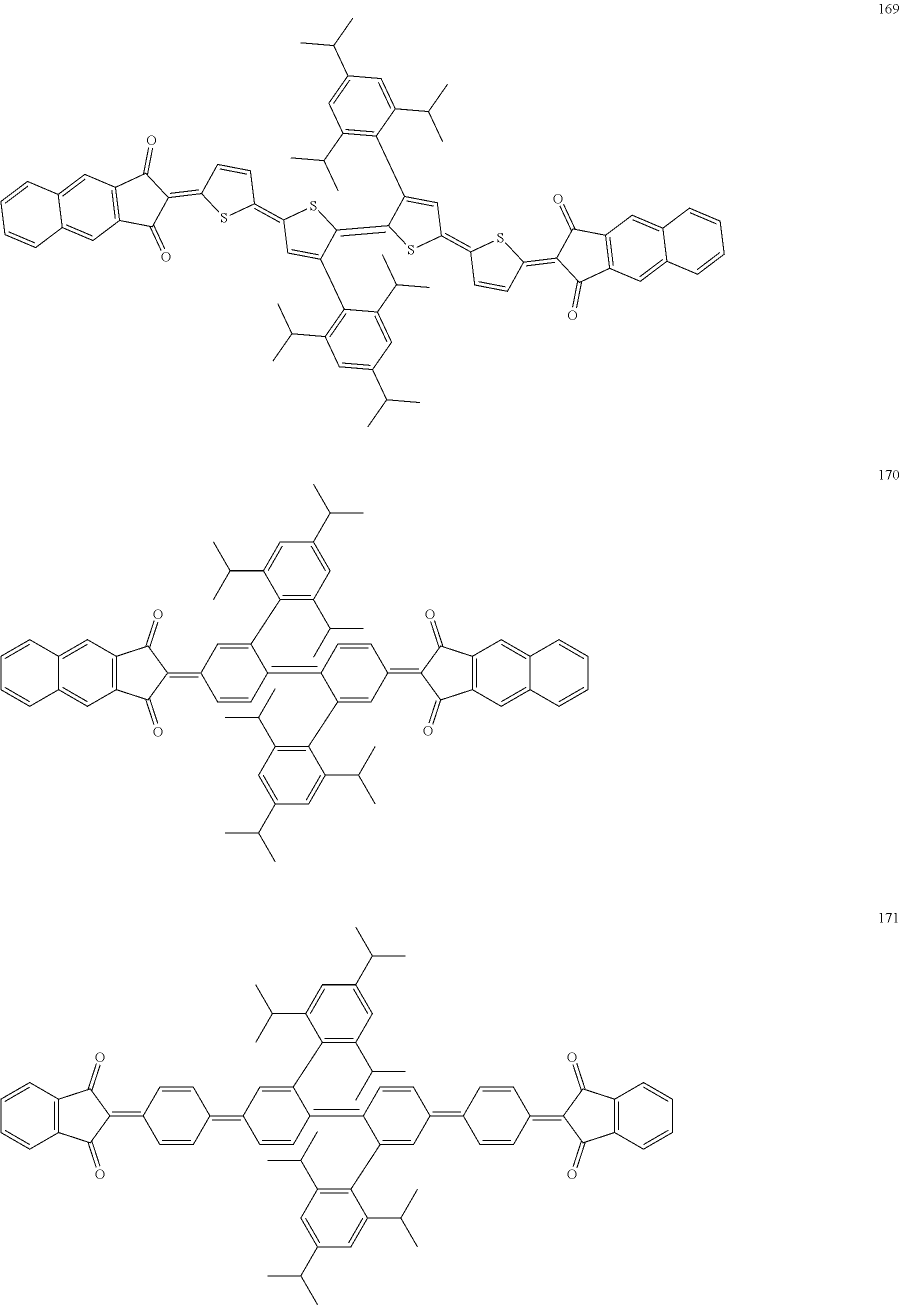 [SMILES: CC(C)c1cc(C(C)C)c(Cc2c/c(=c3/ccc(=C4C(=O)c5cc6ccccc6cc5C4=O)s3)s/c2=c2/s/c(=c3\ccc(=C4C(=O)c5cc6ccccc6cc5C4=O)s3)cc2Cc2c(C(C)C)cc(C(C)C)cc2C(C)C)c(C(C)C)c1 ; CC(C)c1cc(C(C)C)c(-c2cc(=C3C(=O)c4cc5ccccc5cc4C3=O)cc/c2=c2/ccc(=C3C(=O)c4cc5ccccc5cc4C3=O)cc2-c2c(C(C)C)cc(C(C)C)cc2C(C)C)c(C(C)C)c1 ; CC(C)c1cc(C(C)C)c(-c2cc(=c3ccc(=C4C(=O)c5ccccc5C4=O)cc3)cc/c2=c2/ccc(=c3ccc(=C4C(=O)c5ccccc5C4=O)cc3)cc2-c2c(C(C)C)cc(C(C)C)cc2C(C)C)c(C(C)C)c1]

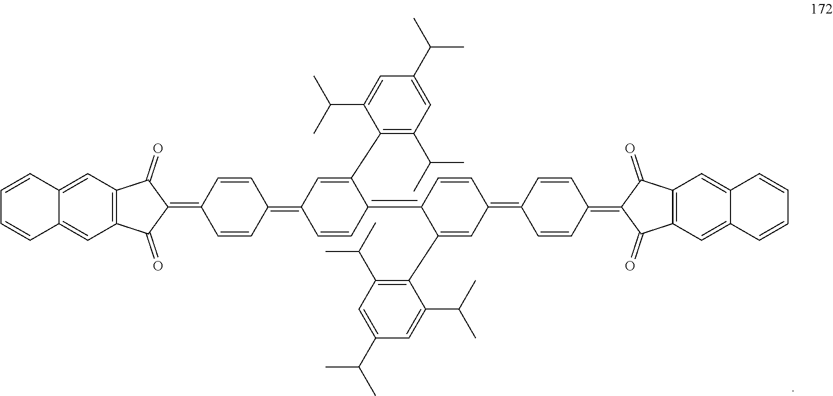 [SMILES: CC(C)c1cc(C(C)C)c(-c2cc(=c3ccc(=C4C(=O)c5cc6ccccc6cc5C4=O)cc3)cc/c2=c2/ccc(=c3ccc(=C4C(=O)c5cc6ccccc6cc5C4=O)cc3)cc2-c2c(C(C)C)cc(C(C)C)cc2C(C)C)c(C(C)C)c1]

The active layer 140 generates excitons in response to light irradiation from the outside and divides the generated excitons into holes and electrons. The active layer 140 may include the compound represented by one selected from Formulae 1 to 4. In addition, the active layer 140 may further include an electron donor and/or an electron acceptor.

In an embodiment, the active layer 140 may include the compound represented by one selected from Formulae 1 to 4 as an electron acceptor or electron donor.

In an embodiment, the active layer 140 may absorb green, red, and/or near-infrared rays. Because the compound represented by one selected from Formulae 1 to 4 has a narrow band gap, the compound may absorb green, red, and/or near-infrared rays. Thus, the active layer 140 including the compound may absorb green, red, and/or near-infrared rays.

The compound represented by one selected from Formulae 1 to 4 may act as an electron acceptor or electron donor. In an embodiment, the active layer 140 may include: the compound represented by one selected from Formulae 1 to 4; and an electron donor or electron acceptor.

For example, the active layer 140 may include: a layer including the compound represented by one selected from Formulae 1 to 4; and a layer including an electron donor or electron acceptor. In this case, the compound represented by one selected from Formulae 1 to 4 may act as the electron acceptor or electron donor.

For example, the layer including the compound represented by one selected from Formulae 1 to 4; and the layer including the electron donor or electron acceptor may be in contact with each other. The layer including the compound represented by one selected from Formulae 1 to 4; and the layer including the electron donor or electron acceptor may be in direct physical contact with each other.

For example, the layer including the compound represented by one selected from Formulae 1 to 4; and the layer including the electron donor or electron acceptor may form a PN junction. Excitons may be efficiently separated into holes and electrons by photo-induced charge separation occurring at an interface between these layers. In addition, because the active layer 140 is separated into the layer including the compound represented by one selected from Formulae 1 to 4; and the layer including the electron donor or electron acceptor, capture and migration of holes and electrons generated at the interface may be facilitated.

In an embodiment, the active layer 140 may include a layer including a mixture of the compound represented by one selected from Formulae 1 to 4; and an electron donor or electron acceptor. In this case, the compound represented by one selected from Formulae 1 to 4 may act as the electron acceptor or electron donor. In this case, the active layer 140 may be formed by co-deposition of the compound represented by one selected from Formulae 1 to 4; and the electron donor or electron acceptor. When the active layer 140 is a mixed layer, excitons may be generated with a diffusion distance from a donor-acceptor interface, and thus, the organic photodetector may have improved external quantum efficiency. A ratio of the compound represented by one selected from Formulae 1 to 4 to the electron donor or electron acceptor may be, for example, in a range of about 10:90 to about 90:10 (weight ratio).

In an embodiment, the active layer 140 may include a layer consisting of the compound represented by one selected from Formulae 1 to 4. In this case, the compound represented by one selected from Formulae 1 to 4 may act as an electron acceptor and/or an electron donor.

In an embodiment, the active layer 140 may include a p-dopant. The p-dopant may be homogeneously or non-homogeneously dispersed in the active layer 140. The active layer 140 is doped with the p-dopant, and thus, external quantum efficiency may be improved by the charge injection principle by an electric field. The p-dopant will be further described below.

In an embodiment, the electron donor may be an organic or inorganic material having a LUMO energy level deeper than −2 eV and a HOMO energy level deeper than −3 eV. In an embodiment, the electron donor may be an organic or inorganic material having a LUMO energy level in a range of about −3 eV to about −5 eV and a HOMO energy level in a range of about −4 eV to about −7 eV. For example, the electron donor may be boron subphthalocyanine chloride (SubPc), copper(II) phthalocyanine (CuPc), tetraphenyldibenzoperiflanthene (DBP), or any combination thereof.

In an embodiment, the electron acceptor may be an organic or inorganic material having a LUMO energy level deeper than −3 eV and a HOMO energy level deeper than −4 eV. For example, the electron acceptor may be an organic or inorganic material having a LUMO energy level in a range of about −4 eV to about −6 eV and a HOMO energy level in a range of about −5 eV to about −8 eV. For example, the electron acceptor may be C60 fullerene, HATCN, TCNQ, and/or a diimide-based nonfullerene.

As a diimide-based nonfullerene compound, for example, a compound represented by Formula 5 may be used:

### Formula 5

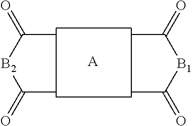 [0094] wherein, in Formula 5,
A may be a C₃-C₆₀ carbocyclic group unsubstituted or substituted with at least one R₁₀ₐ or a C₁-C₆₀ heterocyclic group unsubstituted or substituted with at least one R₁₀ₐ,
B₁ and B₂ may each independently be oxygen or —NR₁₀—, wherein R₁₀ may be hydrogen, a C₁-C₆₀ alkyl group unsubstituted or substituted with at least one R₁₀ₐ, a C₃-C₁₀ cycloalkyl group unsubstituted or substituted with at least one R₁₀ₐ, a halogen atom, or a halogen-containing group.

Examples of the compound represented by Formula 5 are as follows, but are not limited thereto:

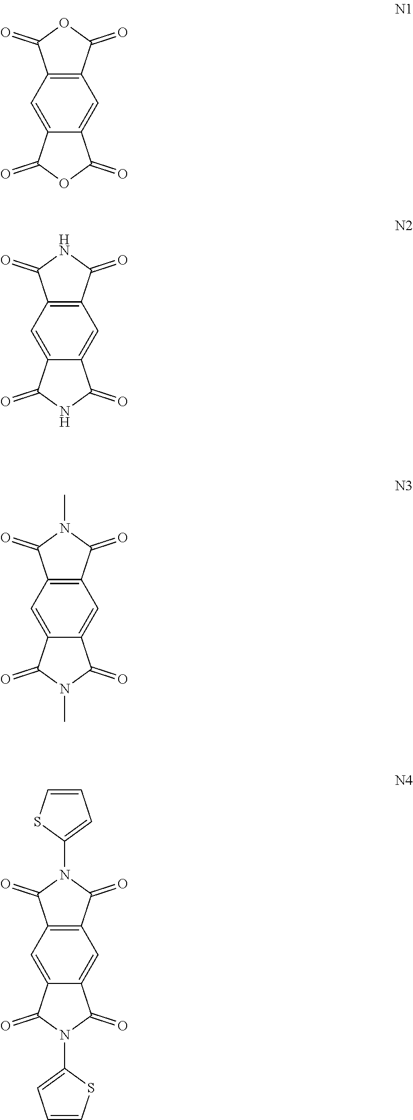 [SMILES: O=c1oc(=O)c2cc3c(=O)oc(=O)c3cc12 ; O=c1[nH]c(=O)c2cc3c(=O)[nH]c(=O)c3cc12 ; Cn1c(=O)c2cc3c(=O)n(C)c(=O)c3cc2c1=O ; O=c1c2cc3c(=O)n(-c4cccs4)c(=O)c3cc2c(=O)n1-c1cccs1]

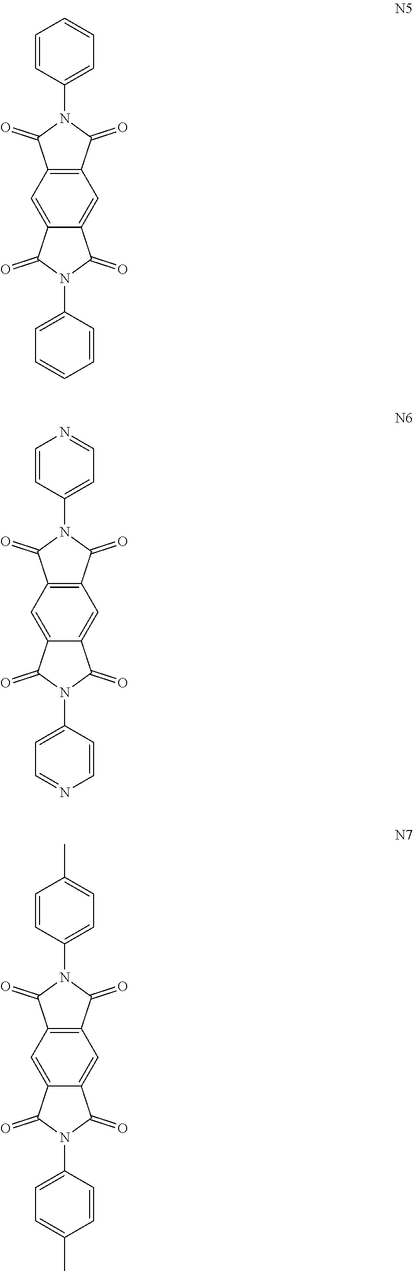 [SMILES: O=c1c2cc3c(=O)n(-c4ccccc4)c(=O)c3cc2c(=O)n1-c1ccccc1 ; O=c1c2cc3c(=O)n(-c4ccncc4)c(=O)c3cc2c(=O)n1-c1ccncc1 ; Cc1ccc(-n2c(=O)c3cc4c(=O)n(-c5ccc(C)cc5)c(=O)c4cc3c2=O)cc1]

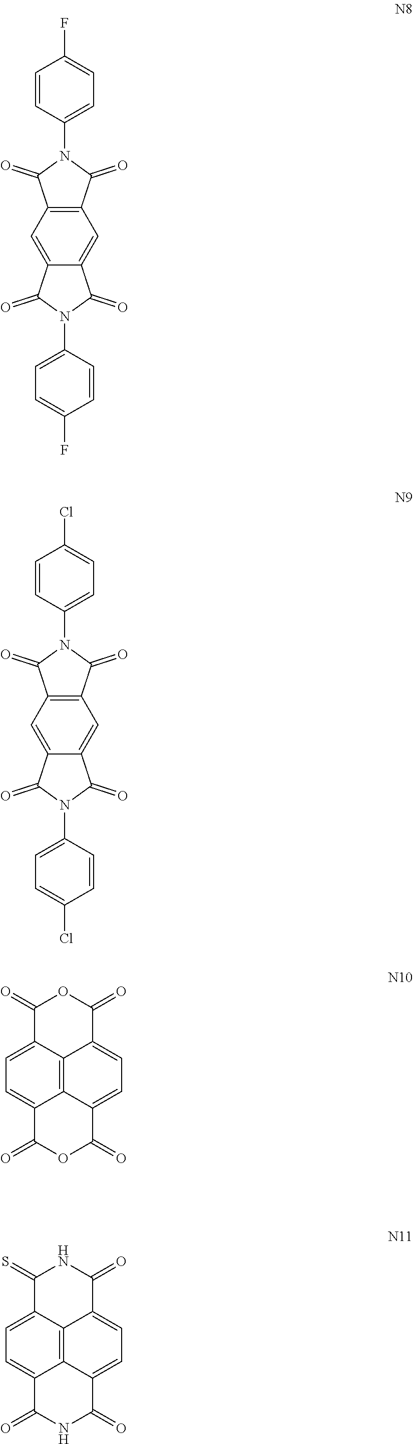 [SMILES: O=c1c2cc3c(=O)n(-c4ccc(F)cc4)c(=O)c3cc2c(=O)n1-c1ccc(F)cc1 ; O=c1c2cc3c(=O)n(-c4ccc(Cl)cc4)c(=O)c3cc2c(=O)n1-c1ccc(Cl)cc1 ; O=C1OC(=O)c2ccc3c4c(ccc1c24)C(=O)OC3=O ; O=C1NC(=O)c2ccc3c4c(ccc1c24)C(=O)NC3=S]

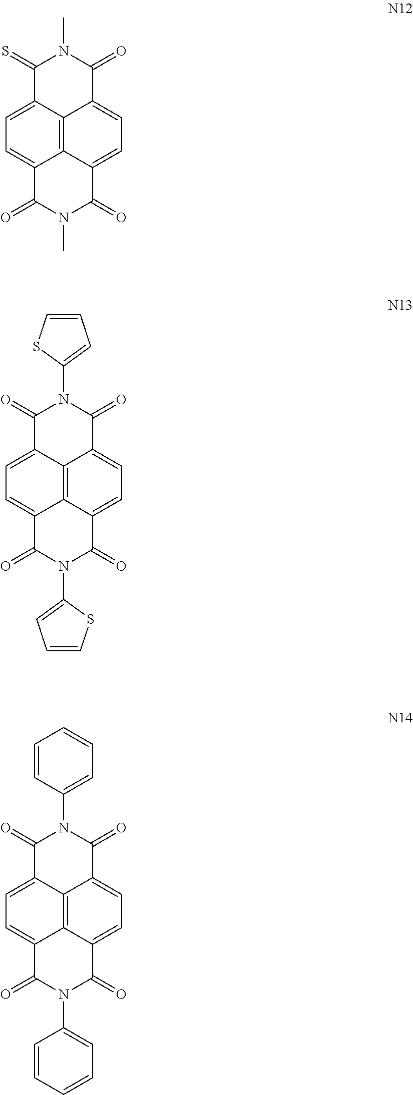 [SMILES: CN1C(=O)c2ccc3c4c(ccc(c24)C1=O)C(=S)N(C)C3=O ; O=C1c2ccc3c4c(ccc(c24)C(=O)N1c1cccs1)C(=O)N(c1cccs1)C3=O ; O=C1c2ccc3c4c(ccc(c24)C(=O)N1c1ccccc1)C(=O)N(c1ccccc1)C3=O]

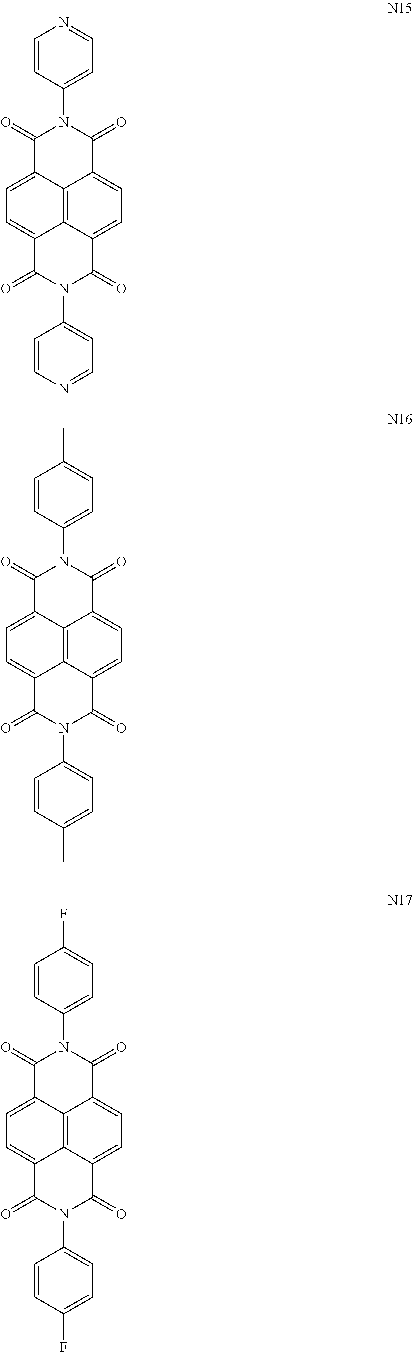

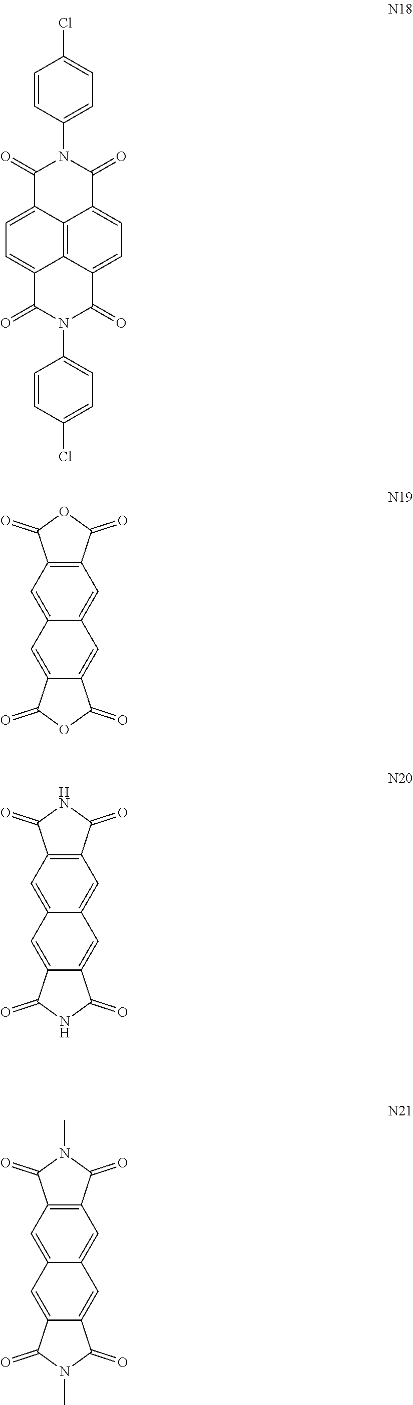 [SMILES: O=C1c2ccc3c4c(ccc(c24)C(=O)N1c1ccc(Cl)cc1)C(=O)N(c1ccc(Cl)cc1)C3=O ; O=c1oc(=O)c2cc3cc4c(=O)oc(=O)c4cc3cc12 ; O=c1[nH]c(=O)c2cc3cc4c(=O)[nH]c(=O)c4cc3cc12 ; Cn1c(=O)c2cc3cc4c(=O)n(C)c(=O)c4cc3cc2c1=O]

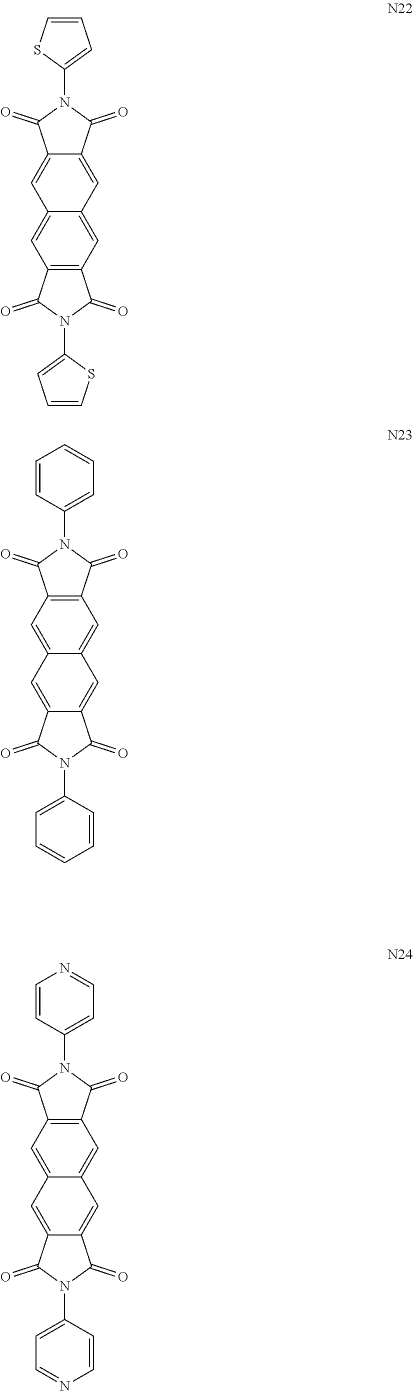

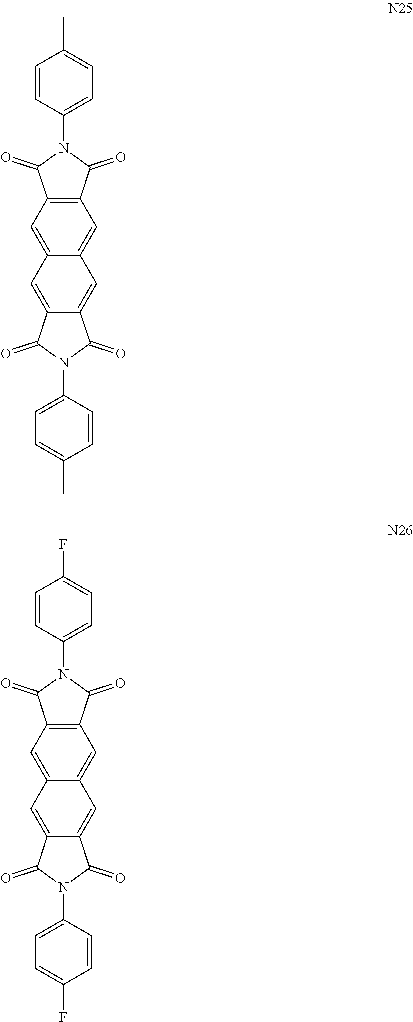 [SMILES: Cc1ccc(-n2c(=O)c3cc4cc5c(=O)n(-c6ccc(C)cc6)c(=O)c5cc4cc3c2=O)cc1 ; O=c1c2cc3cc4c(=O)n(-c5ccc(F)cc5)c(=O)c4cc3cc2c(=O)n1-c1ccc(F)cc1]

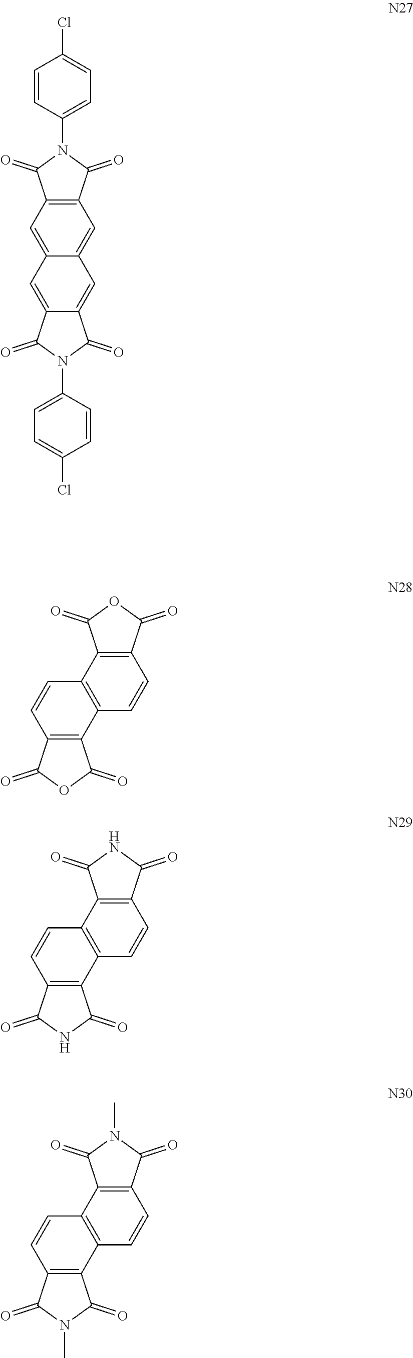 [SMILES: O=c1c2cc3cc4c(=O)n(-c5ccc(Cl)cc5)c(=O)c4cc3cc2c(=O)n1-c1ccc(Cl)cc1 ; O=c1oc(=O)c2c1ccc1c2ccc2c(=O)oc(=O)c21 ; O=c1[nH]c(=O)c2c1ccc1c2ccc2c(=O)[nH]c(=O)c21 ; Cn1c(=O)c2ccc3c(ccc4c(=O)n(C)c(=O)c43)c2c1=O]

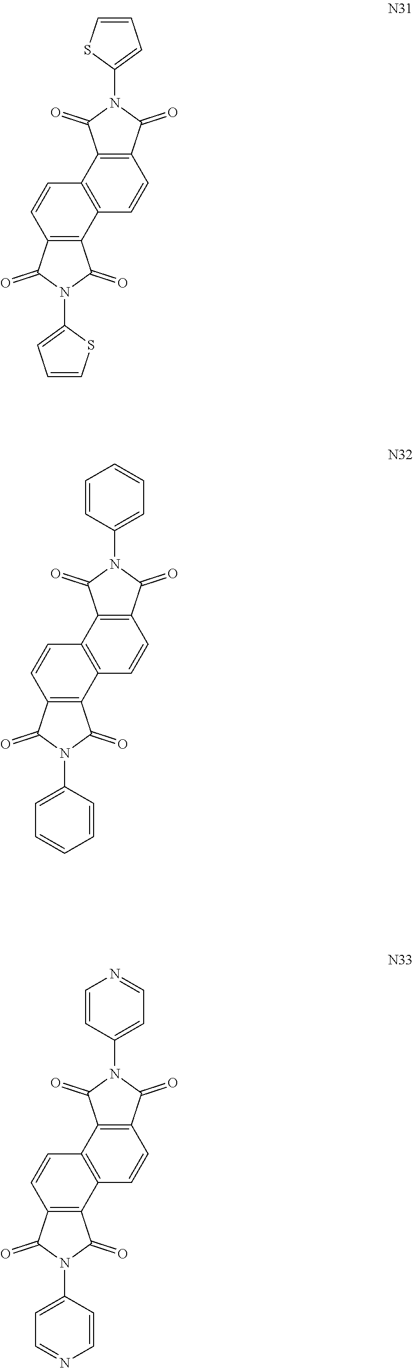 [SMILES: O=c1c2ccc3c(ccc4c(=O)n(-c5cccs5)c(=O)c43)c2c(=O)n1-c1cccs1 ; O=c1c2ccc3c(ccc4c(=O)n(-c5ccccc5)c(=O)c43)c2c(=O)n1-c1ccccc1 ; O=c1c2ccc3c(ccc4c(=O)n(-c5ccncc5)c(=O)c43)c2c(=O)n1-c1ccncc1]

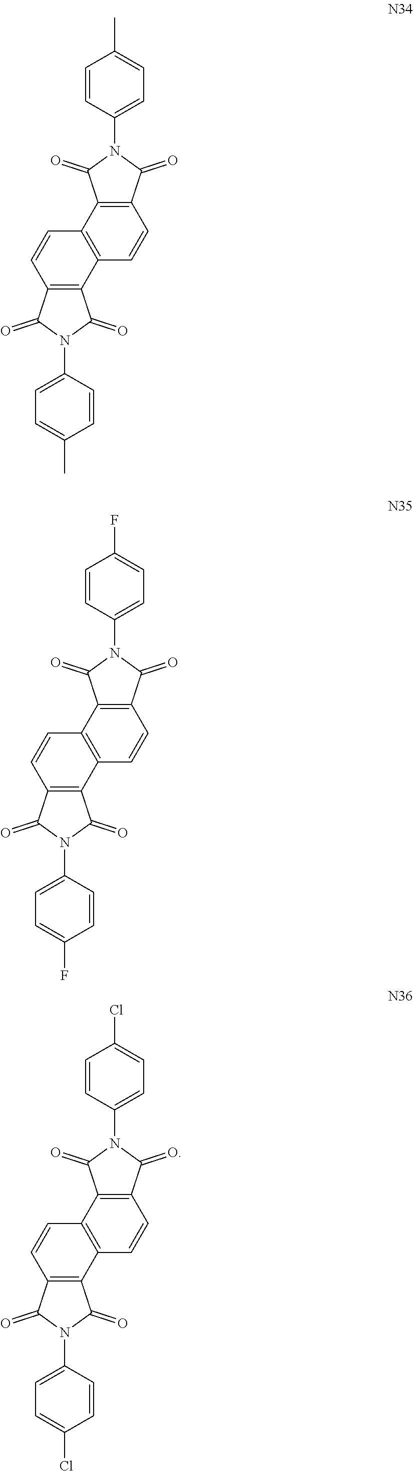 [SMILES: Cc1ccc(-n2c(=O)c3ccc4c(ccc5c(=O)n(-c6ccc(C)cc6)c(=O)c54)c3c2=O)cc1 ; O=c1c2ccc3c(ccc4c(=O)n(-c5ccc(F)cc5)c(=O)c43)c2c(=O)n1-c1ccc(F)cc1 ; O=c1c2ccc3c(ccc4c(=O)n(-c5ccc(Cl)cc5)c(=O)c43)c2c(=O)n1-c1ccc(Cl)cc1]

One selected from the first electrode 110 and the second electrode 170 may be an anode, and the other one may be a cathode. For example, the first electrode 110 may be an anode, and the second electrode 170 may be a cathode.

A hole transport region of the organic photodetector 10 may include a structure in which the hole injection layer 120, the hole transport layer 130, an auxiliary layer, an electron-blocking layer, or any combination thereof are on the first electrode 110. For example, the auxiliary layer may be between the hole transport layer 130 and the active layer 140.

An electron transport region of the organic photodetector 10 may include a structure in which a buffer layer, a hole-blocking layer, the electron transport layer 150, the electron injection layer 160, or any combination thereof are on the active layer 140. For example, the buffer layer may be located between the electron transport layer 150 and the active layer 140.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, a compound represented by Formula 221, and the like:

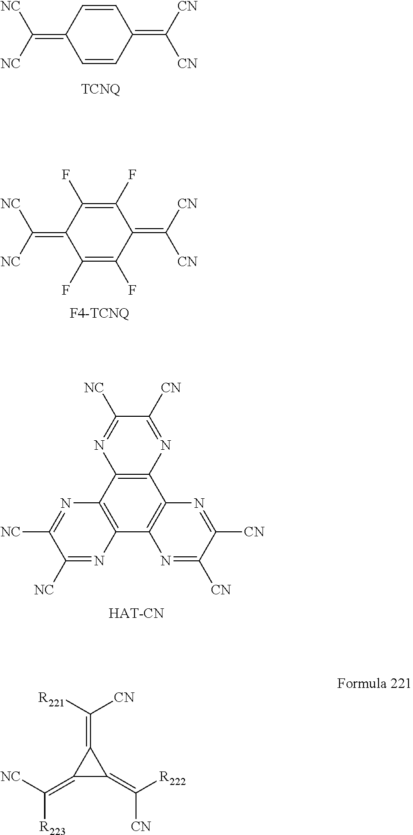

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from R₂₂₁ to R₂₂₃ may each independently be: a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Examples of the metal may include: an alkali metal (e.g., lithium (Li), sodium (Na), potassium (K), rubidium (Rb), cesium (Cs), etc.); an alkaline earth metal (e.g., beryllium (Be), magnesium (Mg), calcium (Ca), strontium (Sr), barium (Ba), etc.); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (e.g., zinc (Zn), indium (In), tin (Sn), etc.); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and the like.

Examples of the metalloid may include silicon (Si), antimony (Sb), tellurium (Te), and the like.

Examples of the non-metal may include oxygen (O), halogen (e.g., F, Cl, Br, I, etc.), and the like.

Examples of the compound containing element EL1 and element EL2 may include a metal oxide, a metal halide (e.g., a metal fluoride, a metal chloride, a metal bromide, a metal iodide, etc.), a metalloid halide (e.g., a metalloid fluoride, a metalloid chloride, a metalloid bromide, a metalloid iodide, etc.), a metal telluride, or any combination thereof.

Examples of the metal oxide may include a tungsten oxide (e.g., WO, W₂O₃, WO₂, WO₃, W₂O₅, etc.), a vanadium oxide (e.g., VO, V₂O₃, VO₂, V₂O₅, etc.), a molybdenum oxide (e.g., MoO, Mo₂O₃, MoO₂, MoO₃, Mo₂O₅, etc.), a rhenium oxide (e.g., ReO₃, etc.), and the like.

Examples of the metal halide may include an alkali metal halide, an alkaline earth metal halide, a transition metal halide, a post-transition metal halide, a lanthanide metal halide, and the like.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, BaI₂, and the like.

Examples of the transition metal halide may include a titanium halide (e.g., TiF₄, TiCl₄, TiBr₄, TiI₄, etc.), a zirconium halide (e.g., ZrF₄, ZrCl₄, ZrBr₄, ZrI₄, etc.), a hafnium halide (e.g., HfF₄, HfCl₄, HfBr₄, HfI₄, etc.), a vanadium halide (e.g., VF₃, VCl₃, VBr₃, VI₃, etc.), a niobium halide (e.g., NbF₃, NbCl₃, NbBr₃, NbI₃, etc.), a tantalum halide (e.g., TaF₃, TaCl₃, TaBr₃, TaI₃, etc.), a chromium halide (e.g., CrF₃, CrCl₃, CrBr₃, CrI₃, etc.), a molybdenum halide (e.g., MoF₃, MoCl₃, MoBr₃, MoI₃, etc.), a tungsten halide (e.g., WF₃, WCl₃, WBr₃, WI₃, etc.), a manganese halide (e.g., MnF₂, MnCl₂, MnBr₂, MnI₂, etc.), a technetium halide (e.g., TcF₂, TcCl₂, TcBr₂, TcI₂, etc.), a rhenium halide (e.g., ReF₂, ReCl₂, ReBr₂, ReI₂, etc.), an iron halide (e.g., FeF₂, FeCl₂, FeBr₂, FeI₂, etc.), a ruthenium halide (e.g., RuF₂, RuCl₂, RuBr₂, RuI₂, etc.), an osmium halide (e.g., OsF₂, OsCl₂, OsBr₂, OsI₂, etc.), a cobalt halide (e.g., CoF₂, CoCl₂, CoBr₂, CoI₂, etc.), a rhodium halide (e.g., RhF₂, RhCl₂, RhBr₂, RhI₂, etc.), an iridium halide (e.g., IrF₂, IrCl₂, IrBr₂, IrI₂, etc.), a nickel halide (e.g., NiF₂, NiCl₂, NiBr₂, NiI₂, etc.), a palladium halide (e.g., PdF₂, PdCl₂, PdBr₂, PdI₂, etc.), a platinum halide (e.g., PtF₂, PtCl₂, PtBr₂, PtI₂, etc.), a copper halide (e.g., CuF, CuCl, CuBr, CuI, etc.), a silver halide (e.g., AgF, AgCl, AgBr, AgI, etc.), a gold halide (e.g., AuF, AuCl, AuBr, AuI, etc.), and the like.

Examples of the post-transition metal halide may include a zinc halide (e.g., ZnF₂, ZnCl₂, ZnBr₂, ZnI₂, etc.), an indium halide (e.g., InI₃, etc.), a tin halide (e.g., SnI₂, etc.), and the like.

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and the like.

Examples of the metalloid halide may include an antimony halide (e.g., SbCl₅, etc.) and the like.

Examples of the metal telluride may include an alkali metal telluride (e.g., Li₂Te, Na₂Te, K₂Te, Rb₂Te, Cs₂Te, etc.), an alkaline earth metal telluride (e.g., BeTe, MgTe, CaTe, SrTe, BaTe, etc.), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (e.g., ZnTe, etc.), a lanthanide metal telluride (e.g., LaTe, CeTe, PrTe, NdTe, PmTe, EuTe, GdTe, TbTe, DyTe, HoTe, ErTe, TmTe, YbTe, LuTe, etc.), and the like.

In an embodiment, an amount of the p-dopant in the active layer 140 may be in a range of about 0.1 vol % to about 10 vol %, for example, about 0.5 vol % to about 5 vol %.

A thickness of the active layer 140 may be in a range of about 200 Å to about 2,000 Å, for example, about 400 Å to about 600 Å.

### First Electrode 110

In FIG. 1, a substrate may be additionally under the first electrode 110 and/or on the second electrode 170. As the substrate, a glass substrate and/or a plastic substrate may be used. In one or more embodiments, the substrate may be a flexible substrate, and may include plastics having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing and/or sputtering a material for forming the first electrode 110 on the substrate. When the first electrode 110 is an anode, the material for forming the first electrode 110 may be a high-work function material.

The first electrode 110 may be a reflective electrode, a semi-transmissive electrode, or a transmissive electrode. When the first electrode 110 is a transmissive electrode, the material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, the material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Charge Auxiliary Layer

The organic photodetector 10 according to an embodiment may include a charge auxiliary layer that facilitates migration of holes and electrons from the active layer 140.

The charge auxiliary layer may include the hole injection layer 120 and the hole transport layer 130, which facilitate migration of holes, and may include the electron transport layer 150 and the electron injection layer 160, which facilitate migration of electrons.

### Hole Transport Region

The charge auxiliary layers between the first electrode 110 and the active layer 140 may be collectively referred to as a hole transport region.

The hole transport region may include the hole injection layer 120, the hole transport layer 130, the auxiliary layer, and the electron-blocking layer.

The hole transport region may include a hole transport material. For example, the hole transport material may include a compound represented by Formula 201, a compound represented by Formula 202, or any combination thereof:

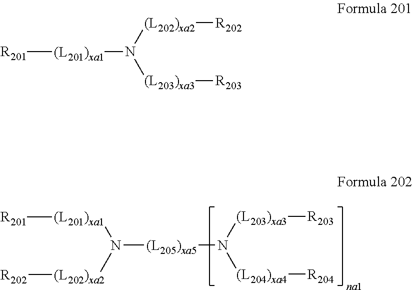

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—*, *—N(Q₂₀₁)*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group, etc.) unsubstituted or substituted with at least one R₁₀ₐ (e.g., Compound HT16, etc.),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

For example, each of Formulae 201 and 202 may include at least one selected from groups represented by Formulae CY201 to CY217:

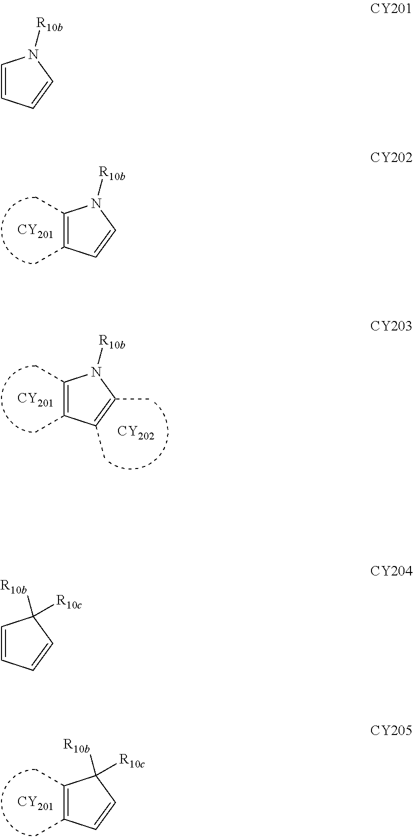

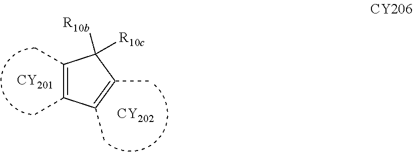

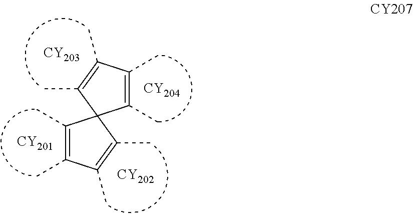

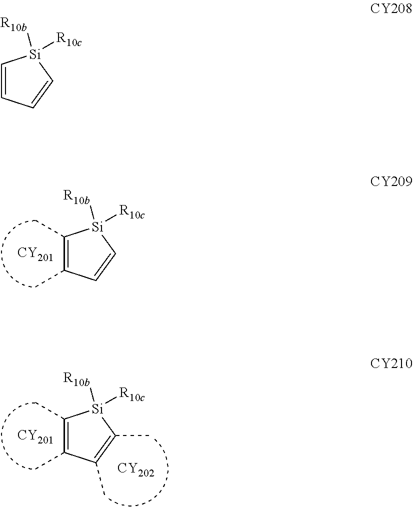

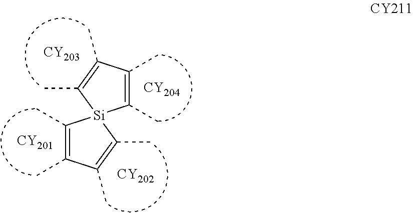

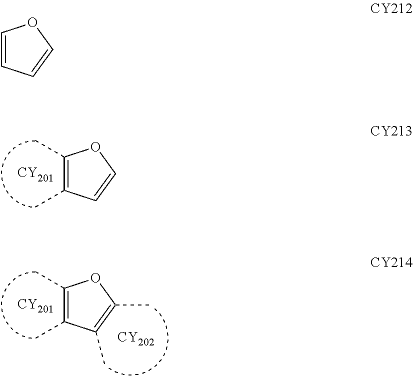

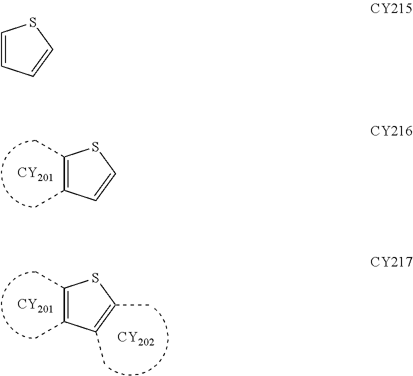

wherein, in Formulae CY201 to CY217, R_{10b} and R_{10c} may each be as defined herein in connection with R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In an embodiment, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from groups represented by Formulae CY201 to CY203 and at least one selected from groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include a group represented by one selected from Formulae CY201 to CY203.

In one or more embodiments, each of Formulae 201 and 202 may not include a group represented by one selected from Formulae CY201 to CY203, and may include at least one selected from groups represented by Formulae CY204 to CY217.

In one or more embodiments, each of Formulae 201 and 202 may not include a group represented by one selected from Formulae CY201 to CY217.

For example, the hole transport material may include one selected from Compounds HT1 to HT46, m-MTDATA, TDATA, 2-TNATA, NPB (NPD), β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

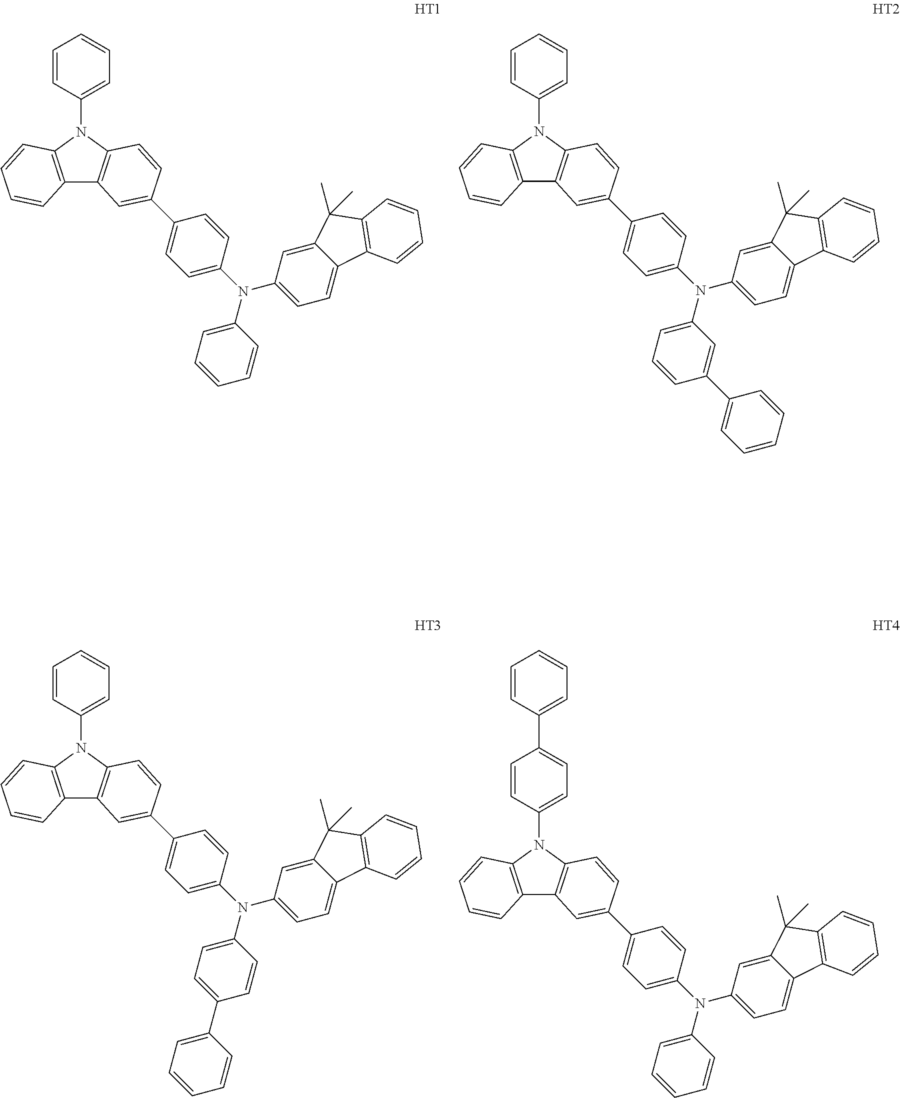

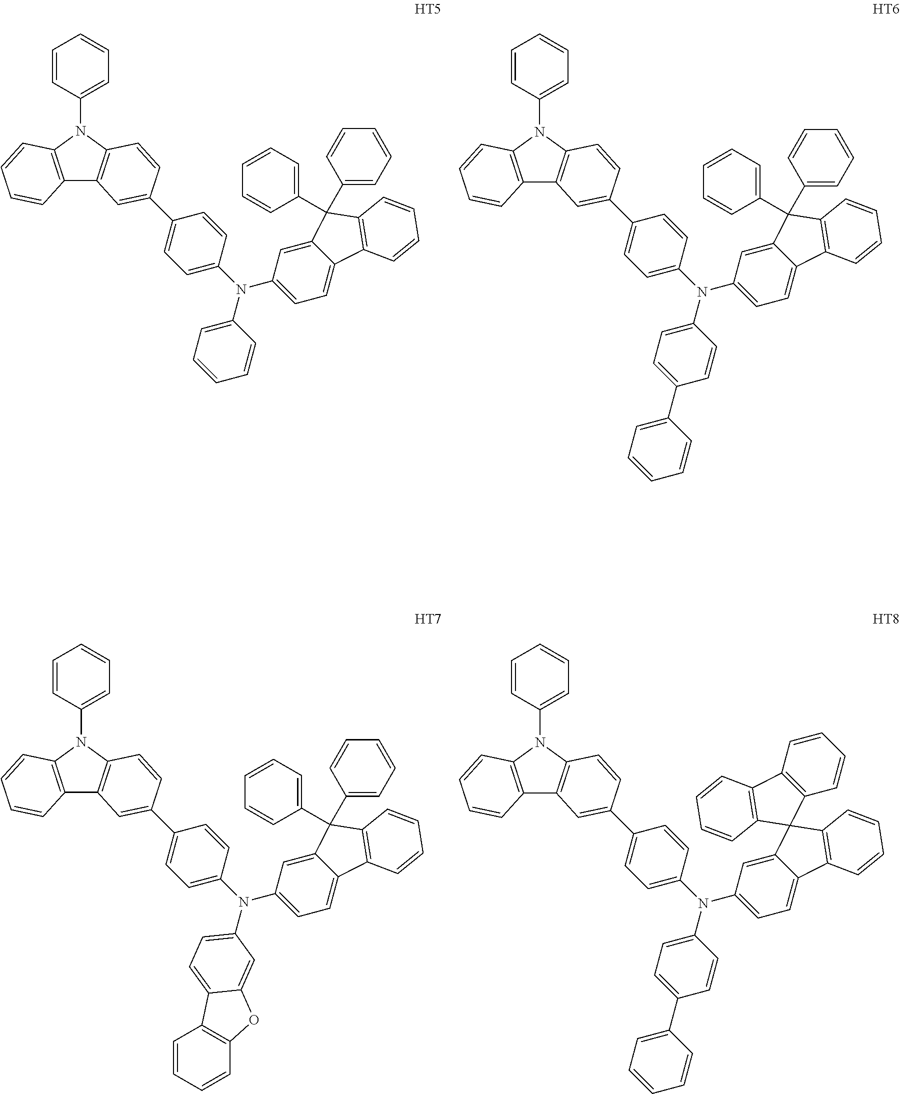 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

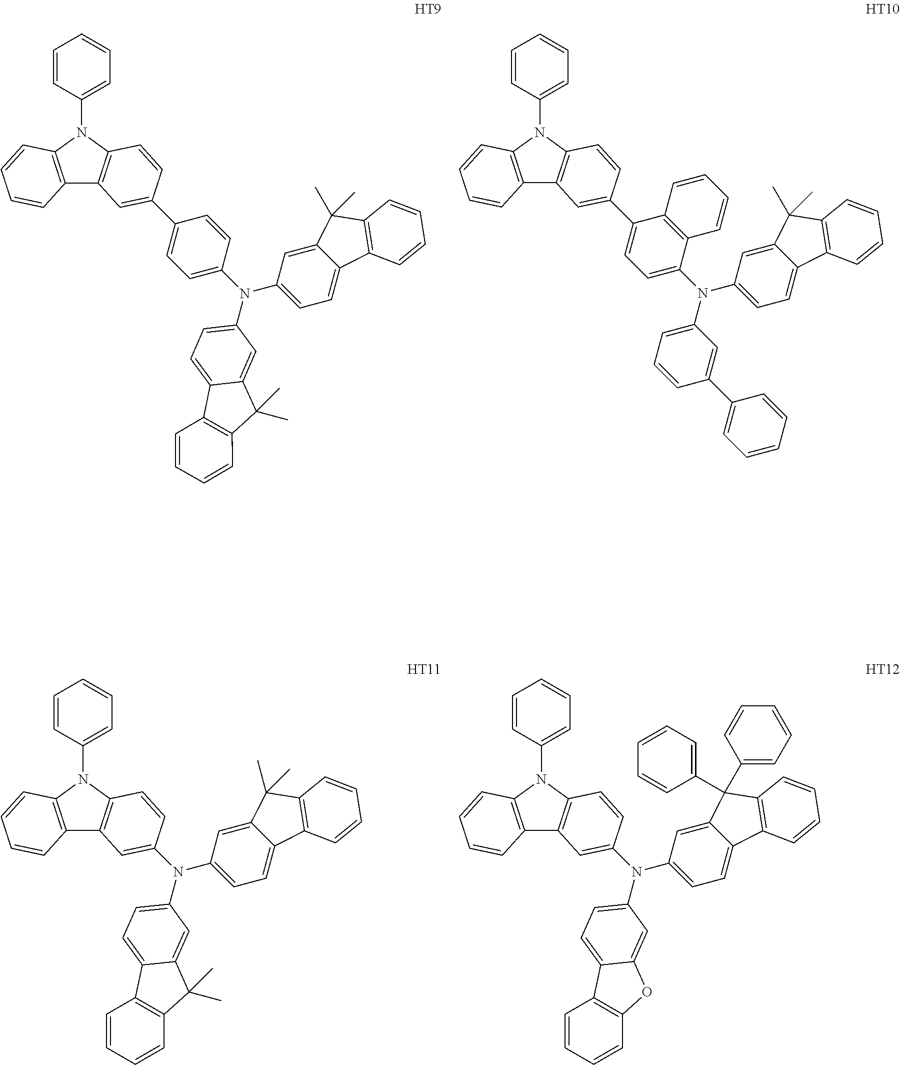 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

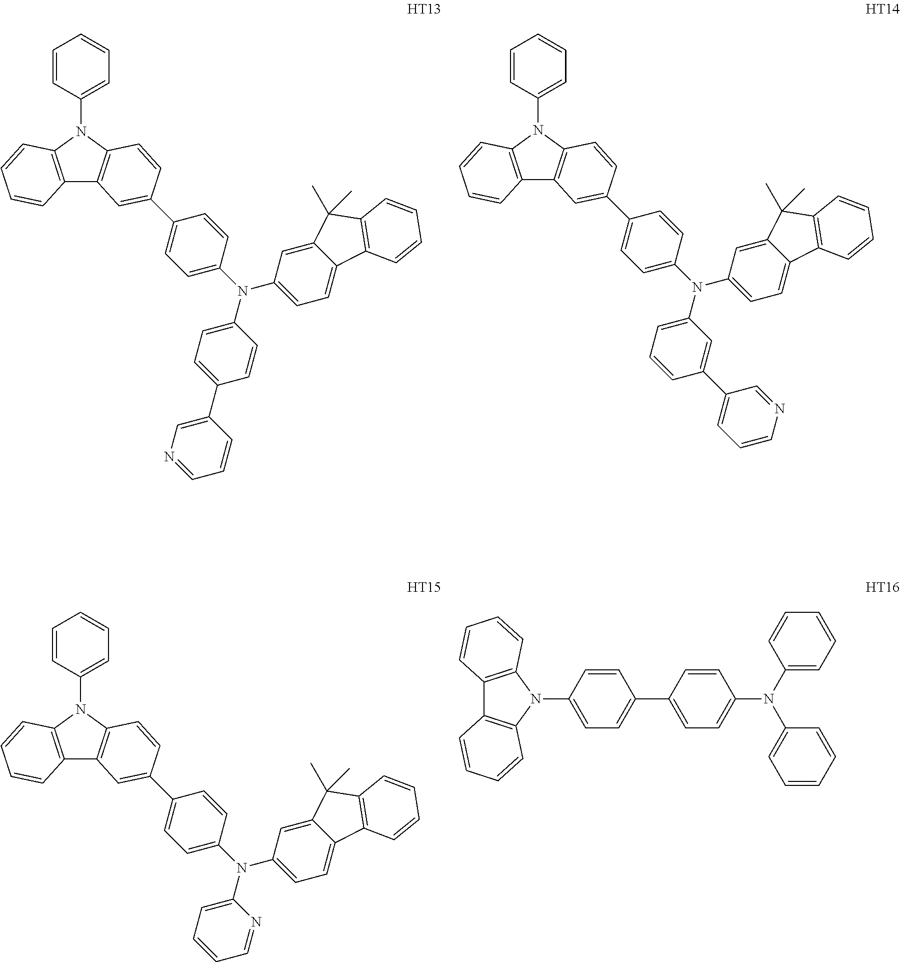 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

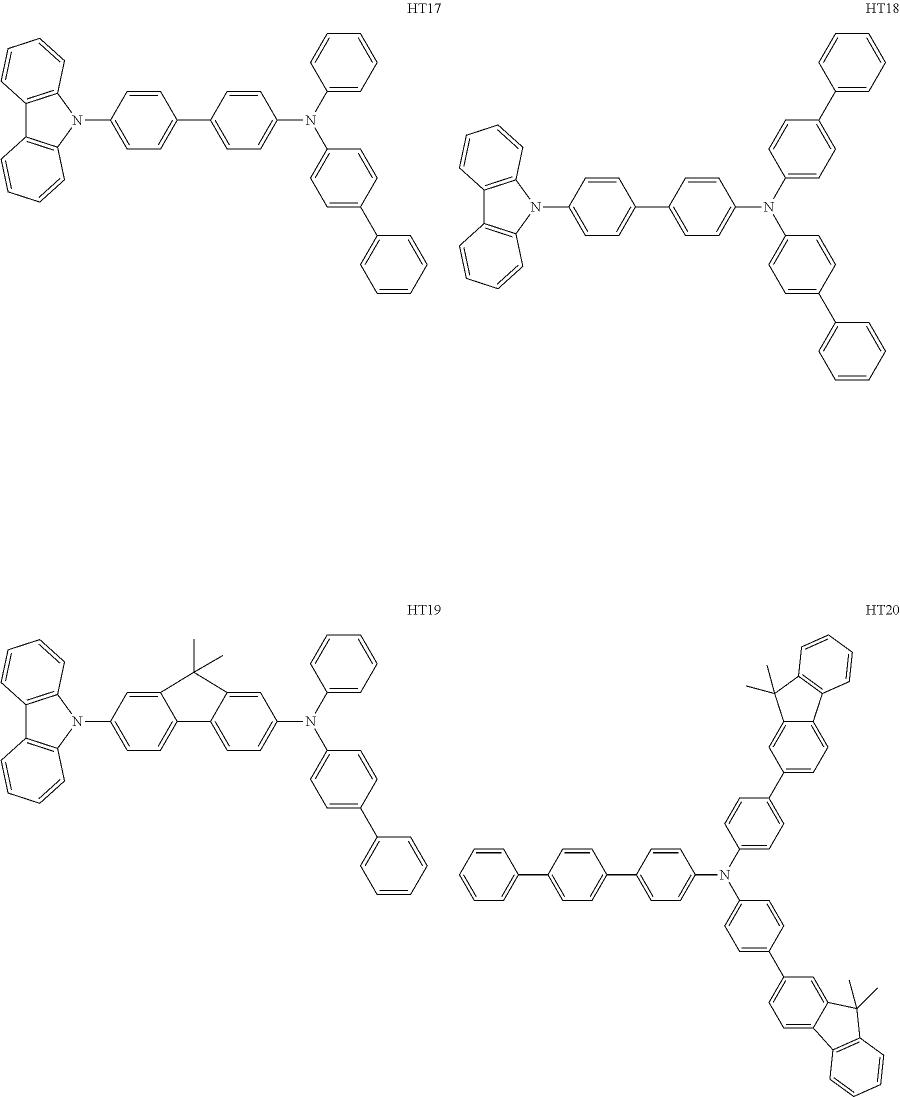

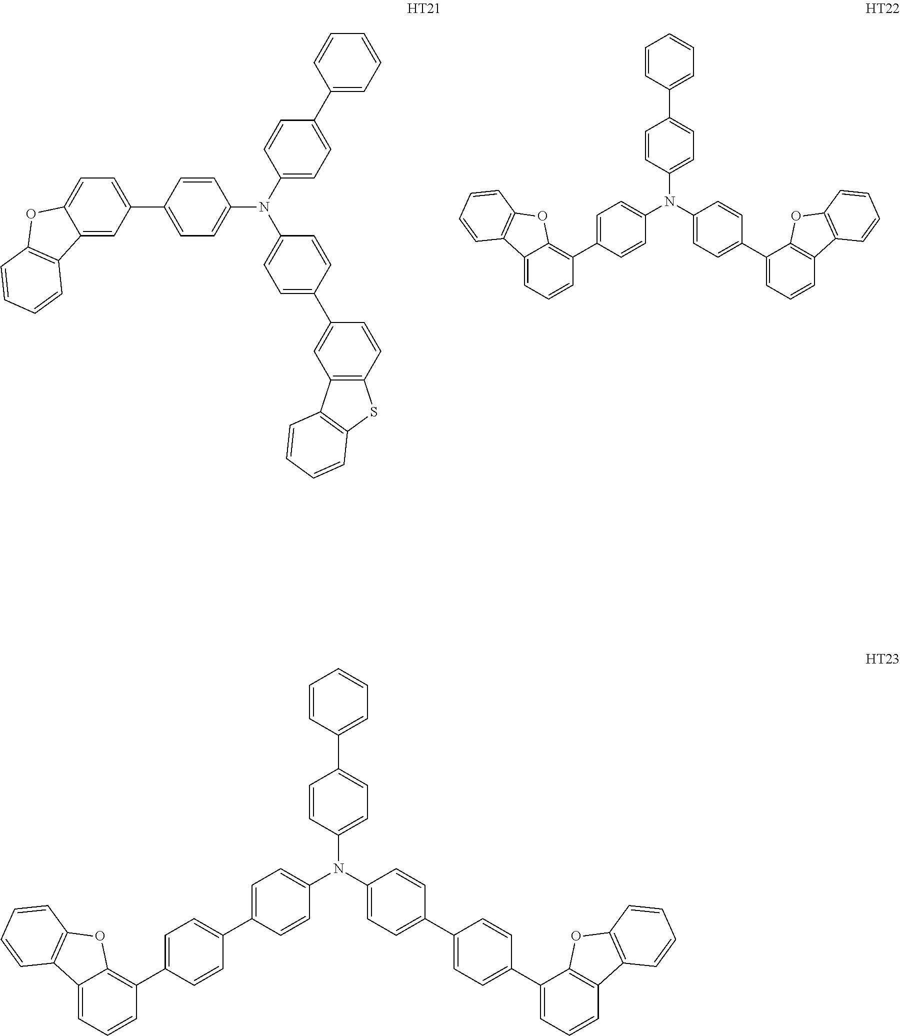 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

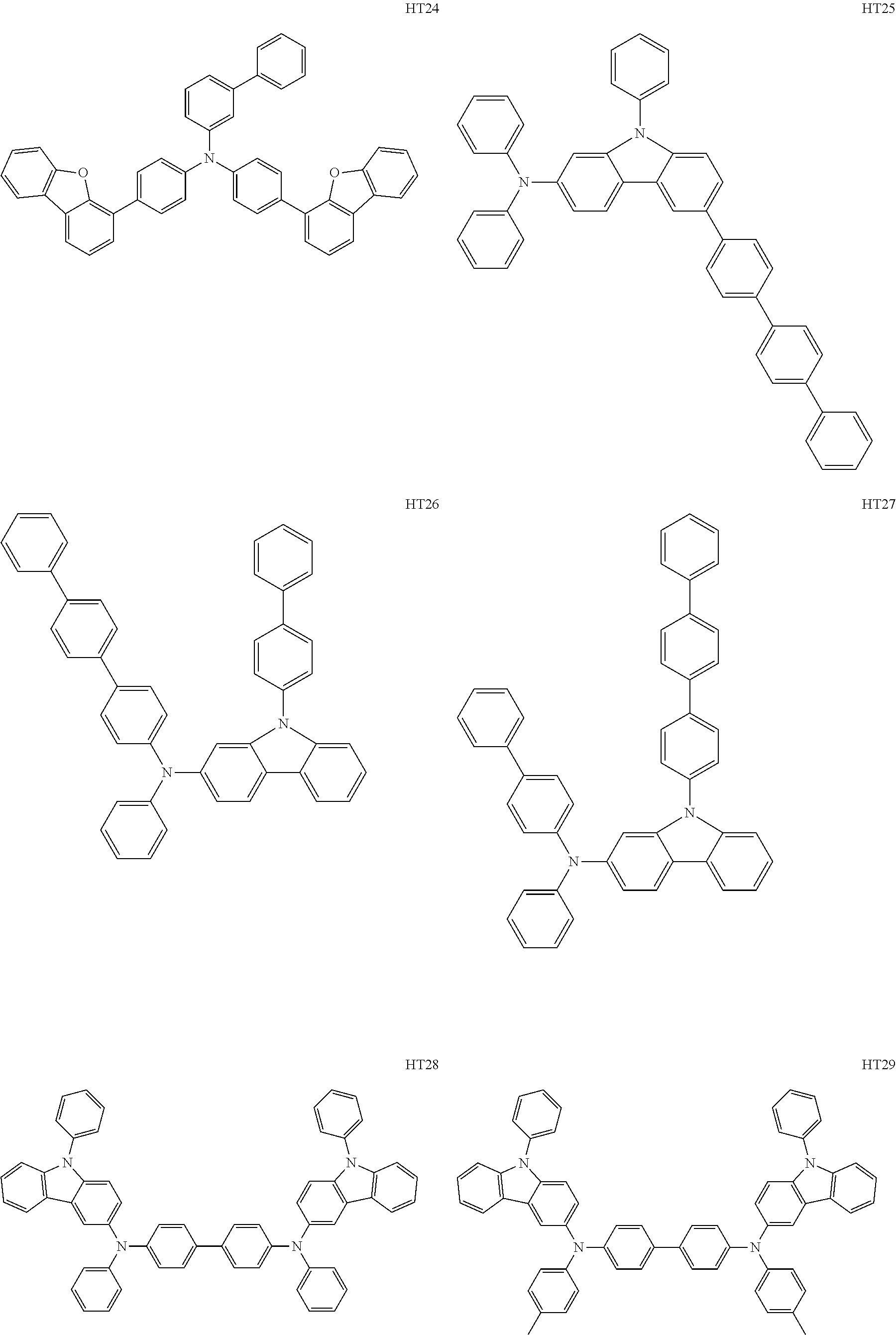

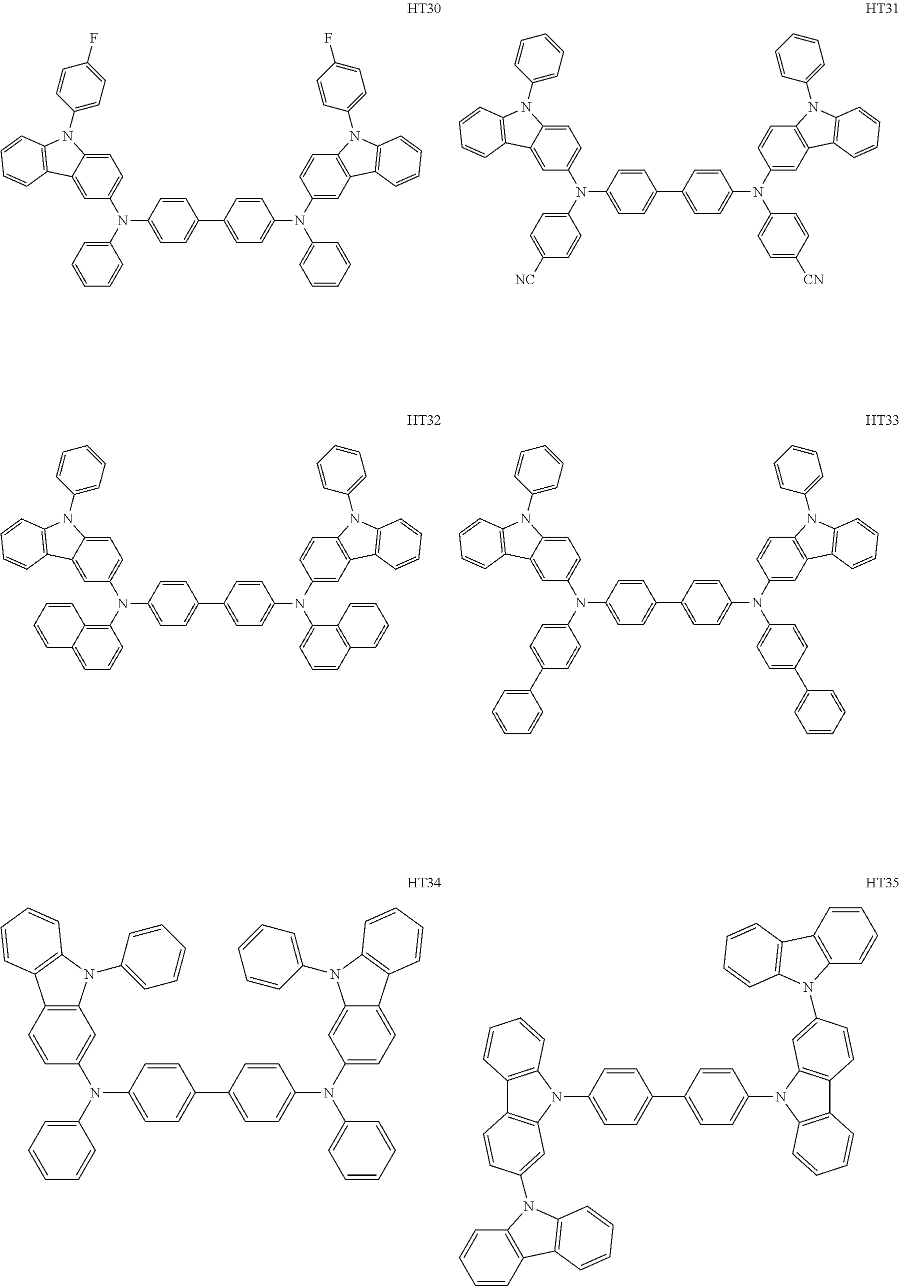

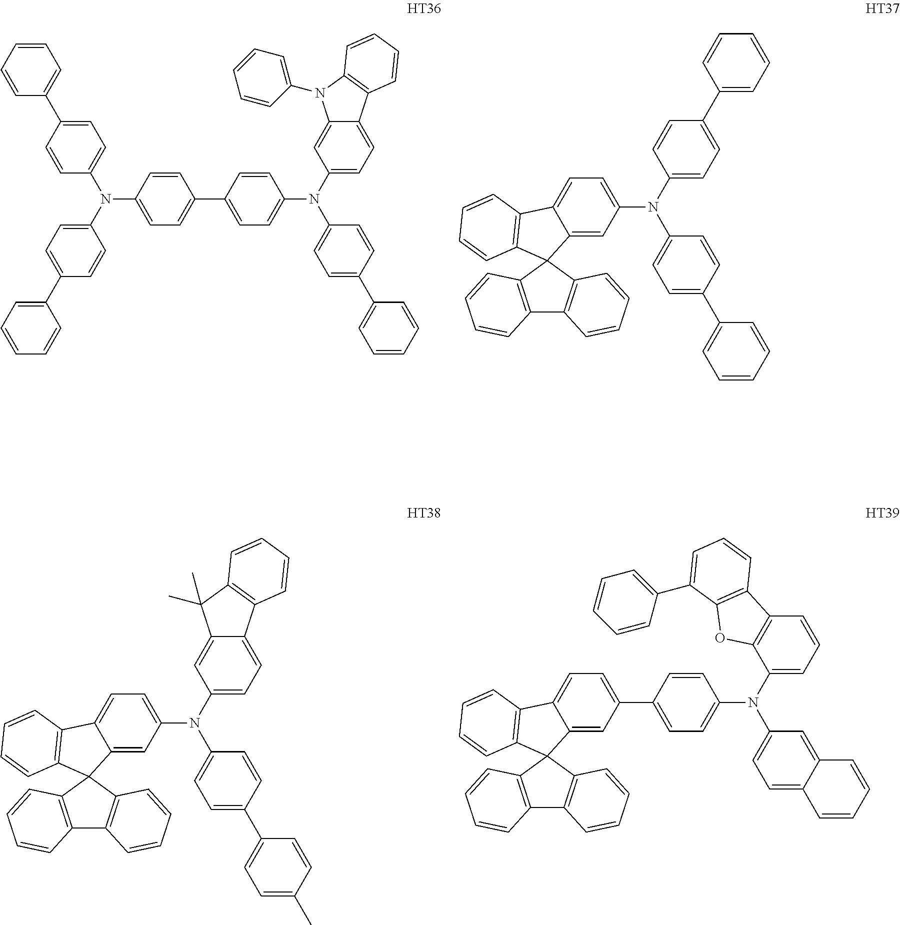 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

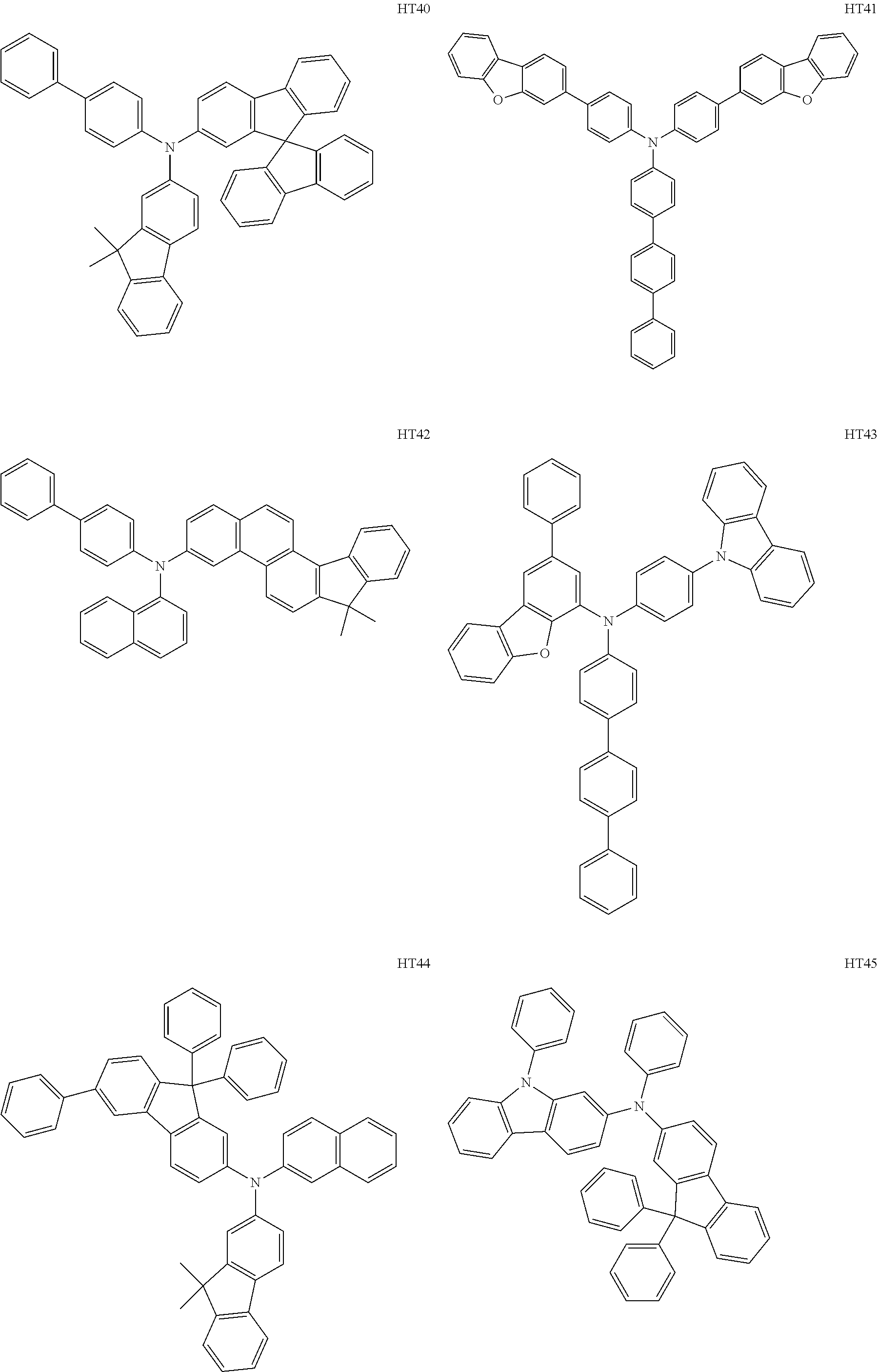

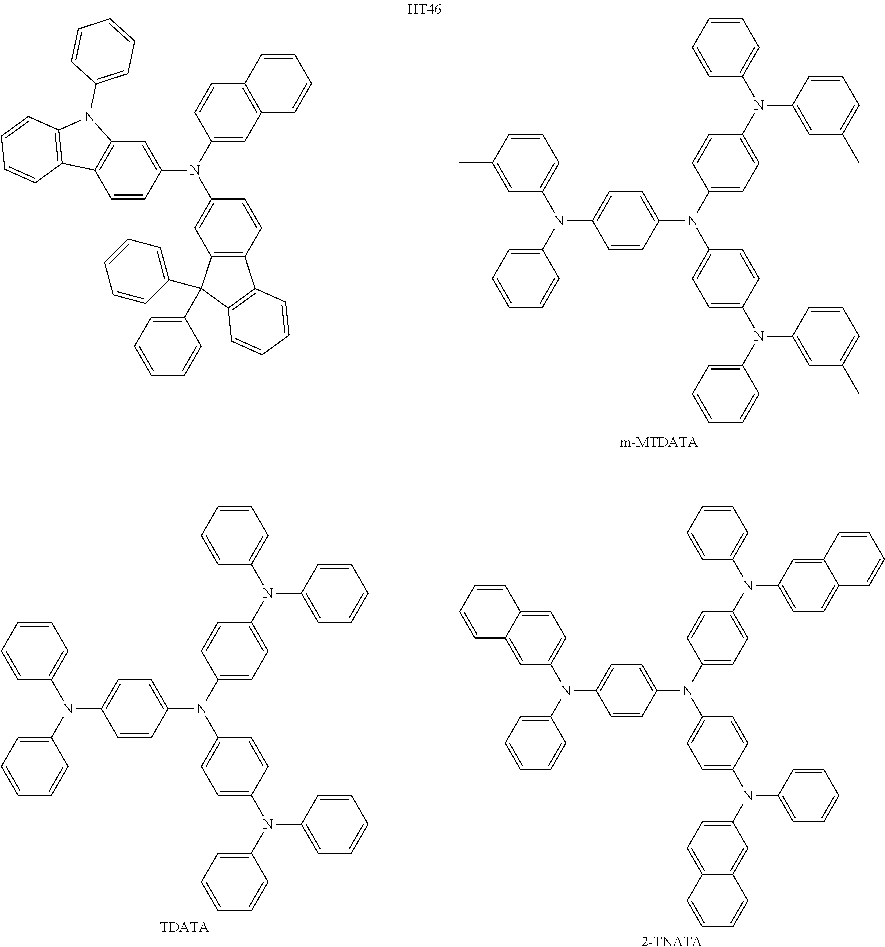

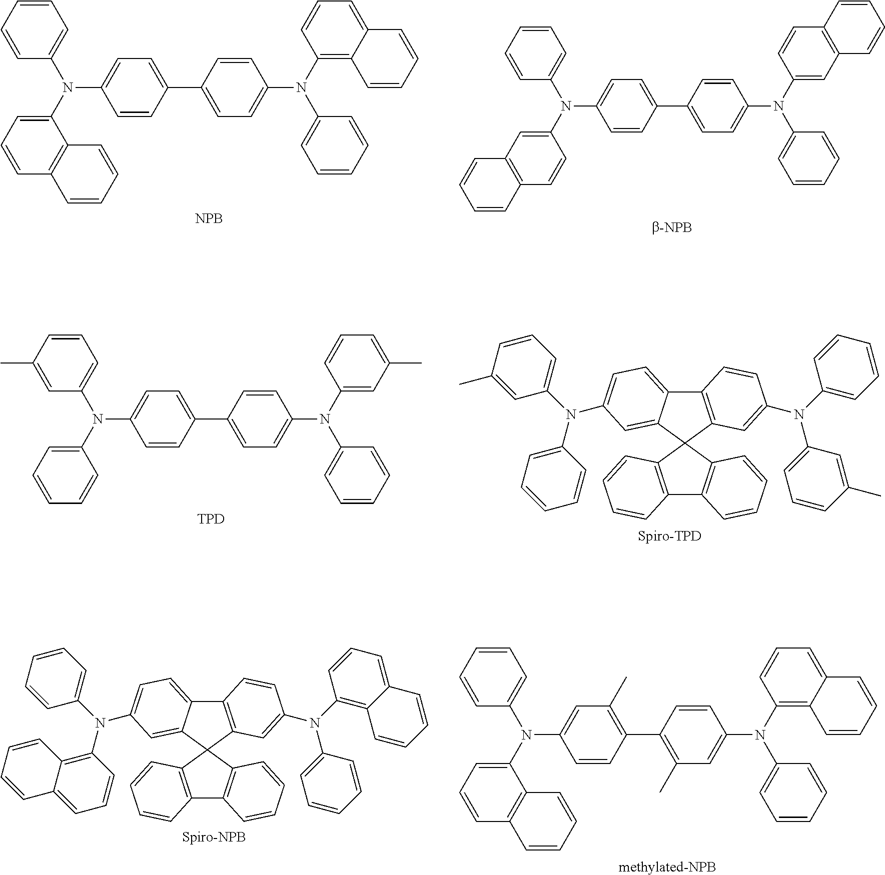

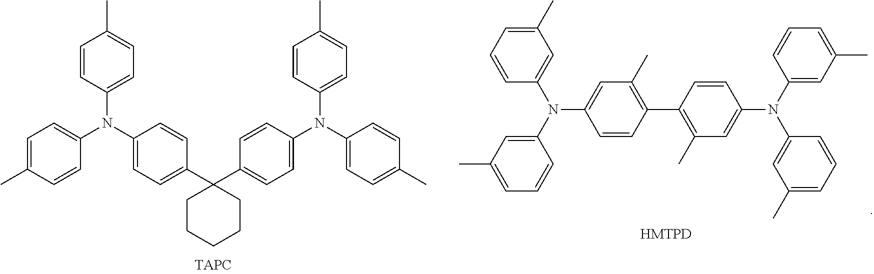

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, suitable or satisfactory hole transport characteristics may be obtained without a substantial increase in driving voltage.

The auxiliary layer may compensate for an optical resonance distance according to a wavelength of light introduced to the active layer 140 to increase light introduction efficiency. The electron-blocking layer may prevent or reduce leakage of electrons from the active layer 140 into the hole transport region. The hole transport material may be included in the auxiliary layer and the electron-blocking layer.

### Electron Transport Region

The charge auxiliary layers between the active layer 140 and the second electrode 170 may be collectively referred to as an electron transport region.

The electron transport region may have: i) a single-layered structure consisting of a single layer consisting of a single material, ii) a single-layered structure consisting of a single layer consisting of a plurality of different materials, or iii) a multi-layered structure including a plurality of layers including different materials.

The electron transport region may include a buffer layer, a hole-blocking layer, the electron transport layer 150, the electron injection layer 160, or any combination thereof.

For example, the electron transport region may have an electron transport layer/electron injection layer structure, a hole-blocking layer/electron transport layer/electron injection layer structure, or a buffer layer/electron transport layer/electron injection layer structure, the layers of each structure being sequentially stacked from the active layer 140.

The electron transport region (e.g., the buffer layer, the hole-blocking layer, or the electron transport layer in the electron transport region) may include a metal-free compound including at least one π electron-deficient nitrogen-containing C₁-C₆₀ cyclic group.

For example, the electron transport region may include a compound represented by Formula 601: Formula 601

[Ar₆₀₁]ₓₑ₁₁-[(L₆₀₁)ₓₑ₁-R₆₀₁]ₓₑ₂₁
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be as defined herein in connection with Q₁,
xe21 may be 1, 2, 3, 4, or 5, and
at least one selected from Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In one or more embodiments, Ar₆₀₁ in Formula 601 may be a substituted or unsubstituted anthracene group.

In one or more embodiments, the electron transport region may include a compound represented by Formula 601-1:

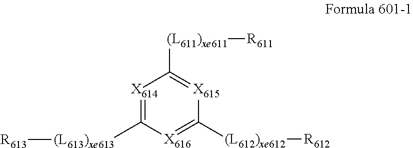

wherein, in Formula 601-1,
X₆₁₄ may be N or C(R₆₁₄), X₆₁₅ may be N or C(R₆₁₅), X₆₁₆ may be N or C(R₆₁₆), and at least one selected from X₆₁₄ to X₆₁₆ may be N,
L₆₁₁ to L₆₁₃ may each be as defined herein in connection with L₆₀₁,
xe611 to xe613 may each be as defined herein in connection with xe1,
R₆₁₁ to R₆₁₃ may each be as defined herein in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one selected from Compounds ET1 to ET45, 2,9-dimethyl-4,7-diphenyl-1,10-phenanthroline (BCP), 4,7-diphenyl-1,10-phenanthroline (Bphen), Alq3, BAlq, TAZ, NTAZ, or any combination thereof:

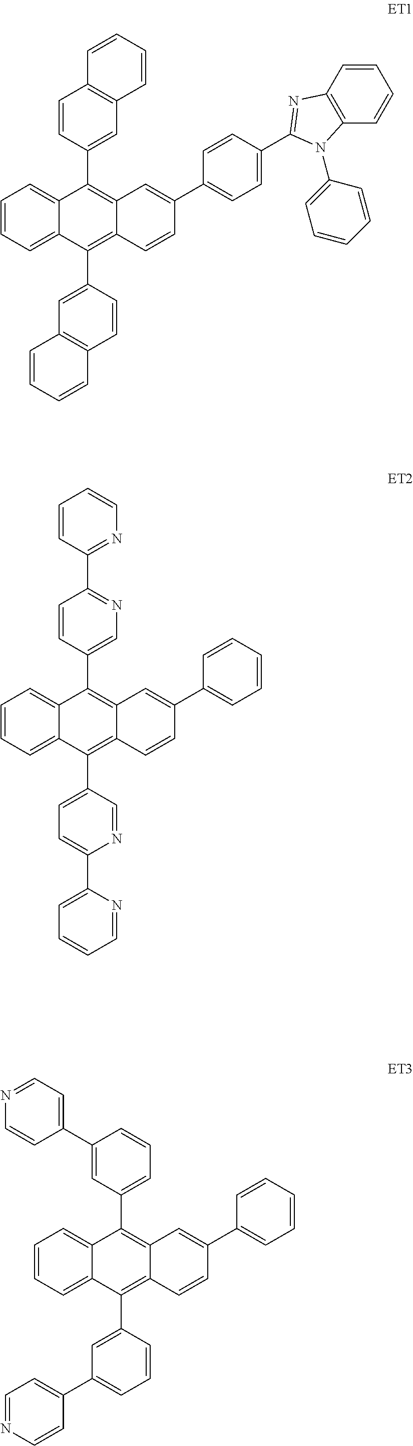 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

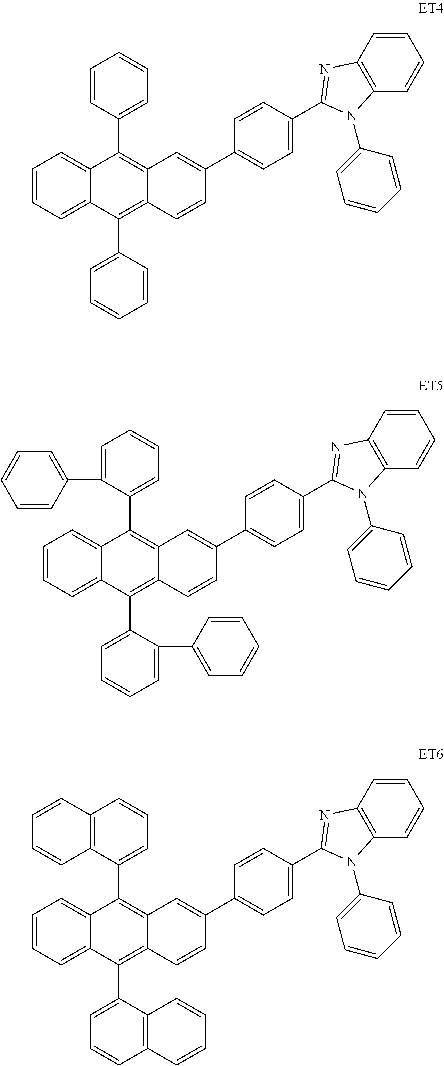 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

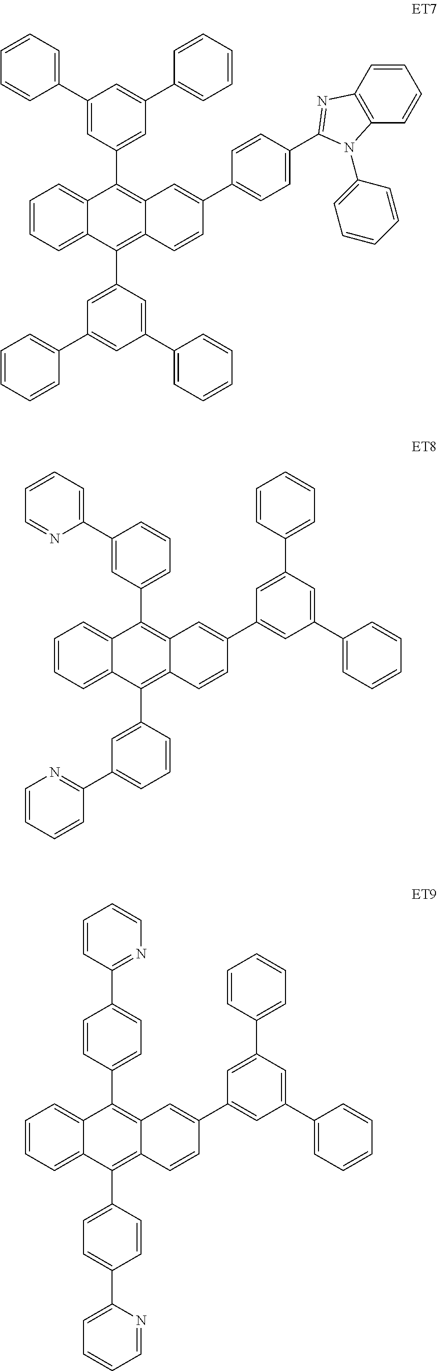 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

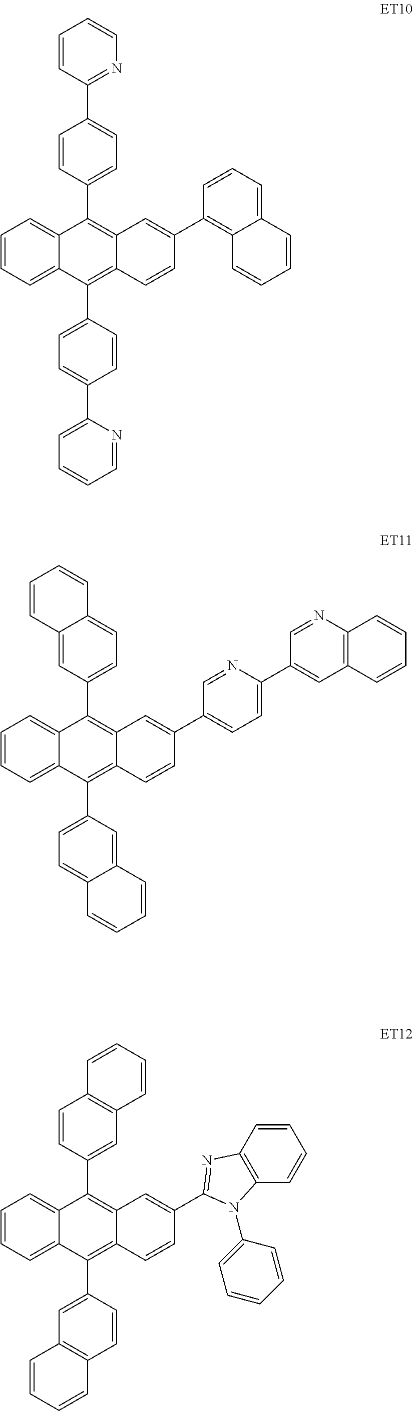 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

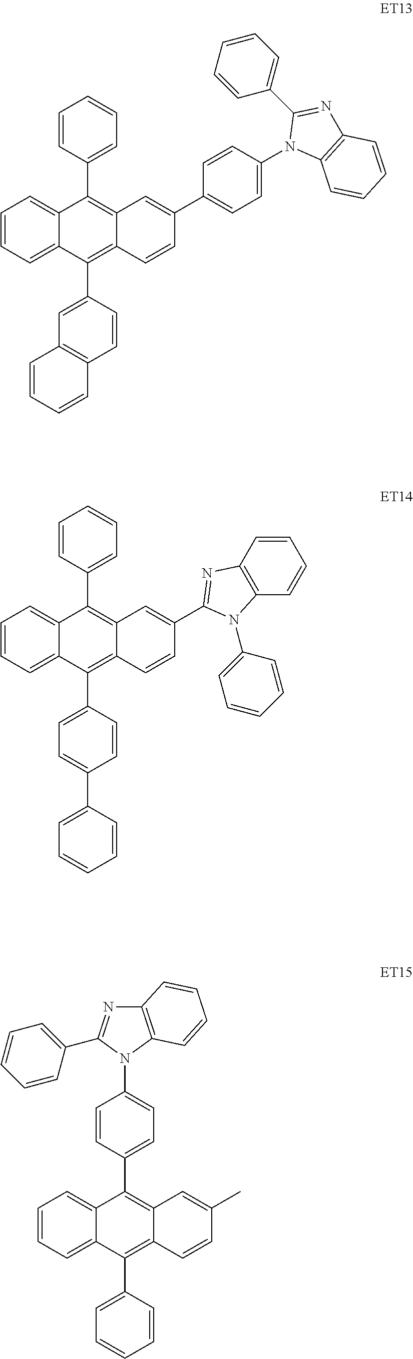 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

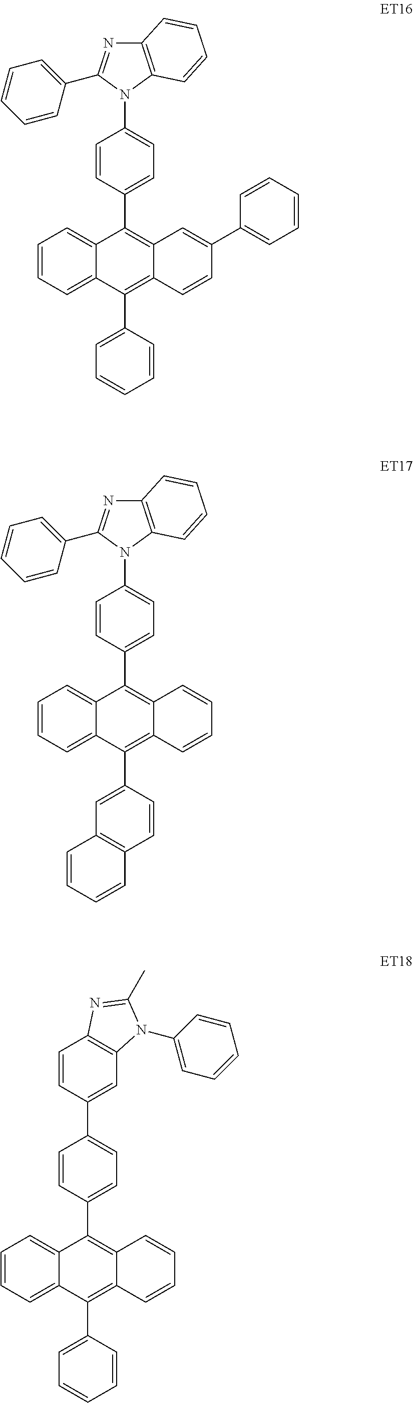 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

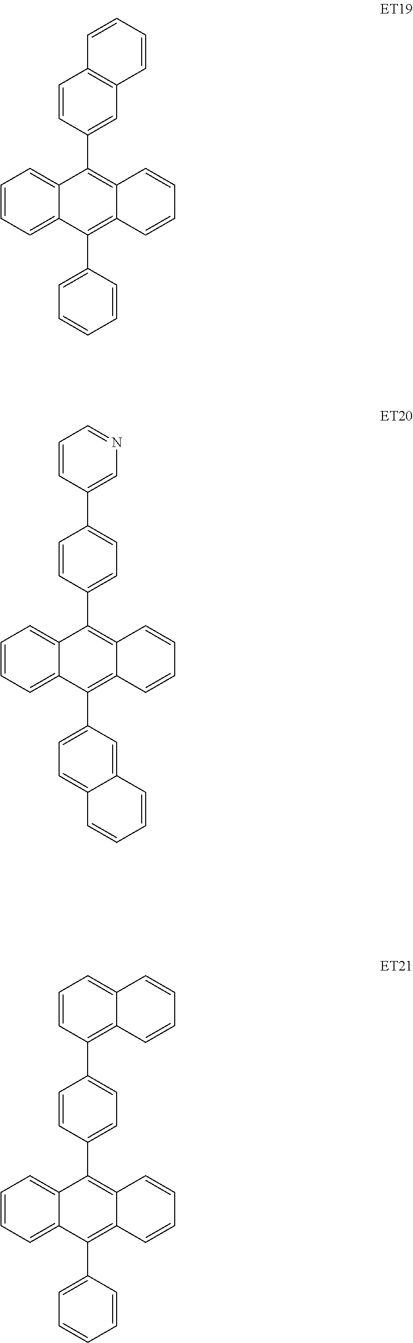

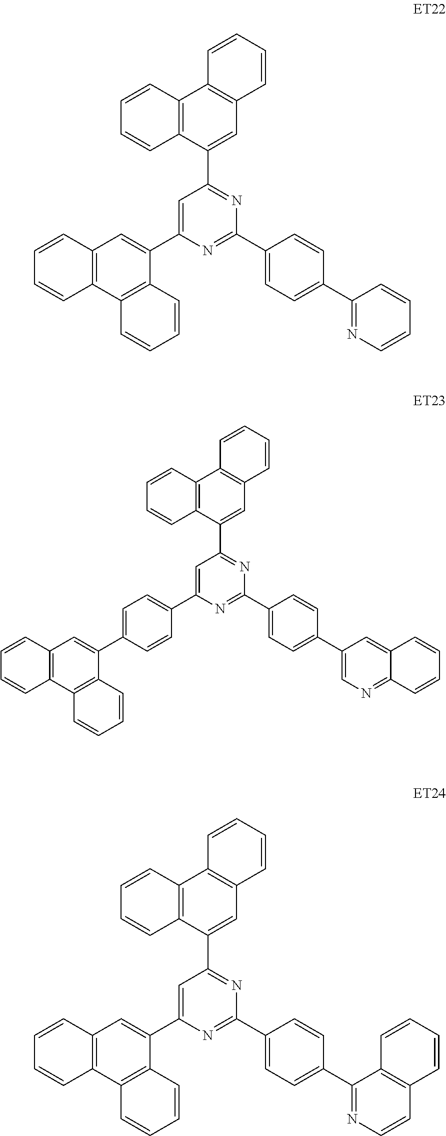 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

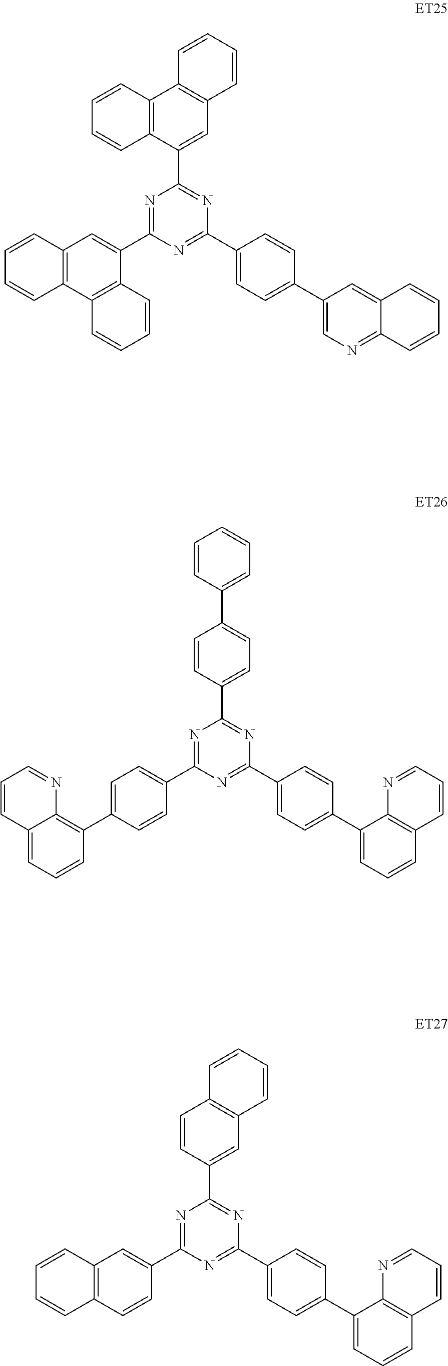 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

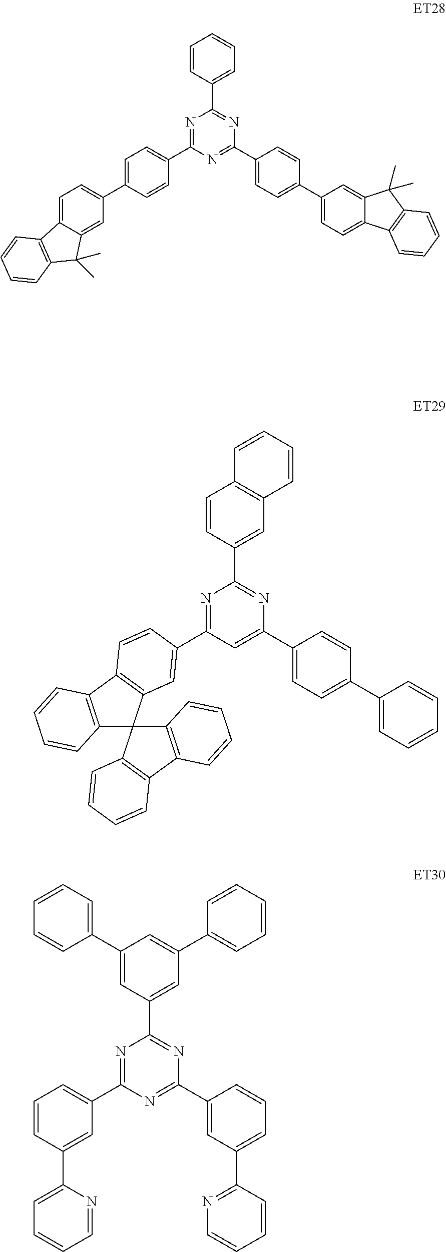 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

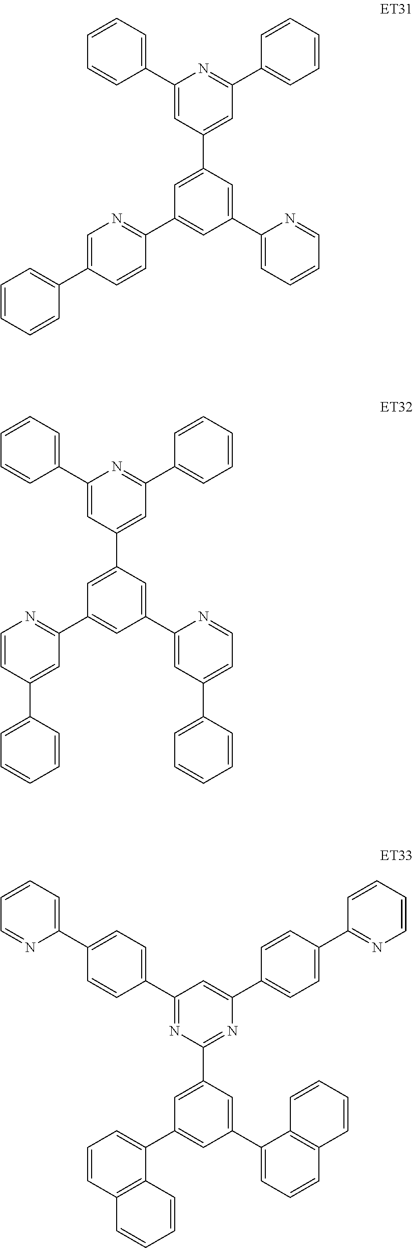 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

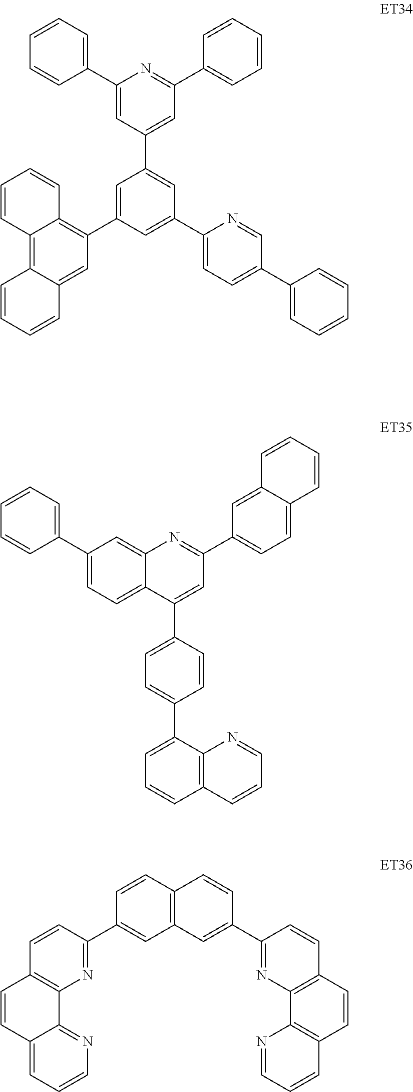 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

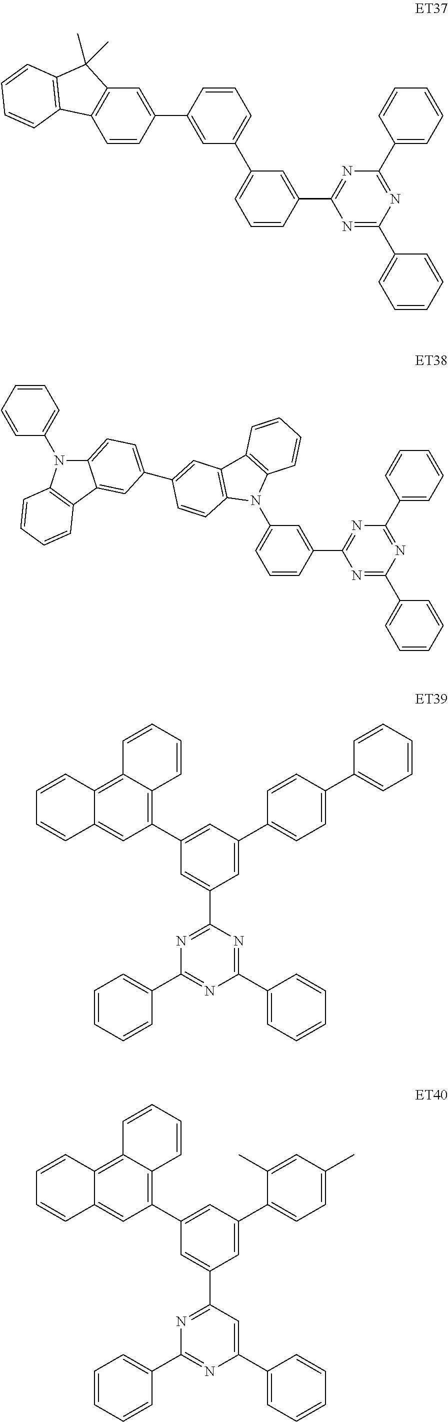 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

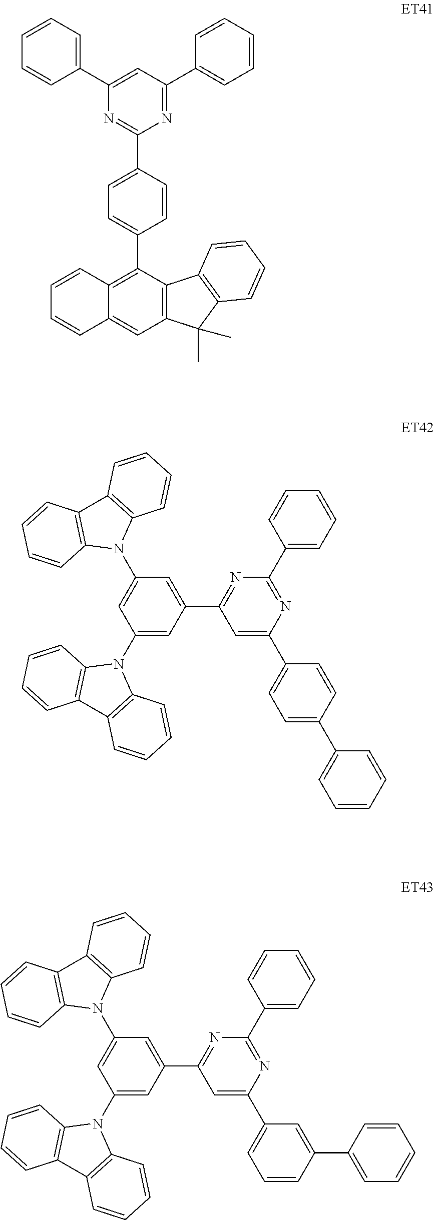 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

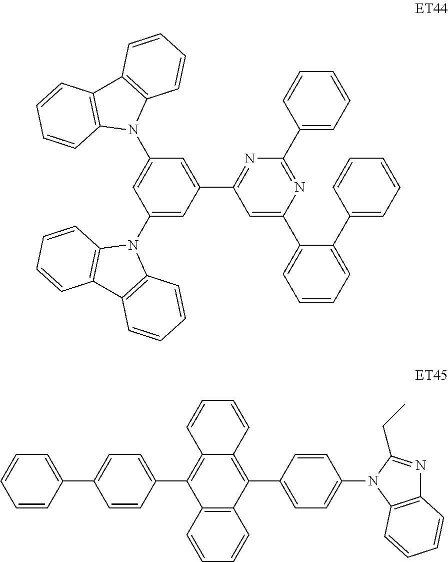 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

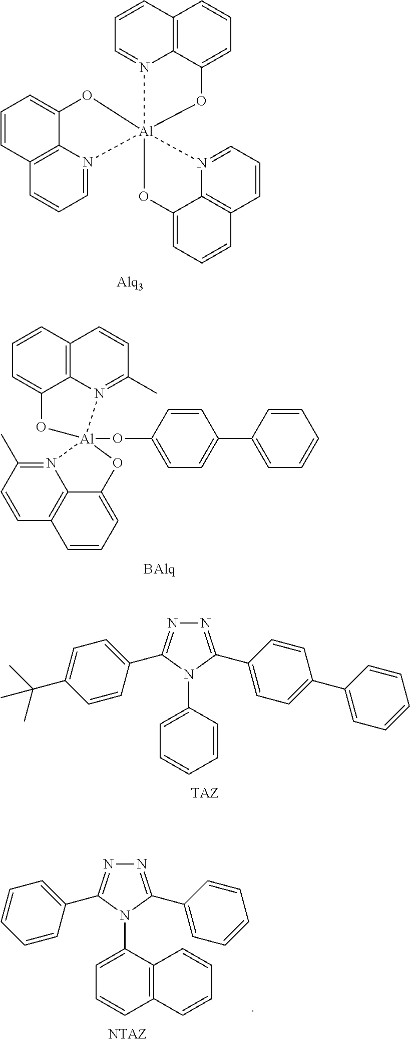

A thickness of the electron transport region may be in a range of about 50 Å to about 5,000 Å, for example, about 100 Å to about 4,000 Å. When the electron transport region includes a buffer layer, a hole-blocking layer, an electron transport layer, or any combination thereof, the buffer layer and the hole-blocking layer may each independently have a thickness in a range of about 20 Å to about 1,000 Å, for example, about 30 Å to about 300 Å, and the electron transport layer may have a thickness in a range of about 100 Å to about 1,000 Å, for example, about 150 Å to about 500 Å. When the thicknesses of the buffer layer, the hole-blocking layer, and/or the electron transport layer are within these ranges, suitable or satisfactory electron transport characteristics may be obtained without a substantial increase in driving voltage.

The electron transport region (e.g., the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) and/or ET-D2:

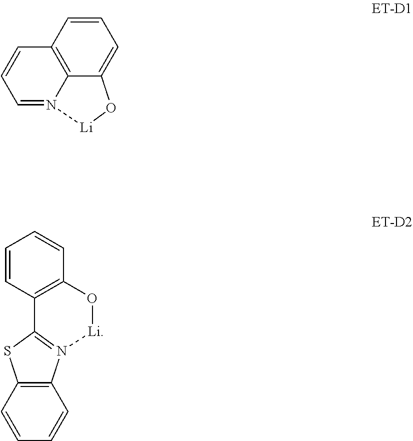

The electron transport region may include an electron injection layer that facilitates injection of electrons. The electron injection layer may be in direct contact with the second electrode 170.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (e.g., fluorides, chlorides, bromides, iodides, etc.), and/or tellurides of the alkali metal, the alkaline earth metal, and the rare earth metal, or any combination thereof.

The alkali metal-containing compound may include an alkali metal oxide, such as Li₂O, Cs₂O, and/or K₂O, an alkali metal halide, such as LiF, NaF, CsF, KF, LiI, NaI, CsI, and/or KI, or any combination thereof. The alkaline earth metal-containing compound may include an alkaline earth metal compound, such as BaO, SrO, CaO, BaₓSr₁₋ₓO (wherein x is a real number satisfying the condition of 0<x<1), and/or BaₓCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and the like.

The alkali metal complex, the alkaline earth-metal complex, and the rare earth metal complex may include i) one selected from ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In an embodiment, the electron injection layer may include (or consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., a compound represented by Formula 601).

In an embodiment, the electron injection layer may include (or consist of) i) an alkali metal-containing compound (e.g., an alkali metal halide), or ii) a) an alkali metal-containing compound (e.g., an alkali metal halide); and b) an alkali metal, an alkaline earth metal, a rare earth metal, or any combination thereof. For example, the electron injection layer may be a KI:Yb co-deposited layer, an RbI:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within these ranges, suitable or satisfactory electron injection characteristics may be obtained without a substantial increase in driving voltage.

### Second Electrode 170

The second electrode 170 may be over the active layer 140 or the electron transport region. The second electrode 170 may be a cathode, and as a material for forming the second electrode 170, a metal, an alloy, an electrically conductive compound, or any combination thereof, each having a low work function, may be used.

The second electrode 170 may include lithium (Li), silver (Ag), magnesium (Mg), aluminum (AI), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 170 may be a transmissive electrode, a semi-transmissive electrode, or a reflective electrode.

The second electrode 170 may have a single-layered structure or a multi-layered structure including a plurality of layers.

### Capping Layer

A first capping layer may be outside the first electrode 110, and/or a second capping layer may be outside the second electrode 170.

The first capping layer and/or second capping layer may prevent or reduce penetration of impurities, such as water and/or oxygen, into the organic photodetector 10, 20, or 30, thereby improving reliability of the organic photodetector 10, 20, or 30.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In an embodiment, at least one selected from the first capping layer and the second capping layer may each independently include an amine group-containing compound.

For example, at least one selected from the first capping layer and the second capping layer may each independently include a compound represented by Formula 201, a compound represented by Formula 202, or any combination thereof.

In one or more embodiments, at least one selected from the first capping layer and the second capping layer may each independently include one selected from Compounds HT28 to HT33, one selected from Compounds CP1 to CP6, β-NPB, or any combination thereof:

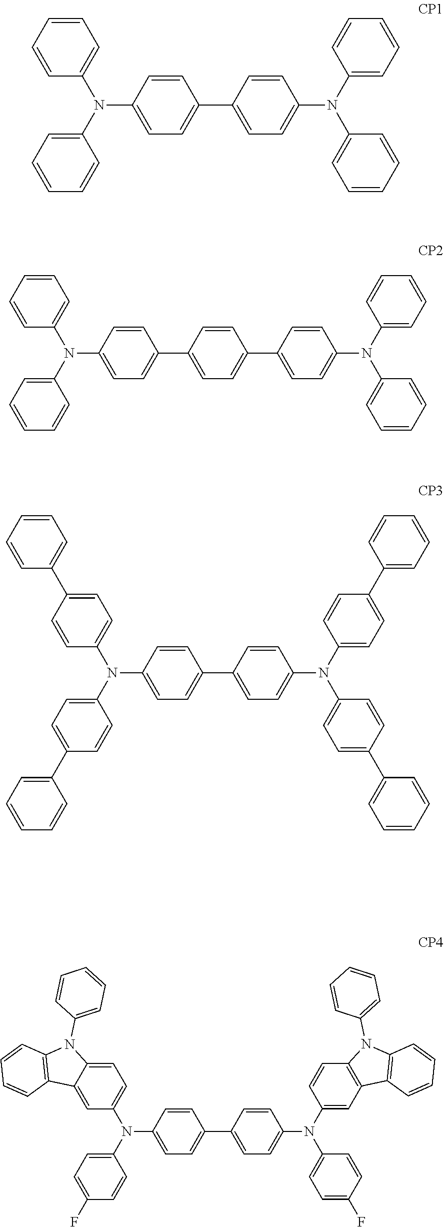 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

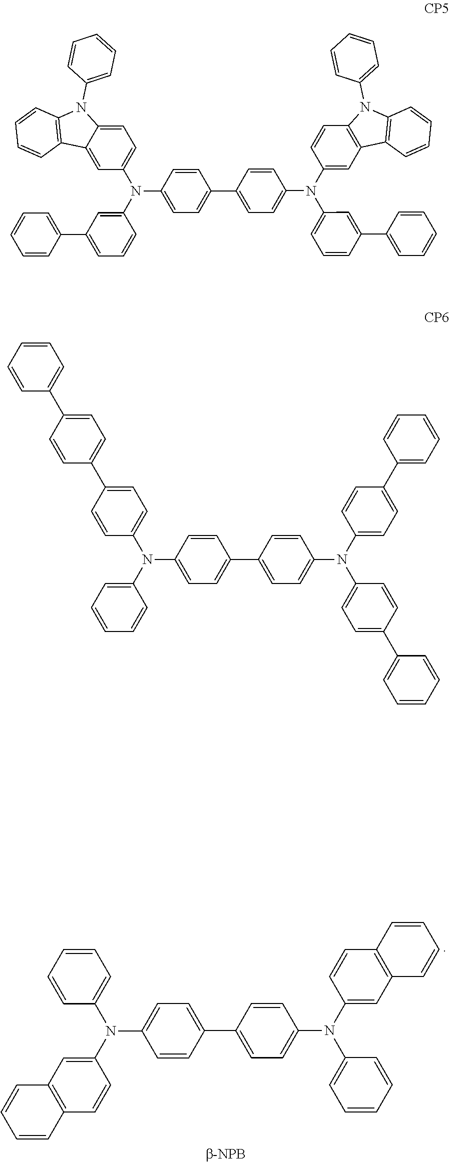 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

Provided is an electronic apparatus including the organic photodetector. For example, the electronic apparatus may further include a light-emitting device.

Accordingly, the electronic apparatus according to an embodiment may include: a substrate including a light detection region and an emission region;
an organic photodetector on the light detection region; and
a light-emitting device on the emission region,
wherein the organic photodetector may include: a first pixel electrode; a counter electrode facing the first pixel electrode; and a hole transport region, an active layer, and an electron transport region, which are sequentially between the first pixel electrode and the counter electrode,
the light-emitting device may include: a second pixel electrode; the counter electrode facing the second pixel electrode; and the hole transport region, an emission layer, and the electron transport region, which are sequentially between the second pixel electrode and the counter electrode,
the first pixel electrode and the active layer may correspond to the light detection region,
the second pixel electrode and the emission layer may correspond to the emission region,
the hole transport region, the electron transport region, and the counter electrode may be arranged throughout the light detection region and the emission region, and
the active layer may include a compound represented by Formula 1.
The hole transport region and the electron transport region may each be as described above.

For example, the hole injection layer, the hole transport layer, the electron transport layer, and/or the electron injection layer; and the counter electrode may be arranged throughout the light detection region and the emission region.

The electronic apparatus may be applied to various suitable displays, light sources, lighting, personal computers (e.g., a mobile personal computer), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (e.g., meters for a vehicle, an aircraft, and/or a vessel), projectors, and/or the like.

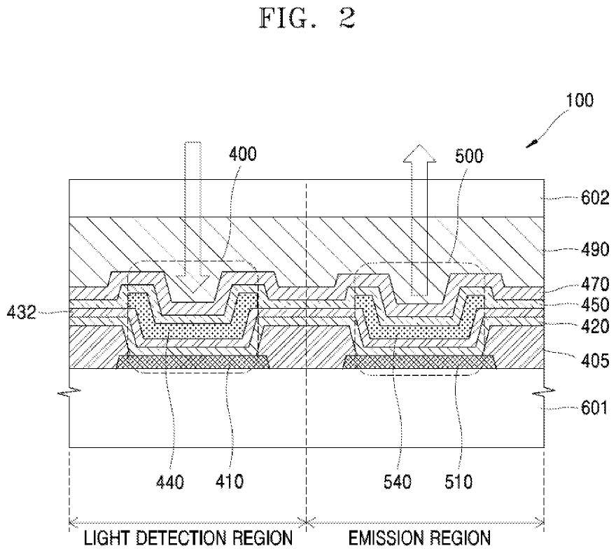

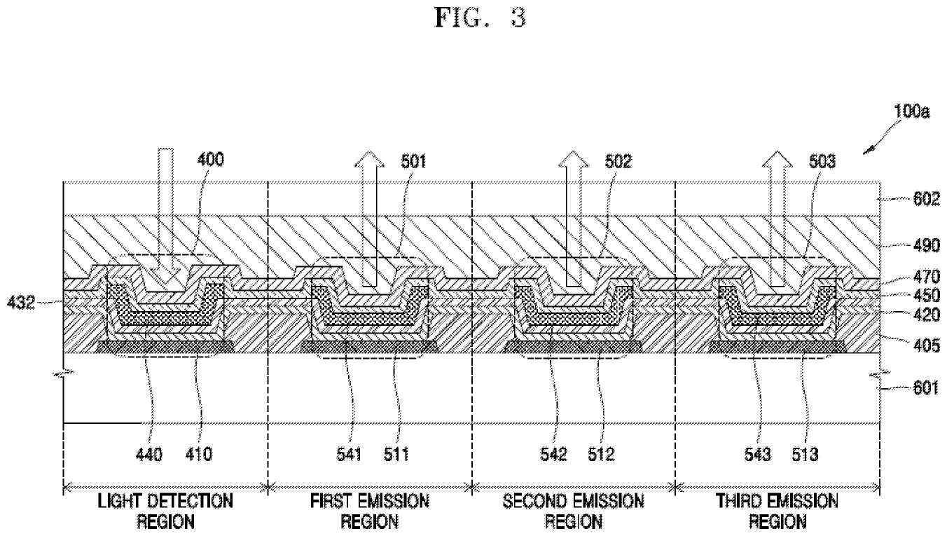

### Description of FIGS. 2-3

FIG. 2 is a schematic cross-sectional view of an electronic apparatus 100 according to an embodiment.

Referring to FIG. 2, the electronic apparatus 100 may include an organic photodetector 400 and a light-emitting device 500 between a substrate 601 and a substrate 602.

The substrate 601 and the substrate 602 may be a flexible substrate, a glass substrate, and/or a metal substrate. A buffer layer and a thin-film transistor may be on the substrate 601.

The buffer layer may prevent or reduce penetration of impurities through the substrate 601 and provide a flat surface on the substrate 601. The thin-film transistor may be on the buffer layer and may include an active layer, a gate electrode, a source electrode, and a drain electrode.

The thin-film transistor may be electrically connected to the light-emitting device 500 to drive the light-emitting device 500. One selected from the source electrode and drain electrode may be electrically connected to a second pixel electrode 510 of the light-emitting device 500.

Another thin-film transistor may be electrically connected to the organic photodetector 400. One selected from the source electrode and drain electrode may be electrically connected to a first pixel electrode 410 of the organic photodetector 400.

The organic photodetector 400 may include the first pixel electrode 410, a hole injection layer 420, a hole transport layer 432, an active layer 440, an electron transport layer 450, and a counter electrode 470.

In an embodiment, the first pixel electrode 410 may be an anode, and the counter electrode 470 may be a cathode. In one or more embodiments, as the organic photodetector 400 is driven by applying a reverse bias across the first pixel electrode 410 and the counter electrode 470, the electronic apparatus 100 may detect light incident onto the organic photodetector 400, generate charges, and extract the charges as a current.

The light-emitting device 500 may include the second pixel electrode 510, the hole injection layer 420, the hole transport layer 432, an emission layer 540, the electron transport layer 450, and the counter electrode 470.

In an embodiment, the second pixel electrode 510 may be an anode, and the counter electrode 470 may be a cathode. In one or more embodiments, in the light-emitting device 500, holes injected from the second pixel electrode 510 and electrons injected from the counter electrode 470 recombine in the emission layer 540 to generate excitons, which generate light by changing from an excited state to a ground state.

The first pixel electrode 410 and the second pixel electrode 510 may each be as described herein in connection with the first electrode 110.

A pixel-defining film 405 may be formed on an edge of the first pixel electrode 410 and on an edge of the second pixel electrode 510. The pixel-defining film 405 may define a pixel region and may electrically insulate between the first pixel electrode 410 and the second pixel electrode 510. The pixel-defining film 405 may include, for example, one or more various suitable organic insulating materials (e.g., a silicon-based material), inorganic insulating materials, or organic/inorganic composite insulating materials. The pixel-defining film 405 may be a transmissive film that transmits visible light or a blocking film that blocks (or reduces transmission of) visible light.

The hole injection layer 420 and the hole transport layer 432, which are common layers, are formed sequentially on the first pixel electrode 410 and the second pixel electrode 510. The hole injection layer 420 and the hole transport layer 432 may each be as described herein.

The active layer 440 is formed on the hole transport layer 432 to correspond to the light detection region. The active layer 440 may be the same as described herein.

The emission layer 540 is formed on the hole transport layer 432 to correspond to the emission region. In an embodiment, the light-emitting device 500 may further include, between the second pixel electrode 510 and the emission layer 540, an electron-blocking layer corresponding to the emission region.

As common layers for the entirety of the light detection region and the emission region, the electron transport layer 450 and the counter electrode 470 are sequentially formed on the active layer 440 and the emission layer 540. The electron transport layer 450 and the counter electrode 470 may be as described herein in connection with the electron transport layer and the second electrode 170, respectively.

The hole injection layer 420, the hole transport layer 432, and the electron transport layer 450 may each be arranged throughout the light detection region and the emission region.

As such, the manufacturing process of the electronic apparatus 100 may be simplified by arranging common layers for the organic photodetector 400 and the light-emitting device 500, existing functional layer materials used in the light-emitting device 500 may also be used for the organic photodetector 400, and thus, the organic photodetector 400 may be provided in-pixel in the electronic apparatus.

In an embodiment, an electron injection layer may be further included between the electron transport layer 450 and the counter electrode 470.

A capping layer may be on the counter electrode 470. A material for forming the capping layer may include the organic material and/or the inorganic material described herein. The capping layer may protect the organic photodetector 400 and the light-emitting device 500 and assist effective light emission from the light-emitting device 500.

An encapsulation portion 490 may be on the capping layer. The encapsulation portion 490 may be on the organic photodetector 400 and the light-emitting device 500 to protect the organic photodetector 400 and the light-emitting device 500 from water and/or oxygen. The encapsulation portion 490 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (e.g., polymethyl methacrylate, polyacrylic acid, etc.), an epoxy resin (e.g., aliphatic glycidyl ether (AGE), etc.), or any combination thereof; or a combination of the inorganic film and the organic film.

The electronic apparatus 100 may be, for example, a display apparatus. As the electronic apparatus 100 includes both the organic photodetector 400 and the light-emitting device 500, the electronic apparatus 100 may be a display apparatus having a light detection function.

In FIG. 2, the electronic apparatus 100 is illustrated as including one light-emitting device 500, but, as shown in FIG. 3, an electronic apparatus 100a according to an embodiment may include the organic photodetector 400, a first light-emitting device 501, a second light-emitting device 502, and a third light-emitting device 503.

Components illustrated in FIG. 3 may be understood by referring to the descriptions of the electronic apparatus 100.

The first light-emitting device 501 may include a second pixel electrode 511, the hole injection layer 420, the hole transport layer 432, a first emission layer 541, the electron transport layer 450, and the counter electrode 470.

The second light-emitting device 502 may include a third pixel electrode 512, the hole injection layer 420, the hole transport layer 432, a second emission layer 542, the electron transport layer 450, and the counter electrode 470.

The third light-emitting device 503 may include a fourth pixel electrode 513, the hole injection layer 420, the hole transport layer 432, a third emission layer 543, the electron transport layer 450, and the counter electrode 470.

The second pixel electrode 511, the third pixel electrode 512, and the fourth pixel electrode 513 may respectively correspond to a first emission region, a second emission region, and a third emission region, and may each be as described herein in connection with the first electrode 110.

The first emission layer 541 may correspond to the first emission region and emit a first color light, the second emission layer 542 may correspond to the second emission region and emit a second color light, and the third emission layer 543 may correspond to the third emission region and emit a third color light.

A maximum emission wavelength of the first color light, a maximum emission wavelength of the second color light, and a maximum emission wavelength of the third color light may be identical to or different from each other. For example, the maximum emission wavelength of the first color light and the maximum emission wavelength of the second color light may each be greater than the maximum emission wavelength of the third color light.

For example, the first color light may be red light, the second color light may be green light, and the third color light may be blue light, but embodiments are not limited thereto. Accordingly, the electronic apparatus 100a is capable of full-color emission. When a mixed light of the first color light, the second color light, and the third color light is white light, the first color light, the second color light, and the third color light may not be respectively limited to red light, green light, and blue light.

The organic photodetector 400, the first light-emitting device 501, the second light-emitting device 502, and the third light-emitting device 503 may be subpixels constituting a single pixel. In an embodiment, one pixel may include at least one organic photodetector 400.

The electronic apparatus 100a may be a display apparatus. As the electronic apparatus 100a includes all of the organic photodetector 400, the first light-emitting device 501, the second light-emitting device 502, and the third light-emitting device 503, the electronic apparatus 100a may be a full-color display apparatus having a light detection function.

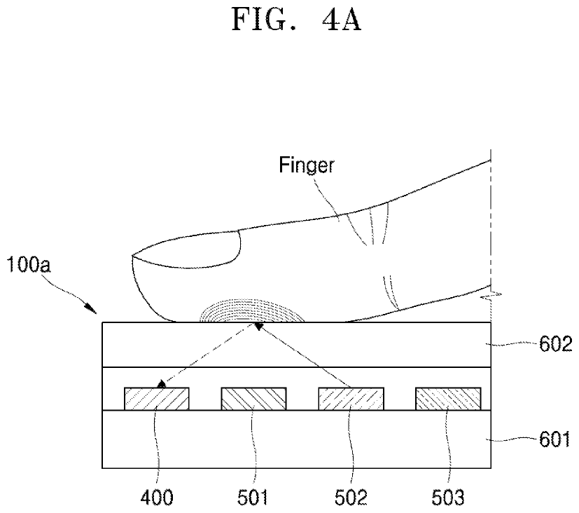

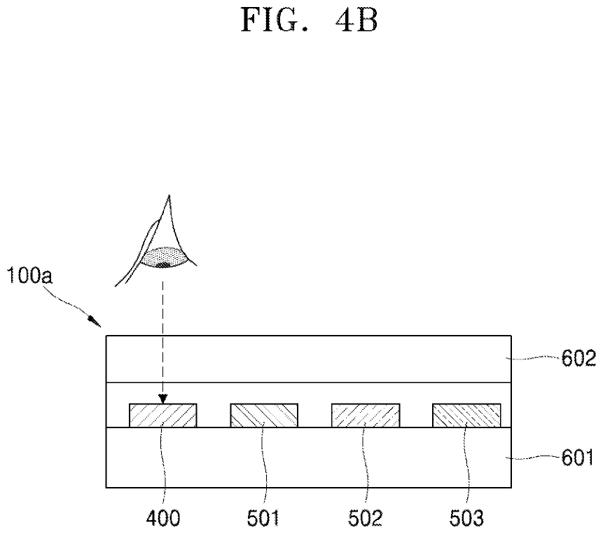

### Description of FIGS. 4A-4B

In the electronic apparatus 100a shown in FIG. 4A, the organic photodetector 400 and the light-emitting devices 501, 502, and 503 may be between the substrate 601 and the substrate 602.

For example, red light, green light, and blue light may be emitted from the light-emitting device 501, the light-emitting device 502, and the light-emitting device 503, respectively.

The electronic apparatus 100a according to an embodiment may have a function of detecting an object in contact with the electronic apparatus 100a, for example, a fingerprint of a finger. For example, as shown in FIG. 4A, at least some light emitted from the light-emitting device 502 and reflected by a fingerprint of a user may be re-incident on the organic photodetector 400, and thus, the organic photodetector 400 may detect the reflected light. A ridge in a fingerprint pattern of a finger may adhere to the substrate 602, and thus, the organic photodetector 400 may selectively obtain the fingerprint pattern of a user, for example, image information of the ridge. Although FIG. 4A shows an example in which information of an object in contact with the electronic apparatus 100a is obtained by using light emitted from the light-emitting device 502, light emitted from the light-emitting device 501 and/or light emitted from the light-emitting device 503 may also be used in substantially the same manner when obtaining information by using emitted light.

In an embodiment, as shown in FIG. 4B, the electronic apparatus 100a according to an embodiment may detect an object that is not in contact with the electronic apparatus 100a.

### Manufacturing Method

The layers constituting the hole transport region, the active layer, and the layers constituting the electron transport region may be formed in a set or specific region by using one or more suitable methods, such as vacuum deposition, spin coating, casting, Langmuir-Blodgett (LB) deposition, ink-jet printing, laser printing, and/or laser-induced thermal imaging.

When the layers constituting the hole transport region, the active layer, and the layers constituting the electron transport region are formed by vacuum deposition, the vacuum deposition may be performed at a deposition temperature in a range of about 100° C. to about 500° C., at a vacuum degree in a range of about 10⁻⁸ torr to about 10⁻³ torr, and at a deposition rate in a range of about 0.01 Å/sec to about 100 Å/sec, depending on the material to be included in each layer and the structure of each layer to be formed.

### DEFINITION OF TERMS

The term “C₃-C₆₀ carbocyclic group” as used herein refers to a cyclic group consisting of carbon atoms only as ring-forming atoms and having 3 to 60 carbon atoms, and the term “C₁-C₆₀ heterocyclic group” as used herein refers to a cyclic group that has 1 to 60 carbon atoms and further has, in addition to carbon atoms, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed together with each other. For example, the C₁-C₆₀ heterocyclic group may have 3 to 61 ring-forming atoms.

The term “cyclic group” as used herein may include both the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N═*′ as a ring-forming moiety, and the term “π electron-deficient nitrogen-containing C₁-C₆₀ cyclic group” as used herein refers to a heterocyclic group that has 1 to 60 carbon atoms and includes *—N═*′ as a ring-forming moiety.

For example,
the C₃-C₆₀ carbocyclic group may be i) a group T1 or ii) a condensed cyclic group in which two or more groups T1 are condensed together with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a group T2, ii) a condensed cyclic group in which two or more groups T2 are condensed together with each other, or iii) a condensed cyclic group in which at least one group T2 and at least one group T1 are condensed together with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) a group T1, ii) a condensed cyclic group in which two or more groups T1 are condensed together with each other, iii) a group T3, iv) a condensed cyclic group in which two or more groups T3 are condensed together with each other, or v) a condensed cyclic group in which at least one group T3 and at least one group T1 are condensed together with each other (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.), and
the π electron-deficient nitrogen-containing C₁-C₆₀ cyclic group may be i) a group T4, ii) a condensed cyclic group in which two or more groups T4 are condensed together with each other, iii) a condensed cyclic group in which at least one group T4 and at least one group T1 are condensed together with each other, iv) a condensed cyclic group in which at least one group T4 and at least one group T3 are condensed together with each other, or v) a condensed cyclic group in which at least one group T4, at least one group T1, and at least one group T3 are condensed together with each other (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
wherein the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the group T3 may be a furan group, a thiophene group, a 1H-pyrrole group, a silole group, or a borole group, and
the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” as used herein refers to a group condensed together with any suitable cyclic group, a monovalent group, or a polyvalent group (e.g., a divalent group, a trivalent group, a tetravalent group, etc.), depending on the structure of a formula in connection with which the terms are used. For example, the “benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and the like. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof may include an ethenyl group, a propenyl group, a butenyl group, and the like. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof may include an ethynyl group, a propynyl group, and the like. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof may include a methoxy group, an ethoxy group, an isopropyloxy group, and the like.

The term “C₃-C₁₀ cycloalkyl group” as used herein refers to a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantyl group, a norbornyl group (or a bicyclo[2.2.1]heptyl group), a bicyclo[1.1.1]pentyl group, a bicyclo[2.1.1]hexyl group, a bicyclo[2.2.2]octyl group, and the like. The term “C₃-C₁₀ cycloalkylene group” as used herein refers to a divalent group having substantially the same structure as the C₃-C₁₀ cycloalkyl group.

The term “C₁-C₁ heterocycloalkyl group” as used herein refers to a monovalent cyclic group that has 1 to 10 carbon atoms and further includes, in addition to carbon atoms, at least one heteroatom as a ring-forming atom, and examples thereof may include a 1,2,3,4-oxatriazolidinyl group, a tetrahydrofuranyl group, a tetrahydrothiophenyl group, and the like. The term “C₁-C₁ heterocycloalkylene group” as used herein refers to a divalent group having substantially the same structure as the C₁-C₁ heterocycloalkyl group.

The term “C₃-C₁₀ cycloalkenyl group” as used herein refers to a monovalent cyclic group that has 3 to 10 carbon atoms and at least one carbon-carbon double bond in the ring thereof and no aromaticity (e.g., is not aromatic), and examples thereof may include a cyclopentenyl group, a cyclohexenyl group, a cycloheptenyl group, and the like. The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group that has 1 to 10 carbon atoms, at least one heteroatom as a ring-forming atom, in addition to carbon atoms, and at least one double bond in the ring thereof. Examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and the like. The term “C₁-C₁₀ heterocycloalkenylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system of 6 to 60 carbon atoms, and the term “C₆-C₆₀ arylene group” as used herein refers to a divalent group having a carbocyclic aromatic system of 6 to 60 carbon atoms. Examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like. When the C₆-C₆₀ aryl group and the C₆-C₆₀ arylene group each include two or more rings, the two or more rings may be condensed together with each other.

The term “C₁-C₆₀ heteroaryl group” as used herein refers to a monovalent group having a heterocyclic aromatic system of 1 to 60 carbon atoms, further including, in addition to carbon atoms, at least one heteroatom as a ring-forming atom, and the term “C₁-C₆₀ heteroarylene group” as used herein refers to a divalent group having a heterocyclic aromatic system of 1 to 60 carbon atoms, further including, in addition to carbon atoms, at least one heteroatom as a ring-forming atom. Examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like. When the C₁-C₆₀ heteroaryl group and the C₁-C₆₀ heteroarylene group each include two or more rings, the two or more rings may be condensed together with each other.

The term “monovalent non-aromatic condensed polycyclic group” as used herein refers to a monovalent group having two or more rings condensed together with each other, only carbon atoms (e.g., having 8 to 60 carbon atoms) as ring-forming atoms, and non-aromaticity in its molecular structure when considered as a whole (e.g., is not aromatic when considered as a whole). Examples of the monovalent non-aromatic condensed polycyclic group may include an indenyl group, a fluorenyl group, a spiro-bifluorenyl group, a benzofluorenyl group, an indenophenanthrenyl group, an indenoanthracenyl group, and the like. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group having two or more rings condensed together with each other, at least one heteroatom, in addition to carbon atoms (e.g., having 1 to 60 carbon atoms), as a ring-forming atom, and non-aromaticity in its molecular structure when considered as a whole (e.g., is not aromatic when considered as a whole). Examples of the monovalent non-aromatic condensed heteropolycyclic group may include a 9,9-dihydroacridinyl group, a 9H-xanthenyl group, and the like. The term “divalent non-aromatic condensed heteropolycyclic group” as used herein refers to a divalent group having substantially the same structure as the monovalent non-aromatic condensed heteropolycyclic group.

The term “C₆-C₆₀ aryloxy group” as used herein refers to —OA₁₀₂ (wherein A₁₀₂ is the C₆-C₆₀ aryl group), and the term “C₆-C₆₀ arylthio group” as used herein refers to —SA₁₀₃ (wherein A₁₀₃ is the C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as used herein refers to any suitable atom other than a carbon atom. Examples of the heteroatom may include O, S, N, P, Si, B, Ge, Se, or any combination thereof.

The term “the third-row transition metal” as used herein includes hafnium (Hf), tantalum (Ta), tungsten (W), rhenium (Re), osmium (Os), iridium (Ir), platinum (Pt), gold (Au), and the like.

The term “Ph” as used herein refers to a phenyl group, the term “Me” as used herein refers to a methyl group, the term “Et” as used herein refers to an ethyl group, the term “ter-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group.” In other words, the “terphenyl group” is a substituted phenyl group having, as a substituent, a C₆-C₆₀ aryl group substituted with a C₆-C₆₀ aryl group.

### EXAMPLES

### Synthesis Examples

### Synthesis of Compound 132

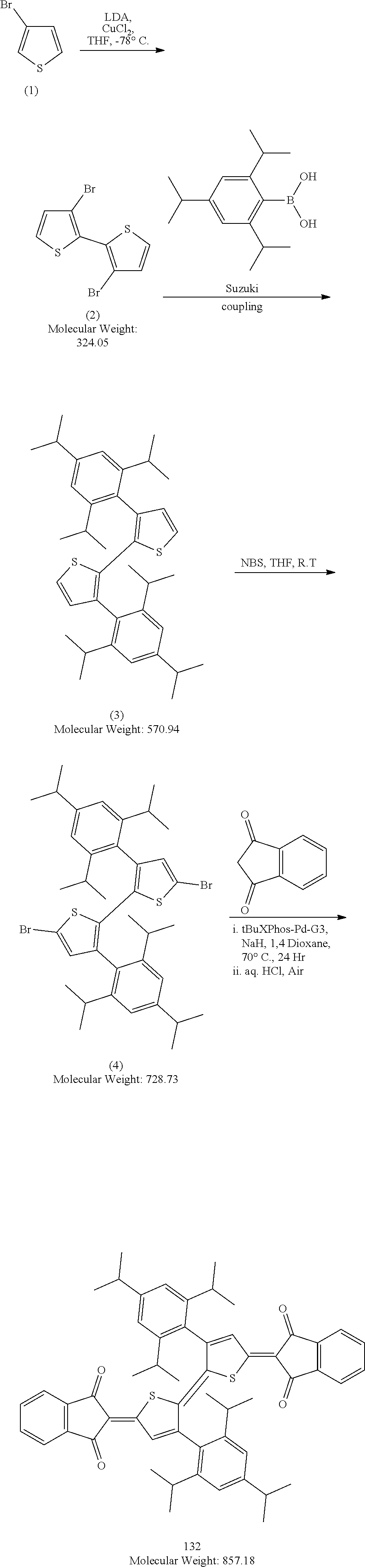

### Synthesis of Intermediate (2) (3,3′-dibromo-2,2′-bithiophene)

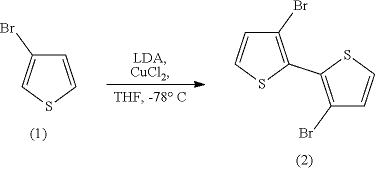 [SMILES: Brc1ccsc1 ; "LDA,CuCl2," ; Brc1ccsc1-c1sccc1Br]

In a round flask, Intermediate (1) (30 g, 181.01 mmol) dissolved in anhydrous tetrahydrofuran (THF) (150 mL) was stirred at −78° C. for 15 minutes, and then, lithium diisopropylamide (LDA) (184.01 mmol) was added dropwise thereto at the same temperature. After the addition of LDA, the resultant mixture was stirred for about 1 hour, and CuCl₂ was added thereto, followed by stirring for 1 hour. Then, the mixture was allowed for a reaction for 6 hours at room temperature. After completion of the reaction, the resultant was quenched using an aqueous ammonium chloride solution, and then, materials dissolved in organic solvents were extracted using dichloromethane (DCM) and H₂O. Moisture remaining after the extraction was removed by adding MgSO₄ thereto, the filtered solution was dissolved in DCM, and then, the solvent was removed therefrom using a rotary evaporator. Then, the resultant was separated by column chromatography to obtain Intermediate (2), which was a white solid. (Yield=19%)¹H NMR (500 MHz, CDCl₃) 7.44 (d, 2H), 7.12 (d, 2H).

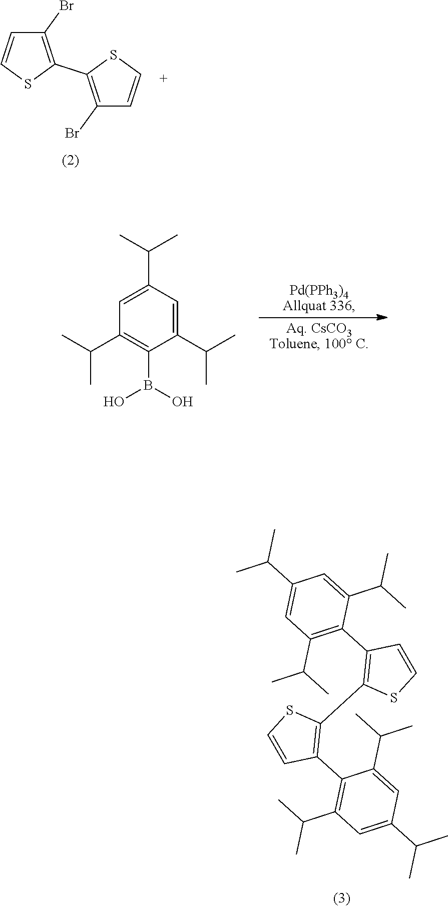 [SMILES: Brc1ccsc1-c1sccc1Br ; CC(C)c1cc(C(C)C)c(B(O)O)c(C(C)C)c1 ; "Aq. CsCO3Toluene, 100° C." ; "Pd(PPh3)4Allquat 336," ; CC(C)c1cc(C(C)C)c(-c2ccsc2-c2sccc2-c2c(C(C)C)cc(C(C)C)cc2C(C)C)c(C(C)C)c1]

### Synthesis of Intermediate (3) (3,3′-bis(2,4,6-triisopropylphenyl)-2,2′-bithiophene)

Intermediate (2) (200 mg, 0.61 mmol), 2,4,6-triisoproply phenyl boronic acid (612.6 mg, 2.46 mmol), and Aliquot 336 (3 drops) were added to a microwave vial, and then, Pd(PPh₃)₄ (35 mg, 0.03 mmol) was added thereto in a glove box. After anhydrous toluene (5 mL) and 2M CsCO₃ (3 mL) were added thereto and then dissolved, the resultant mixture was stirred at 100° C. for 12 hours. After completion of the reaction, the solvent was removed therefrom using a rotary evaporator, and an extraction process was performed thereon using diethyl ether and H₂O. After remaining moisture was removed using MgSO₄, the solvent of the filtered solution was removed using a rotary evaporator. Then, the resultant was separated by column chromatography to obtain Intermediate (3), which was a white solid. (Yield=45%)¹H NMR (500 MHz, CDCl₃) 7.12 (s, 4H), 7.02 (d, 2H), 6.73 (d, 2H), 3.04-2.99 (m, 2H), 2.68-2.62 (m, 4H), 1.38 (d, 12H), 1.10-1.05 (dd, 24H).

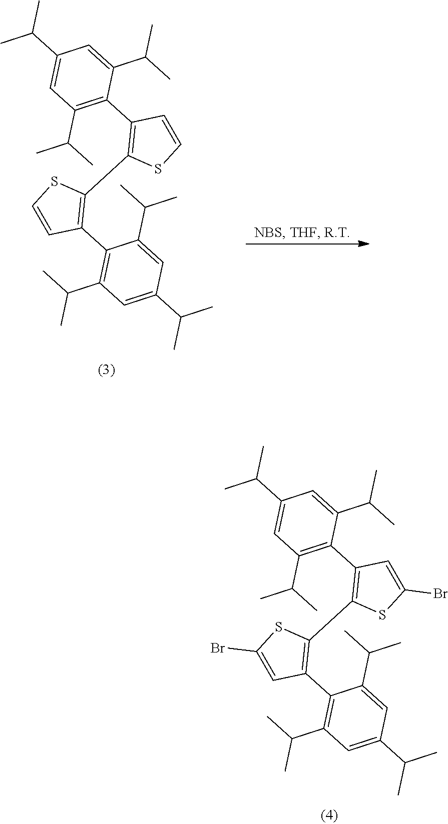 [SMILES: CC(C)c1cc(C(C)C)c(-c2ccsc2-c2sccc2-c2c(C(C)C)cc(C(C)C)cc2C(C)C)c(C(C)C)c1 ; "NBS, THF, R.T." ; CC(C)c1cc(C(C)C)c(-c2cc(Br)sc2-c2sc(Br)cc2-c2c(C(C)C)cc(C(C)C)cc2C(C)C)c(C(C)C)c1]

### Synthesis of Intermediate (4) (5,5′-dibromo-3,3′-bis(2,4,6-triisopropylphenyl)-2,2′-bithiophene)

Intermediate (3) (100 mg, 0.175 mmol) was dissolved in THF (150 mL), and then, the resultant mixture was stirred at room temperature for 10 minutes. N-Bromosuccinimide (NBS) (37.4 mg, 0.210 mmol) was added thereto, and the mixture was stirred for 6 hours under nitrogen conditions while light was blocked with aluminum foil. After completion of the reaction, the solvent was removed therefrom using a rotary evaporator, and then, materials dissolved in organic solvents were extracted using DCM and H₂O. Moisture remaining after the extraction was removed by adding MgSO₄ thereto, and then, the solvent was removed from the filtered solution by using a rotary evaporator. Then, the resultant was separated by column chromatography to obtain Intermediate (4), which was a light yellow solid. (Yield=95%)¹H NMR (500 MHz, CDCl₃) 7.13 (s, 4H), 6.67 (s, 2H), 3.06-2.98 (m, 2H), 2.67-2.60 (m, 4H), 1.37 (d, 12H), 1.13-1.07 (dd, 24H).

### Synthesis of Compound 132 (Q-IPBT-INDO, 2,2′-(3,3′-bis(2,4,6-triisopropylphenyl)-5H,5′H-[2,2′-bithiophenylidene]-5,5′-diylidene)bis(1H-indene-1,3(2H)-dione))

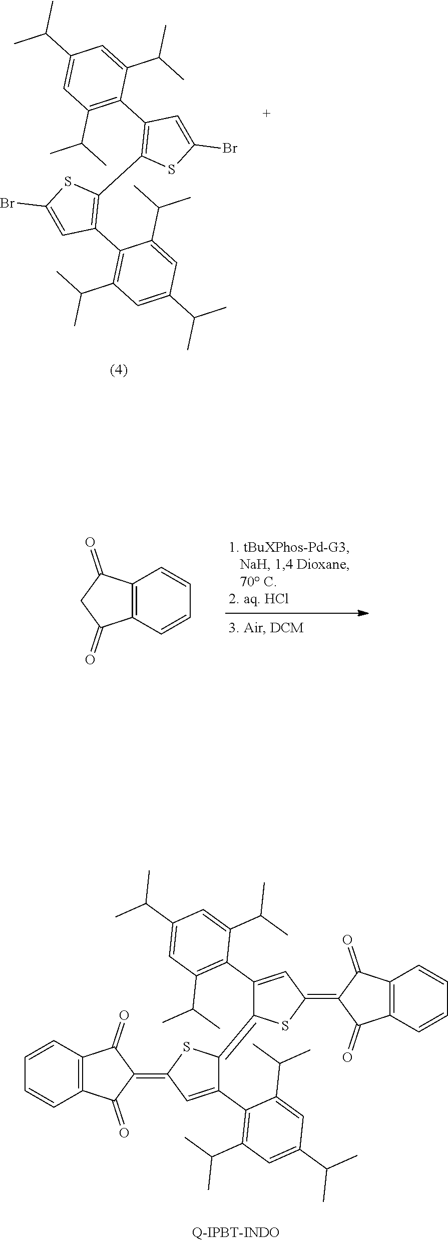 [SMILES: CC(C)c1cc(C(C)C)c(-c2cc(Br)sc2-c2sc(Br)cc2-c2c(C(C)C)cc(C(C)C)cc2C(C)C)c(C(C)C)c1 ; O=C1CC(=O)c2ccccc21 ; CC(C)c1cc(C(C)C)c(-c2cc(=C3C(=O)c4ccccc4C3=O)s/c2=c2/sc(=C3C(=O)c4ccccc4C3=O)cc2-c2c(C(C)C)cc(C(C)C)cc2C(C)C)c(C(C)C)c1]

After Intermediate (4), 1H-indene-1,3(2H)-dione, and NaH were added to a microwave vial, tBuXPhos Pd G3 (10.8 mg, 0.013 mmol) was added thereto in a glove box, which was then sealed. 1,4-dioxane (5 mL) was added thereto, and then, the resultant mixture was heated to 70° C. and stirred while light was blocked with aluminum foil. After completion of the reaction, the solvent was removed therefrom, and 15 mL of 0.1 M HCl was added thereto. The filtered material was dissolved in DCM, and the resultant mixture was stirred in air. The mixture was sufficiently exposed to room temperature and then subjected to thin-layer chromatography (TLC) to confirm synthesis of the quinoid material. Then, the mixture was separated by column chromatography using DCM:hexane (1:1) as eluents to obtain Q-IPBT-INDO, which was a blue solid. (Yield=40%)¹H NMR (500 MHz, CDCl₃) 8.46 (s, 2H), 7.84-7.79 (m, 4H), 7.68-7.64 (m, 4H), 7.27 (s, 4H), 3.16-3.11 (m, 2H), 2.59-2.52 (m, 4H), 1.50 (d, 12H), 1.22-1.14 (dd, 24H).

### Synthesis of Compound 140

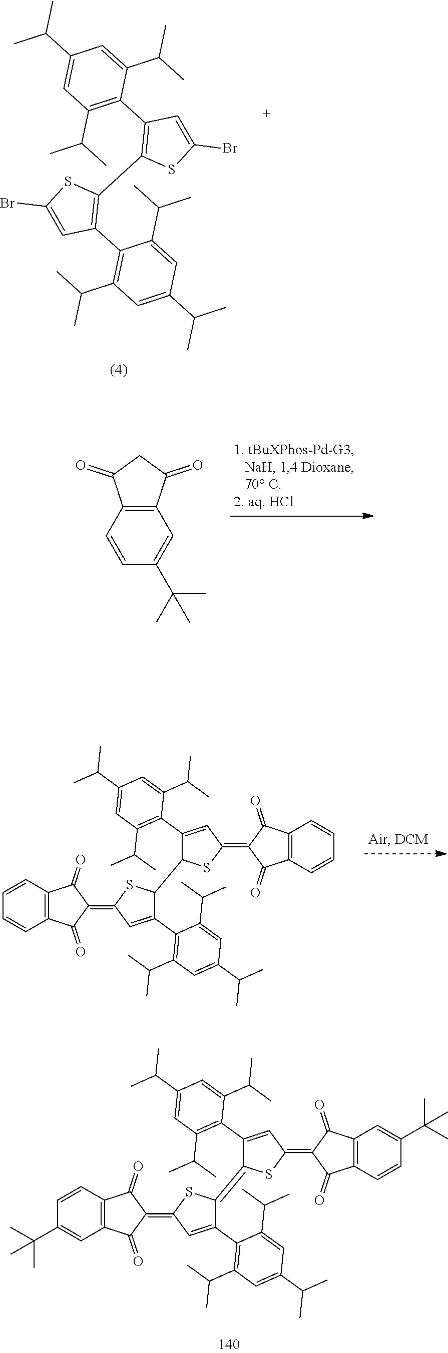 [SMILES: CC(C)c1cc(C(C)C)c(-c2cc(Br)sc2-c2sc(Br)cc2-c2c(C(C)C)cc(C(C)C)cc2C(C)C)c(C(C)C)c1 ; CC(C)(C)c1ccc2c(c1)C(=O)CC2=O ; CC(C)c1cc(C(C)C)c(C2=CC(=C3C(=O)c4ccccc4C3=O)SC2C2SC(=C3C(=O)c4ccccc4C3=O)C=C2c2c(C(C)C)cc(C(C)C)cc2C(C)C)c(C(C)C)c1 ; CC(C)c1cc(C(C)C)c(-c2c/c(=C3/C(=O)c4ccc(C(C)(C)C)cc4C3=O)s/c2=c2/s/c(=C3\C(=O)c4ccc(C(C)(C)C)cc4C3=O)cc2-c2c(C(C)C)cc(C(C)C)cc2C(C)C)c(C(C)C)c1]

Compound 140 was synthesized in substantially the same manner as used to synthesize Compound 132, except that 5-tert-butyl-1H-indene-1,3(2H)-dione was used instead of 1H-indene-1,3(2H)-dione in Synthesis of Compound 132. Compound 140 thus produced was identified by ¹H NMR. (Blue solid, Yield=38%)¹H NMR (500 MHz, CDCl₃) 8.45 (s, 2H), 7.78 (d, 2H), 7.71-7.70 (m, 4H), 7.27 (s, 4H), 3.16-3.12 (m, 2H), 2.60-2.54 (m, 4H), 1.49 (t, 12H), 1.36-1.35 (d, 18H), 1.22-1.14 (dd, 24H).

### Synthesis of Compound 141

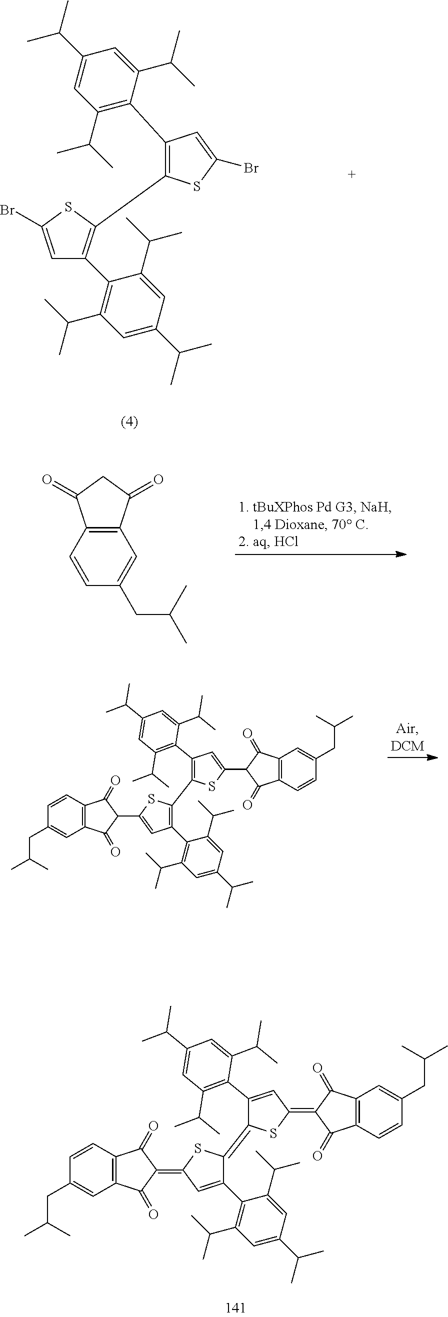

Compound 141 was synthesized in substantially the same manner as used to synthesize Compound 132, except that 5-isobutyl-1H-indene-1,3(2H)-dione was used instead of 1H-indene-1,3(2H)-dione in Synthesis of Compound 132. Compound 141 thus produced was identified by ¹H NMR. (Blue solid, Yield=38%) ¹H NMR (500 MHz, CDCl₃) 8.45 (s, 2H), 7.72 (d, 4H), 7.60-7.52 (m, 4H), 7.27 (s, 4H), 3.16-3.12 (m, 2H), 2.60-2.54 (m, 8H), 1.49 (t, 12H), 1.21-1.35 (dd, 12H), 1.22-1.14 (dd, 24H).

### Synthesis of Compound 168

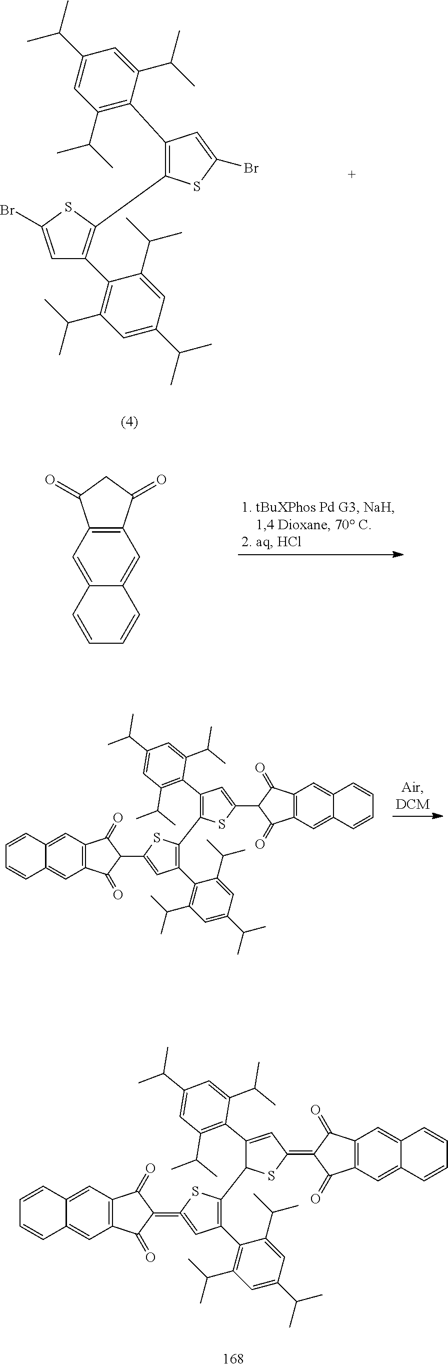

Compound 168 was synthesized in substantially the same manner as used to synthesize Compound 132, except that 1H-cyclopenta[b]naphthalene-1,3(2H)-dione was used instead of 1H-indene-1,3(2H)-dione in Synthesis of Compound 132. Compound 168 thus produced was identified by ¹H NMR. (Blue solid, Yield=38%) ¹H NMR (500 MHz, CDCl₃) 8.59 (s, 2H), 8.33-8.28 (d, 4H), 8.02-8.01 (m, 4H), 7.64-7.63 (m, 4H), 7.31 (s, 4H) 3.20-3.14 (m, 2H), 2.62-2.56 (m, 4H), 1.53 (d, 12H), 1.22-1.17 (dd, 24H).

### Synthesis of Compounds 143 and 169

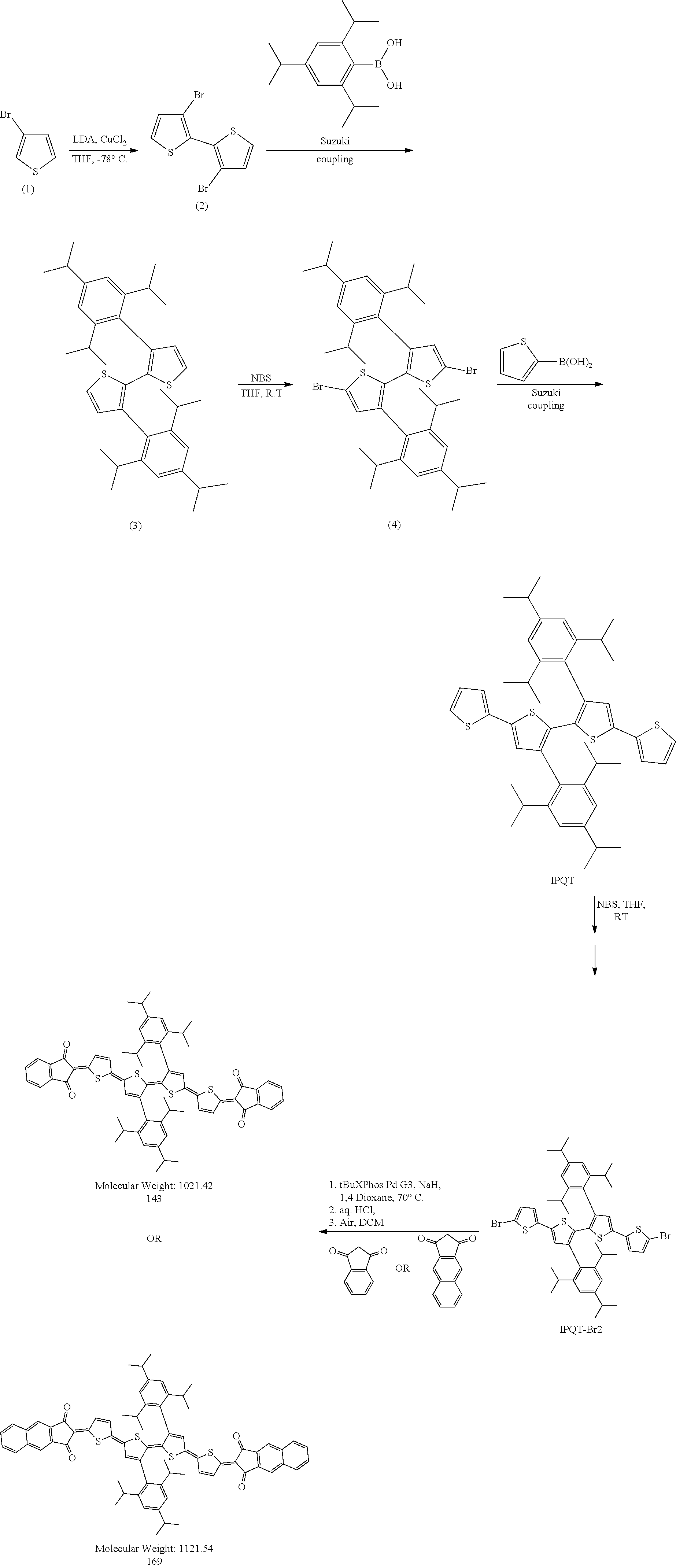

### Synthesis of Intermediate IPQT (3″, 4′-bis (2, 4, 6-triisopropylphenyl)-2, 2′:5′, 2″:5″, 2″′-quaterthiophene)

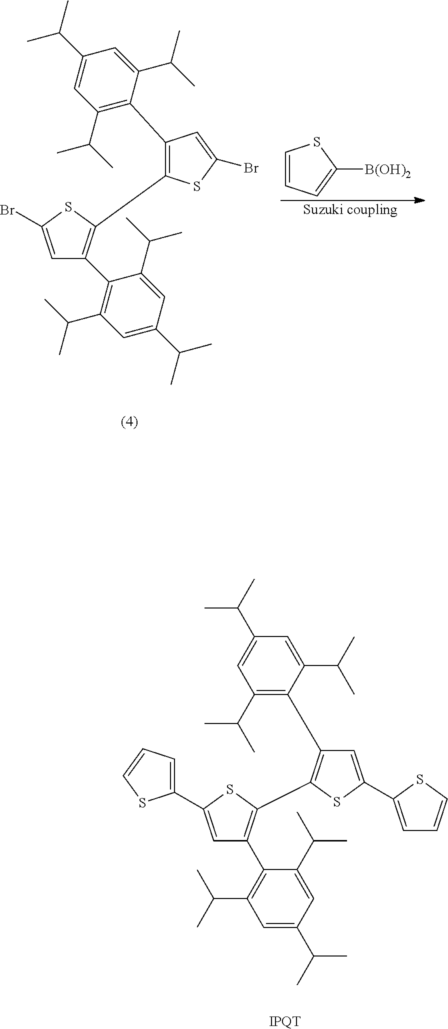

After Intermediate (4) and thiophen-2-yl boronic acid (131.6 mg, 12.02 mmol) added to a microwave vial, Pd(dppf)₂Cl₂ (29.9 mg, 0.04 mmol) was added thereto in a glove box, which was then sealed. Anhydrous toluene (4 mL), anhydrous THF (2 mL), and 2M Na₂CO₃ (3 mL) were added thereto, and then, the resultant mixture was stirred at 100° for 12 hours. After synthesis of the material was confirmed by TLC, the solvent of the mixture was removed using a rotary evaporator. Then, an extraction process was performed thereon using diethyl ether and H₂O, and MgSO₄ was added thereto to remove moisture therefrom. After the solvent was removed therefrom using a rotary evaporator, column chromatography was performed thereon using hexane to obtain IPQT, which was a yellow solid. (Yield=59%) ¹H NMR (500 MHz, CDCl₃) 7.15 (s, 4H), 7.11 (d, 2H), 6.93 (t, 2H), 6.87 (d, 2H), 6.85 (s, 2H), 3.04-2.99 (m, 2H), 2.75-2.70 (m, 4H), 1.38 (d, 12H), 1.11-1.05 (dd, 24H).

### Synthesis of Compound IPQT-Br2 (5,5″′-dibromo-3″,4′-bis(2,4,6-triisopropylphenyl)-2,2′:5′,2″:5″,2″′-quaterthiophene)

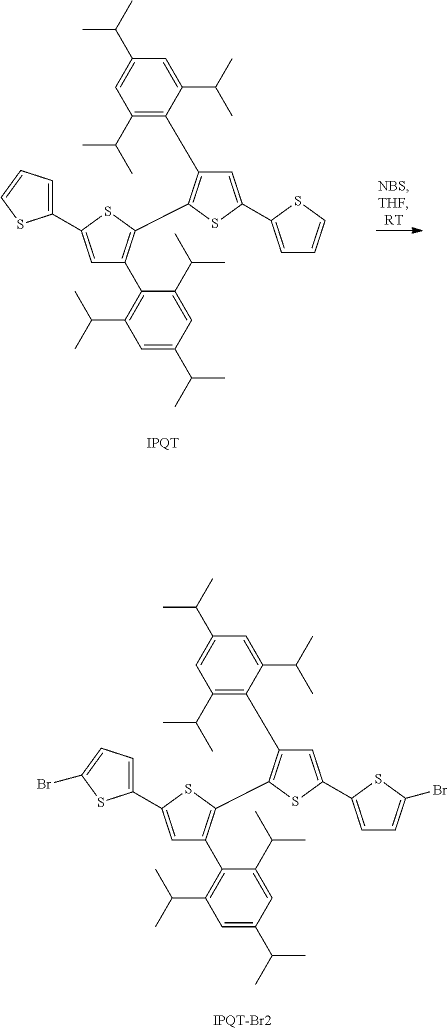

Under nitrogen conditions, Intermediate IPQT (200 mg, 0.27 mmol) was added to anhydrous THF (250 mL), and the resultant mixture was stirred at 0° C. for 10 minutes. Then, NBS (96.3 mg, 0.54 mmol) was added thereto while light was blocked by aluminum foil, and the mixture was stirred at the same temperature for 15 minutes. After completion of the reaction, the solvent was removed therefrom using a rotary evaporator, an extraction process was performed thereon using DCM and H₂O, and remaining water was removed therefrom using MgSO₄. After the solvent was removed therefrom using a rotary evaporator, the obtained organic layer was subjected to column chromatography using hexane to obtain IPQT-Br2, which was a light yellow solid. (Yield=62%)¹H NMR (500 MHz, CDCl₃) 7.13 (s, 4H), 6.87 (d, 2H), 6.77 (s, 2H), 6.60 (d, 2H), 3.04-2.98 (m, 2H), 2.69-2.64 (m, 4H), 1.37 (d, 12H), 1.10-1.06 (dd, 24H).

### Synthesis of Compound 143 (Q-IPQT-INDO, 2,2′-((2,2″,2″′)-3″,4′-bis(2,4,6-triisopropylphenyl)-5H,5″′H-[2,2′:5′,2″:5″,2″′-quaterthiophene]-5,5″′-diylidene)bis(1H-indene-1,3(2H)-dione))

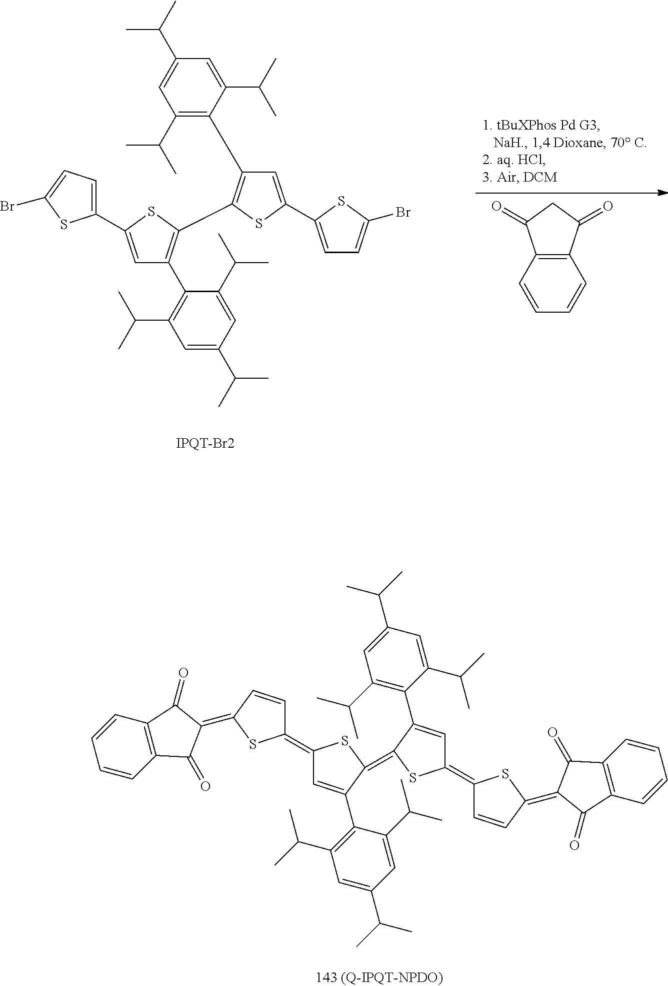

Compound 143 was synthesized in substantially the same manner as used to synthesize Compound 132, except that IPQT-Br2 was used instead of Intermediate (4) in Synthesis of Compound 132. Compound 143 thus produced was identified by ¹H NMR (CDCl₃, 500 MHz). (Blue solid, Yield=34%)¹H NMR (500 MHz, CDCl₃) 8.50 (m, 4H), 8.46 (s, 2H), 7.84-7.79 (m, 4H), 7.68-7.64 (m, 4H), 7.27 (s, 4H), 3.16-3.11 (m, 2H), 2.59-2.52 (m, 4H), 1.50 (d, 12H), 1.22-1.14 (dd, 24H).

### Synthesis of Compound 169 (Q-IPQT-NPDO, 2,2′-((2,2″,2″′)-3″,4′-bis(2,4,6-triisopropylphenyl)-5H,5″′H-[2,2′:5′,2″:5″,2″′-quaterthiophene]-5,5″′-diylidene)bis(1H-cyclopenta[b]naphthalene-1,3(2H)-dione))

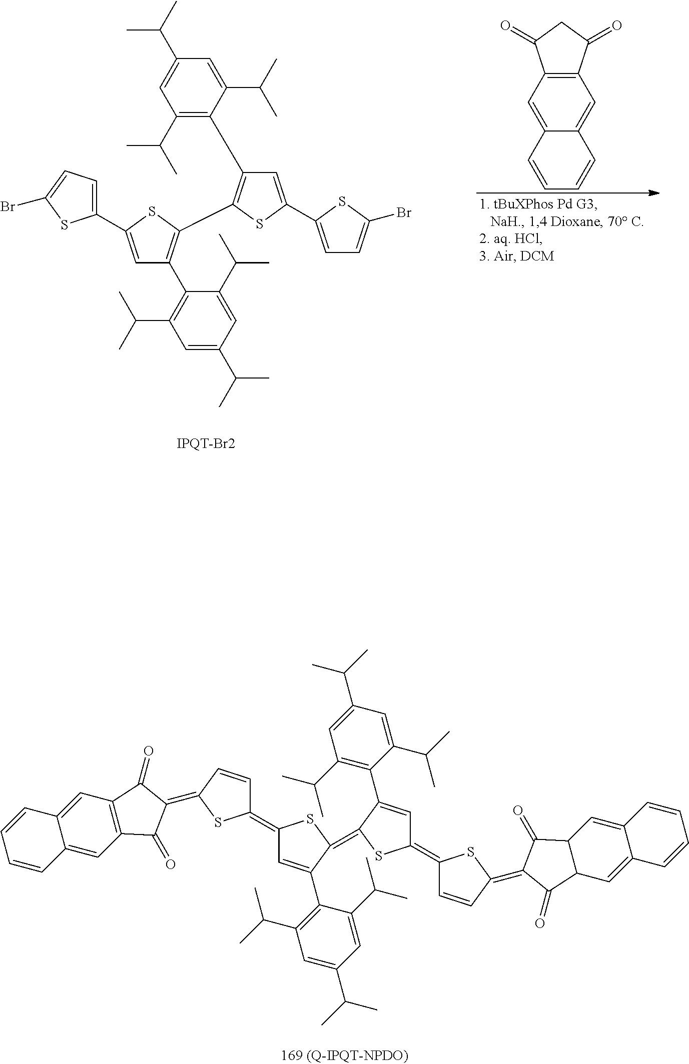

Compound 169 was synthesized in substantially the same manner as used to synthesize Compound 132, except that, in Synthesis of Compound 132, IPQT-Br2 was used instead of Intermediate (4), and 1H-cyclopenta[b]naphthalene-1,3(2H)-dione was used instead of 1H-indene-1,3(2H)-dione as the terminal group. Compound 169 thus produced was identified by ¹H NMR (CDCl₃, 500 MHz). (Blue solid, Yield=36%) ¹H NMR (500 MHz, CDCl₃) 8.95 (s, 4H), 8.50 (m, 4H), 8.46 (s, 2H), 8.25 (d, 4H), 7.86 (d, 4H), 7.27 (s, 4H), 3.16-3.11 (m, 2H), 2.59-2.52 (m, 4H), 1.50 (d, 12H), 1.22-1.14 (dd, 24H).

### Synthesis of Compounds 163 and 170

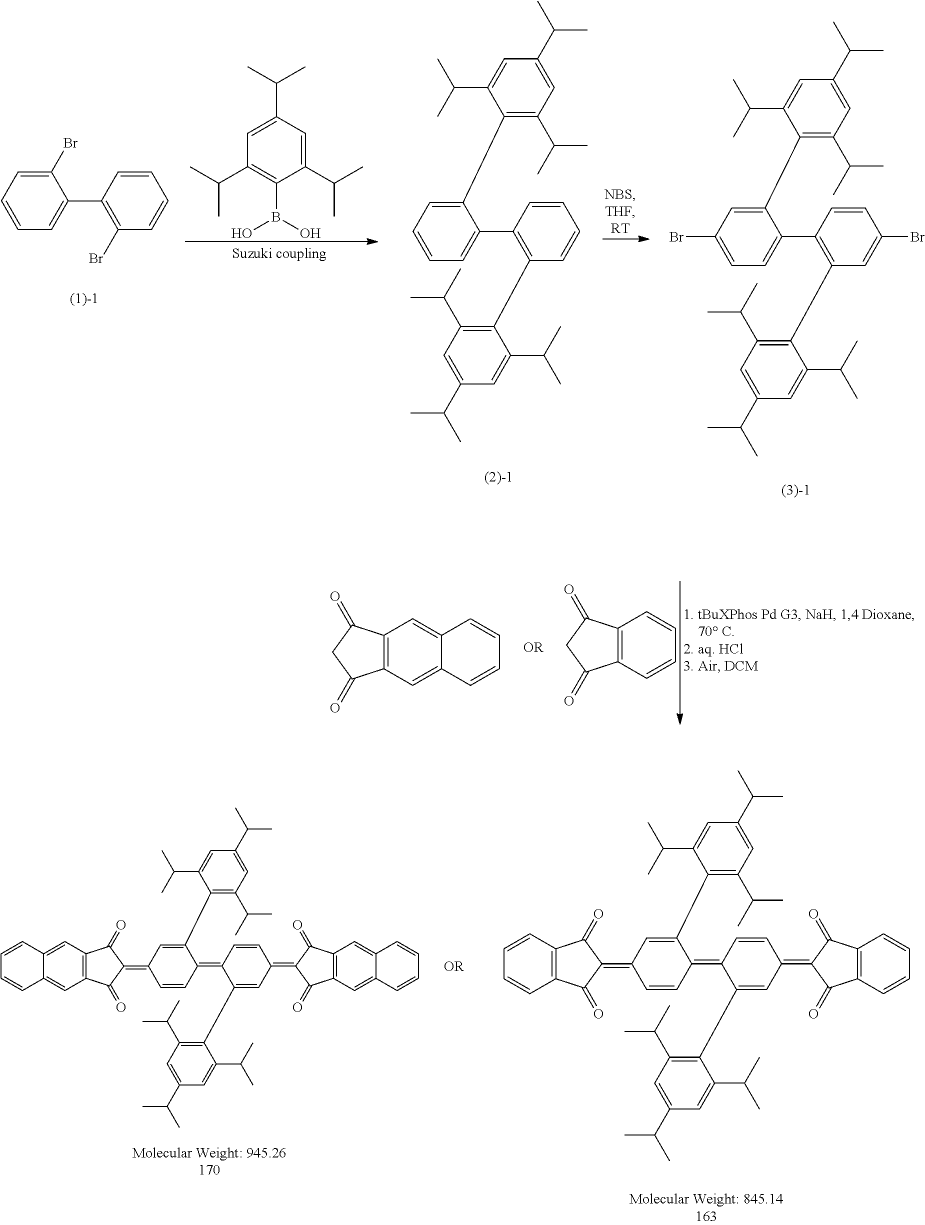

### Synthesis of Intermediate (2)-1 (2,2″′,4,4″′,6,6″′-hexaisopropyl-1,1′:2′,1″:2″,1″′-quaterphenyl)

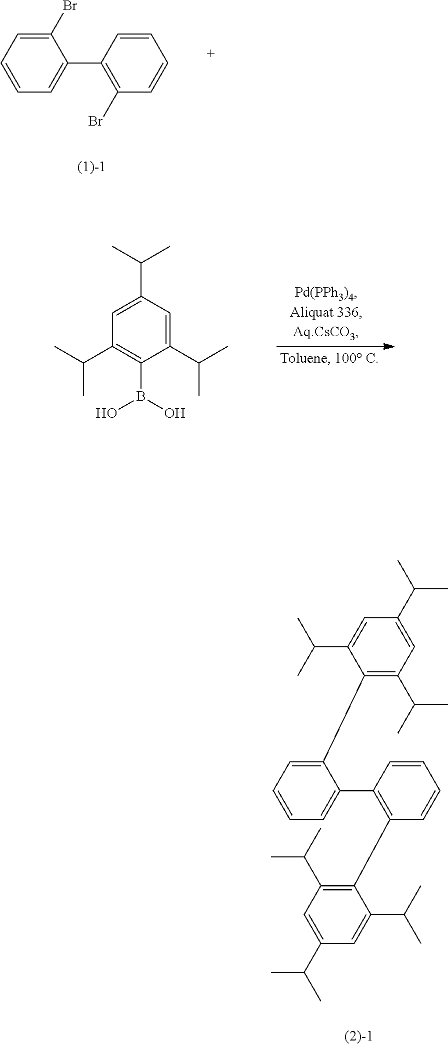

Intermediate (1)-1 (200 mg, 0.64 mmol), 2,4,6 tri-isopropyl phenyl boronic acid (636.3 mg, 2.56 mmol), and Aliquot 336 (3 drops) were added to a microwave vial, and then, Pd(PPh₃)₄ (34.7 mg, 0.03 mmol) was added thereto in a glove box. After anhydrous toluene (5 mL) and 2 M CsCO₃ (3 mL) were added thereto and then dissolved, the resultant mixture was stirred at 100° C. for 12 hours. After progress of the reaction was confirmed by TLC, the solvent was removed therefrom using a rotary evaporator, and an extraction process was performed thereon using diethyl ether and H₂O. After remaining moisture was removed therefrom using MgSO₄, the solvent of the filtered solution was removed using a rotary evaporator. Then, the resultant was separated by column chromatography to obtain Intermediate (2)-1. ¹H NMR (500 MHz, CDCl₃) 7.94 (s, 2H), 7.73 (m, 2H), 7.61 (m, 4H), 7.31 (s, 4H) 3.20-3.14 (m, 2H), 2.62-2.56 (m, 4H), 1.53 (d, 12H), 1.22-1.17 (dd, 24H).

### Synthesis of Intermediate (3)-1 (4″,5′-dibromo-2,2″′,4,4″′,6,6″′-hexaisopropyl-1,1′:2′,1″:2″,1″′-quaterphenyl)

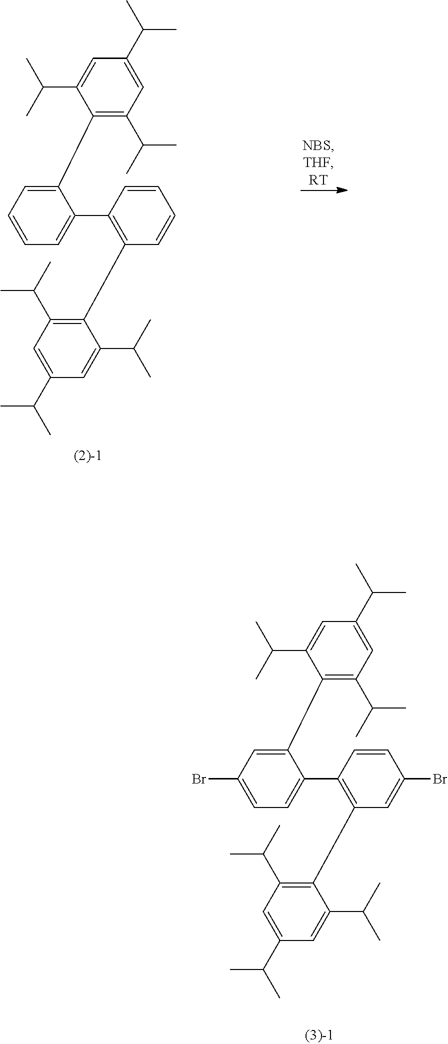

Intermediate (2)-1 (100 mg, 0.179 mmol) was dissolved in THF (150 mL), and then, the resultant mixture was stirred at room temperature for 10 minutes. NBS (38.2 mg, 0.214 mmol) was added thereto, and the mixture was stirred for 6 hours under nitrogen conditions while light was blocked with aluminum foil. After completion of the reaction, materials dissolved in organic solvents were extracted using DCM and H₂O. MgSO₄ was added thereto to remove moisture remaining after the extraction. After filtration was performed thereon, the solvent was removed therefrom by using a rotary evaporator. Then, the resultant was separated by column chromatography to obtain Intermediate (3)-1. ¹H NMR (500 MHz, CDCl₃) 7.92 (d, 2H), 7.83 (s, 2H), 7.63 (d, 2H), 7.31 (s, 4H) 3.20-3.14 (m, 2H), 2.62-2.56 (m, 4H), 1.53 (d, 12H), 1.22-1.17 (dd, 24H).

### Synthesis of Compound 163 (Q-IPBP-INDO, 2,2′-(2,2′-bis(2,4,6-triisopropylphenyl)-[1,1′-bi(cyclohexylidene)]-2,2′,5,5′-tetraenylidene-4,4′-diylidene)bis(1H-indene-1,3(2H)-dione)

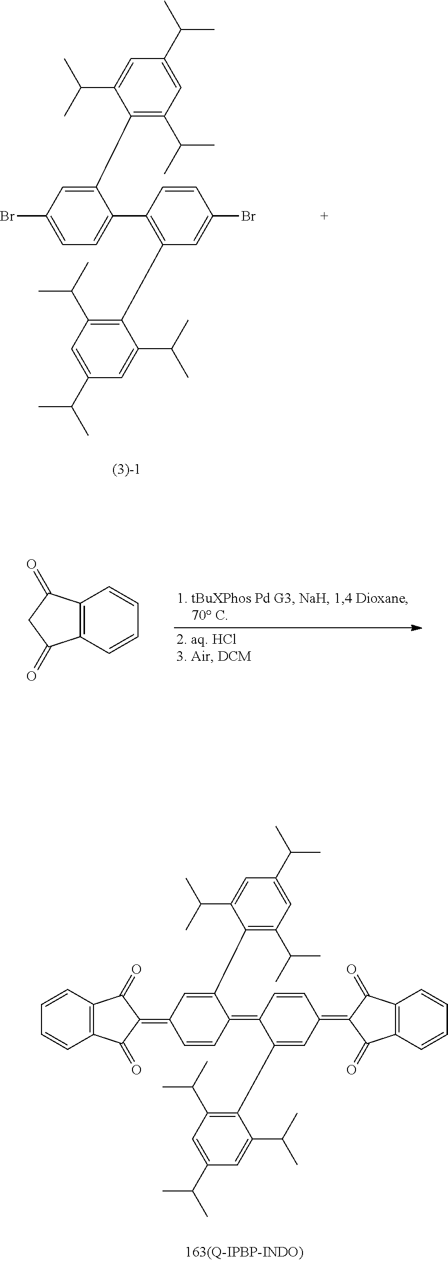 [SMILES: CC(C)c1cc(C(C)C)c(-c2cc(Br)ccc2-c2ccc(Br)cc2-c2c(C(C)C)cc(C(C)C)cc2C(C)C)c(C(C)C)c1 ; O=C1CC(=O)c2ccccc21 ; CC(C)c1cc(C(C)C)c(-c2cc(=C3C(=O)c4ccccc4C3=O)cc/c2=c2/ccc(=C3C(=O)c4ccccc4C3=O)cc2-c2c(C(C)C)cc(C(C)C)cc2C(C)C)c(C(C)C)c1]

After Intermediate (3)-1 (100 mg, 0.139 mmol), 1H-indene-1,3(2H)-dione (159.9 mg, 1.09 mmol), and NaH (13.2 mg, 0.552 mmol) were added to a microwave vial, tBuXPhos Pd G3 (10.4 mg, 0.013 mmol) was added thereto in a glove box, which was then sealed. 1,4-dioxane (3 mL) was added thereto, and then, the resultant mixture was heated to 70° C. and stirred for 12 hours while light was blocked with aluminum foil. After completion of the reaction, the solvent was removed therefrom, and 15 mL of 0.1 M HCl was added thereto, followed by stirring for 10 minutes. After completion of the reaction, materials dissolved in organic solvents were extracted using DCM and H₂O. MgSO₄ was added thereto, and then, the mixture was stirred in air for 20 minutes. Then, the resultant was separated by column chromatography to obtain Compound 163 (Q-IPBP-INDO). Compound 163 thus produced was identified by ¹H NMR (CDCl₃, 500 MHz). (Blue solid, Yield=36%)¹H NMR (500 MHz, CDCl₃) 7.84-7.79 (m, 4H), 7.68-7.64 (m, 4H), 7.27 (s, 4H), 6.51 (s, 2H), 5.94 (m, 4H), 3.16-3.11 (m, 2H), 2.59-2.52 (m, 4H), 1.50 (d, 12H), 1.22-1.14 (dd, 24H).

### Synthesis of Compound 170 (Q-IPBP-NPDO, 2,2′-(2,2′-bis(2,4,6-triisopropylphenyl)-[1,1′-bi(cyclohexylidene)]-2,2′,5,5′-tetraenylidene-4,4′-diylidene)bis(1H-cyclopenta[b]naphthalene-1,3(2H)-dione)

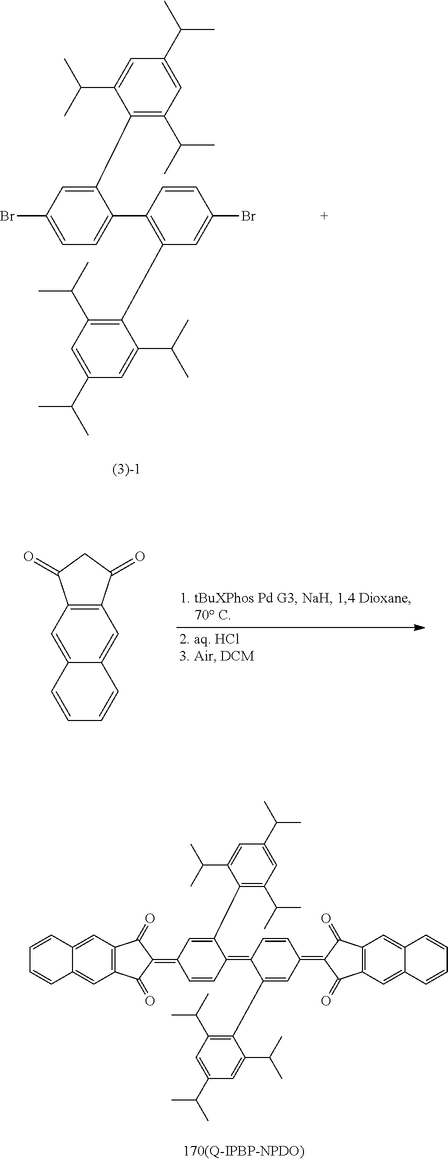 [SMILES: CC(C)c1cc(C(C)C)c(-c2cc(Br)ccc2-c2ccc(Br)cc2-c2c(C(C)C)cc(C(C)C)cc2C(C)C)c(C(C)C)c1 ; O=C1CC(=O)c2cc3ccccc3cc21 ; CC(C)c1cc(C(C)C)c(-c2cc(=C3C(=O)c4cc5ccccc5cc4C3=O)cc/c2=c2/ccc(=C3C(=O)c4cc5ccccc5cc4C3=O)cc2-c2c(C(C)C)cc(C(C)C)cc2C(C)C)c(C(C)C)c1]

Compound 170 was synthesized in substantially the same manner as used to synthesize Compound 163, except that 1H-cyclopenta[b]naphthalene-1,3(2H)-dione was used instead of 1H-indene-1,3(2H)-dione in Synthesis of Compound 163. Compound 170 thus produced was identified by ¹H NMR. (Blue solid, Yield=30%)¹H NMR (500 MHz, CDCl₃) 8.95 (s, 4H), 8.25 (d, 4H), 7.86 (d, 4H), 7.27 (s, 4H), 6.51 (s, 2H), 5.94 (m, 4H), 3.16-3.11 (m, 2H), 2.59-2.52 (m, 4H), 1.50 (d, 12H), 1.22-1.14 (dd, 24H).

### Synthesis of Compounds 171 and 172

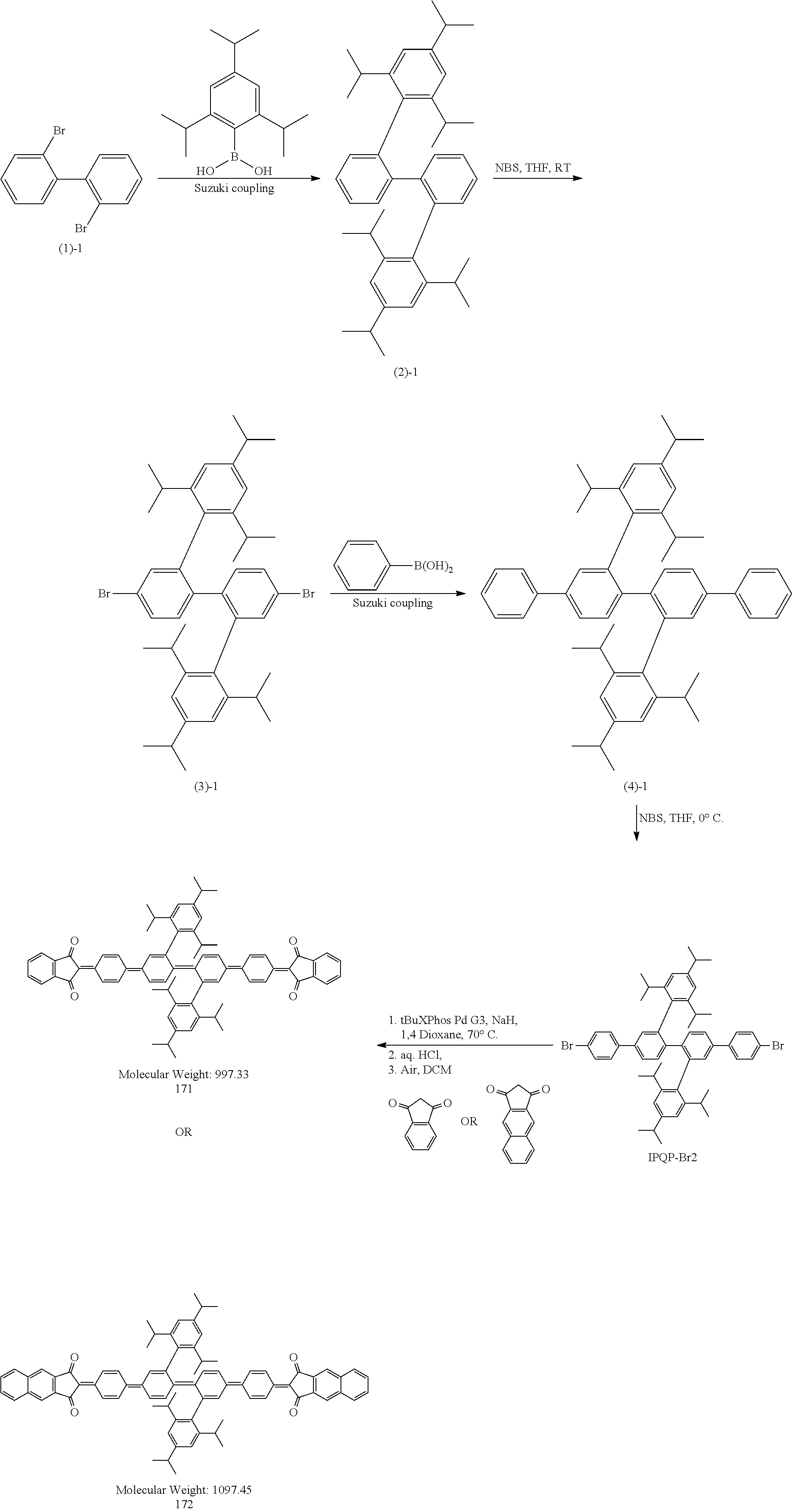

### Synthesis of Intermediate (4)-1 (2,2″′,4,4″′,6,6″′-hexaisopropyl-1,1′:2′,1″:2″,1″′-quaterphenyl)

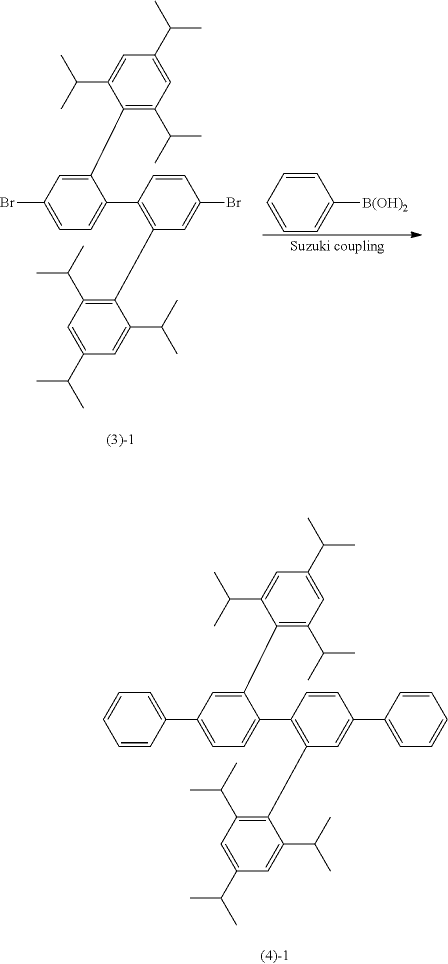

Intermediate (3)-1 (300 mg, 0.42 mmol) and phenylboronic acid (123.95 mg, 1.02 mmol) were added to a microwave vial, and then, Pd(dppf)₂Cl₂ (29.2 mg, 0.04 mmol) was added thereto in a glove box. After anhydrous toluene (4 mL), anhydrous THF (2 mL), and 2M Na₂CO₃ (3 mL) were added thereto and then dissolved, the resultant mixture was stirred at 100° C. for 12 hours. After completion of the reaction, the solvent was removed therefrom using a rotary evaporator, and an extraction process was performed thereon using diethyl ether and H₂O. After remaining moisture was removed therefrom using MgSO₄, the solvent of the filtered solution was removed using a rotary evaporator. Then, the resultant was separated by column chromatography to obtain Intermediate (4)-1. ¹H NMR (500 MHz, CDCl₃) 8.15 (d, 2H), 8.13 (s, 2H), 7.79 (d, 4H), 7.46 (m, 4H), 7.41 (m, 2H), 7.35 (d, 2H), 7.31 (s, 4H), 3.20-3.14 (m, 2H), 2.62-2.56 (m, 4H), 1.53 (d, 12H), 1.22-1.17 (dd, 24H).

### Synthesis of Intermediate (5)-1 (4″,5′-bis(4-bromophenyl)-2,2″′,4,4″′,6,6″′-hexaisopropyl-1,1′:2′,1″:2″,1″′-quaterphenyl)

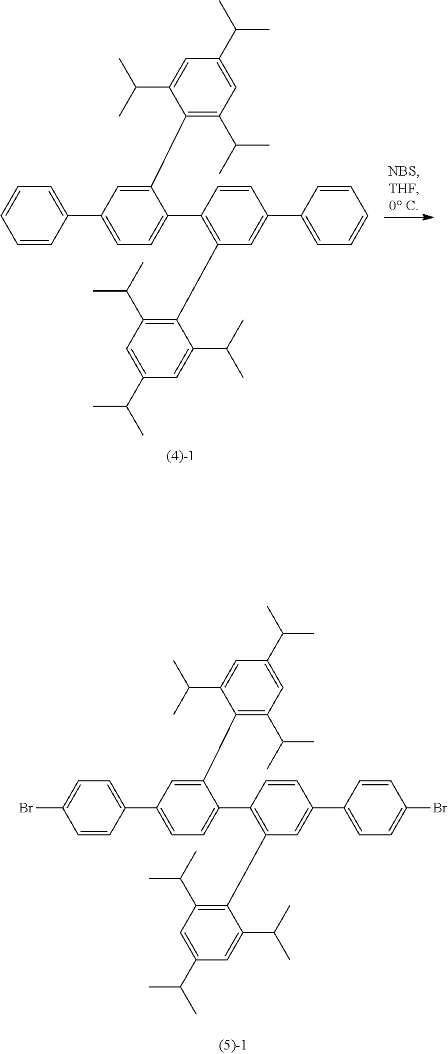

Intermediate (4)-1 (200 mg, 0.28 mmol) was dissolved in THF (250 mL), and then, the resultant mixture was stirred at 0° C. for 10 minutes. NBS (96.1 mg, 0.54 mmol) was added thereto, and the mixture was stirred at 0° C. for 15 minutes under nitrogen conditions while light was blocked with aluminum foil. After completion of the reaction, the solvent was removed therefrom using a rotary evaporator, and then, materials dissolved in organic solvents were extracted using DCM and H₂O. Moisture remaining after the extraction was removed by adding MgSO₄ thereto, and then, the solvent was removed from the filtered solution by using a rotary evaporator. Then, the resultant was separated by column chromatography to obtain Intermediate (5)-1. ¹H NMR (500 MHz, CDCl₃) 8.15 (d, 2H), 8.13 (s, 2H), 7.56 (d, 4H), 7.53 (d, 4H), 7.35 (d, 2H), 7.31 (s, 4H), 3.20-3.14 (m, 2H), 2.62-2.56 (m, 4H), 1.53 (d, 12H), 1.22-1.17 (dd, 24H).

### Synthesis of Compound 171 (Q-IPQP-INDO, 2,2′-(2″,3′-bis(2,4,6-triisopropylphenyl)-[1,1′:4′,1″:4″,1″′-quatercyclohexan]-1(1′),1″(4′),1″′(4″),2,2′,2″,2″′,5,5′,5″,5″′-undecaene-4,4″′-diylidene) bis(1H-indene-1,3(2H)-dione)

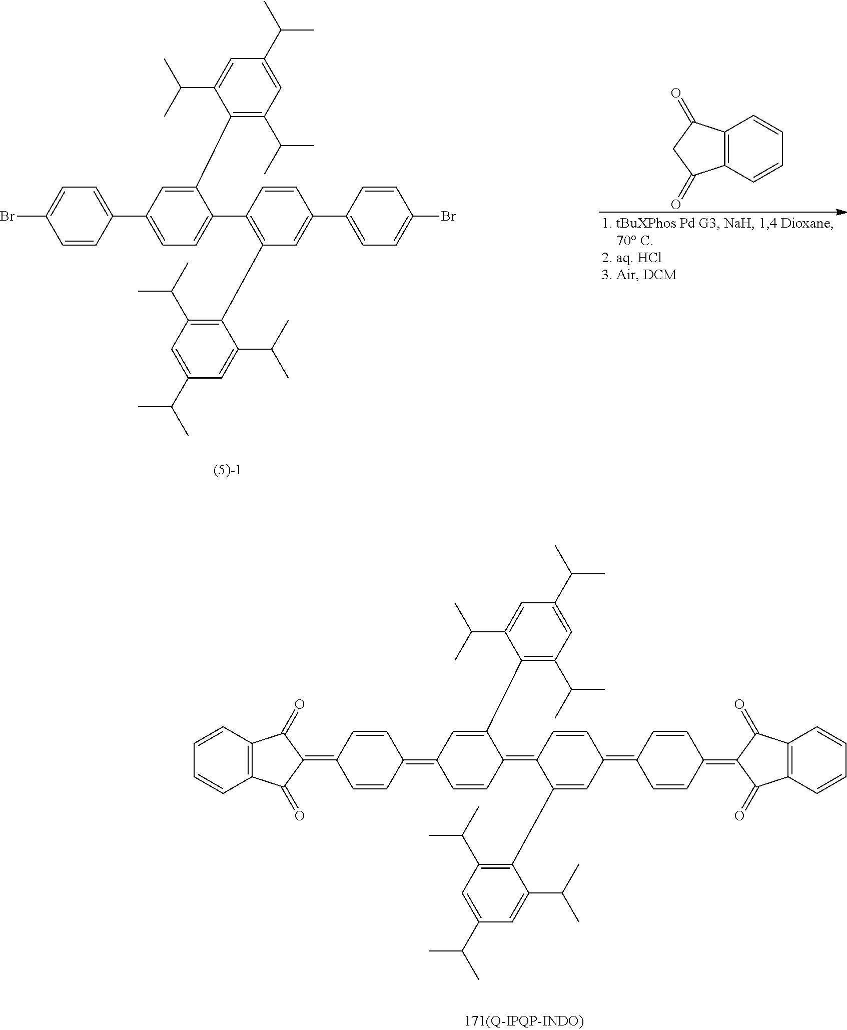 [SMILES: CC(C)c1cc(C(C)C)c(-c2cc(-c3ccc(Br)cc3)ccc2-c2ccc(-c3ccc(Br)cc3)cc2-c2c(C(C)C)cc(C(C)C)cc2C(C)C)c(C(C)C)c1 ; O=C1CC(=O)c2ccccc21 ; CC(C)c1cc(C(C)C)c(-c2cc(=c3ccc(=C4C(=O)c5ccccc5C4=O)cc3)cc/c2=c2/ccc(=c3ccc(=C4C(=O)c5ccccc5C4=O)cc3)cc2-c2c(C(C)C)cc(C(C)C)cc2C(C)C)c(C(C)C)c1]

Compound 171 was synthesized in substantially the same manner as used to synthesize Compound 163, except that Intermediate (5)-1 was used instead of Intermediate (3)-1 in Synthesis of Compound 163. Compound 171 thus produced was identified by ¹H NMR. (Blue solid, Yield=34%)¹H NMR (500 MHz, CDCl₃) 7.84-7.79 (m, 4H), 7.68-7.64 (m, 4H), 7.27 (s, 4H), 6.51 (s, 2H), 5.94 (m, 12H), 3.16-3.11 (m, 2H), 2.59-2.52 (m, 4H), 1.50 (d, 12H), 1.22-1.14 (dd, 24H).

### Synthesis of Compound 172 (Q-IPQP-NPDO, 2,2′-(2″,3′-bis(2,4,6-triisopropylphenyl)-[1,1′:4′,1″:4″,1″′-quatercyclohexan]-1 (1′),1″(4′),1″′(4″),2,2′,2″,2″′,5,5′,5″,5″′-undecaene-4,4″′-diylidene)bis(1H-cyclopenta[b]naphthalene-1,3(2H)-dione)

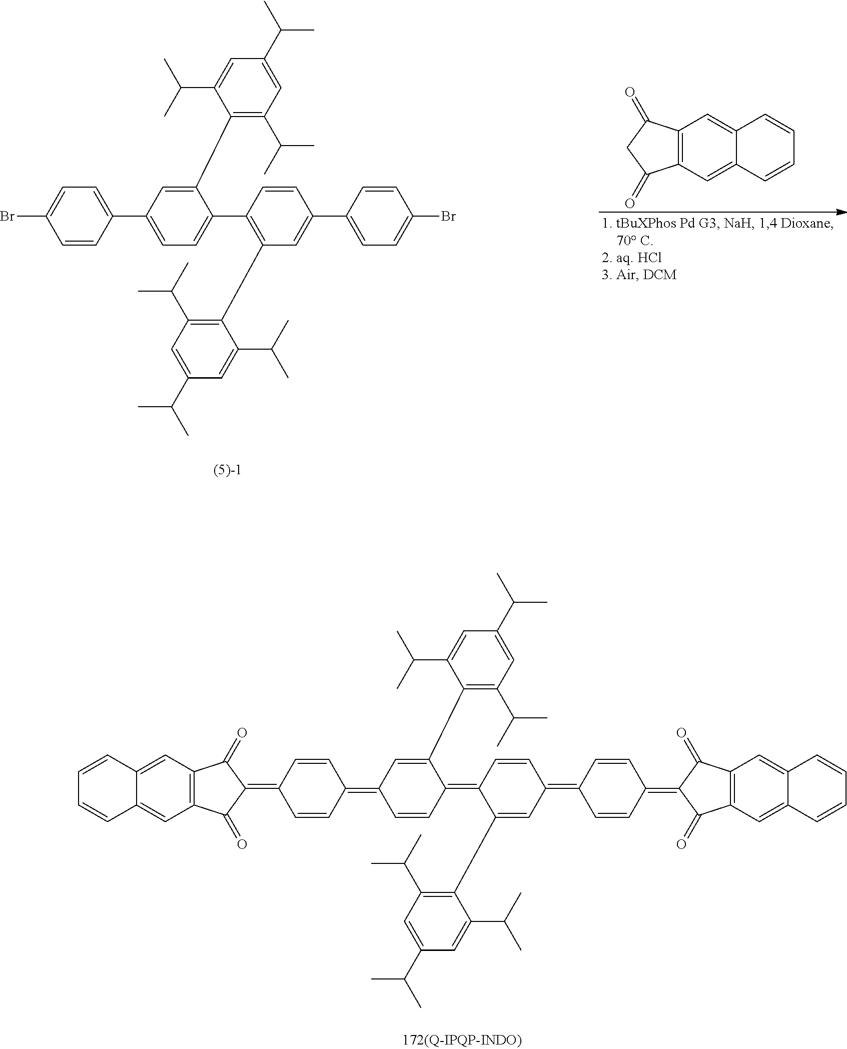 [SMILES: CC(C)c1cc(C(C)C)c(-c2cc(-c3ccc(Br)cc3)ccc2-c2ccc(-c3ccc(Br)cc3)cc2-c2c(C(C)C)cc(C(C)C)cc2C(C)C)c(C(C)C)c1 ; O=C1CC(=O)c2cc3ccccc3cc21 ; CC(C)c1cc(C(C)C)c(-c2cc(=c3ccc(=C4C(=O)c5cc6ccccc6cc5C4=O)cc3)cc/c2=c2/ccc(=c3ccc(=C4C(=O)c5cc6ccccc6cc5C4=O)cc3)cc2-c2c(C(C)C)cc(C(C)C)cc2C(C)C)c(C(C)C)c1]

Compound 172 was synthesized in substantially the same manner as used to synthesize Compound 163, except that, in Synthesis of Compound 163, Intermediate (5)-1 was used instead of Intermediate (3)-1, and 1H-cyclopenta[b]naphthalene-1,3(2H)-dione was used instead of 1H-indene-1,3(2H)-dione as the terminal group. Compound 172 thus produced was identified by ¹H NMR. (Blue solid, Yield=32%)¹H NMR (500 MHz, CDCl₃) 8.95 (s, 4H), 8.25 (d, 4H), 7.86 (d, 4H), 7.27 (s, 4H), 6.51 (s, 2H), 5.94 (m, 12H), 3.16-3.11 (m, 2H), 2.59-2.52 (m, 4H), 1.50 (d, 12H), 1.22-1.14 (dd, 24H).

### Absorption Wavelength, HOMO Energy Level, LUMO Energy Level, and Band Gap

Absorption wavelengths, HOMO energy levels, LUMO energy levels, decomposition temperatures, band gaps, and extinction coefficients of Compounds 132, 140, 141, 143, 163, 168, 169 to 172, and B-IPBT-INDO were measured, and results thereof are shown in Table 1.

The HOMO energy levels were measured using cyclic voltammetry (CV). The absorption wavelengths, LUMO energy levels, band gaps, and extinction coefficients were measured using UV-vis spectroscopy. The decomposition temperatures were measured using a thermogravimetric analyzer (TGA).

TABLE 1

| | λₘₐₓ | | | | | ε × 10⁵ |
|---|---|---|---|---|---|---|
| | (film) | HOMO^{CV} | LUMO^{OPT} | E_{g}^{opt} | T_{d} | (cm⁻¹) |
| | [nm] | (eV) | (eV) | (eV) | (° C.) | @ λₘₐₓ |
| Compound | 676 | −5.58 | −3.89 | 1.69 | 290 | 4.83 |
| 132 | | | | | | |
| Compound | 666 | −5.64 | −3.93 | 1.71 | 261 | 3.01 |
| 140 | | | | | | |
| Compound | 670 | −5.68 | −3.97 | 1.71 | 255 | 2.77 |
| 141 | | | | | | |
| Compound | 743 | −5.74 | −4.21 | 1.53 | 296 | 4.10 |
| 168 | | | | | | |
| Compound | 883 | −4.89 | −3.96 | 0.93 | 317 | 3.78 |
| 143 | | | | | | |
| Compound | 714 | −5.52 | −4.3 | 1.22 | 279 | 3.42 |
| 163 | | | | | | |
| Compound | 958 | −5.08 | −4.29 | 0.79 | 282 | 3.11 |
| 169 | | | | | | |
| Compound | 789 | −5.68 | −4.61 | 1.07 | 287 | 2.76 |
| 170 | | | | | | |
| Compound | 921 | −4.83 | −4.37 | 0.46 | 304 | 2.68 |
| 171 | | | | | | |
| Compound | 1004 | −5.02 | −4.69 | 0.33 | 273 | 2.09 |
| 172 | | | | | | |
| B-IPBT-INDO | 569 | −5.84 | −3.83 | 2.01 | 287 | 1.32 |

λₘₐₓ(film): maximum wavelength of absorbed light measured by preparing compound as film of 300 Å
HOMO^{CV}: HOMO energy level calculated from oxidation onset of CV
LUMO^{OPT}: LUMO energy level calculated from formula HOMO^{CV}+E_{g}^{opt} (eV)
E_{g}^{opt}(eV): optical band gap
Td (° C.): decomposition temperature
ε×10⁵ (cm⁻¹) @ λₘₐₓ: extinction coefficient at maximum wavelength

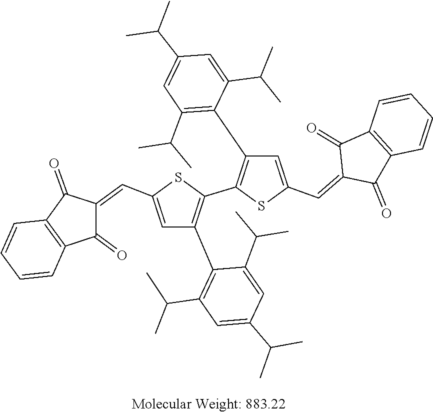 [SMILES: CC(C)c1cc(C(C)C)c(-c2cc(C=C3C(=O)c4ccccc4C3=O)sc2-c2sc(C=C3C(=O)c4ccccc4C3=O)cc2-c2c(C(C)C)cc(C(C)C)cc2C(C)C)c(C(C)C)c1]

### B-IPBT-INDO

B-IPBT-INDO, which is a benzenoid compound, has a structure similar to that of Compound 132. However, referring to Table 1, it may be seen that, although Compound 132 has a smaller molecular weight than B-IPBT-INDO, which is a benzenoid compound, Compound 132 absorbs a longer wavelength and has a shorter band gap than B-IPBT-INDO.

### Comparative Example 1

An ITO glass substrate (anode) was cut to a size of 50 mm×50 mm×0.5 mm, ultrasonically cleaned with isopropyl alcohol and pure water each for 15 minutes, and then cleaned by irradiation of ultraviolet rays and exposure to ozone for 10 minutes. Then, the ITO substrate was loaded into a vacuum deposition apparatus. HAT-CN was vacuum-deposited on the anode to form a hole injection layer having a thickness of 100 Å, and HT3 was vacuum-deposited on the hole injection layer to form a hole transport layer having a thickness of 1,250 Å.

m-MTDATA was vacuum-deposited on the hole transport layer to form an auxiliary layer having a thickness of 200 Å.

B-IPBT-INDO and N14 were sequentially deposited to thicknesses of 80 Å and 340 Å, respectively, on the auxiliary layer to form an active layer.

Then, BAlq was vacuum-deposited thereon to form a buffer layer having a thickness of 50 Å, and ET1 was vacuum-deposited on the buffer layer to form an electron transport layer having a thickness of 300 Å.

LiF was vacuum-deposited on the electron transport layer to form an electron injection layer having a thickness of 10 Å, and then, MgAg was sequentially deposited thereon to form a cathode having a thickness of 100 Å, thereby completing the manufacture of an organic photodetector.

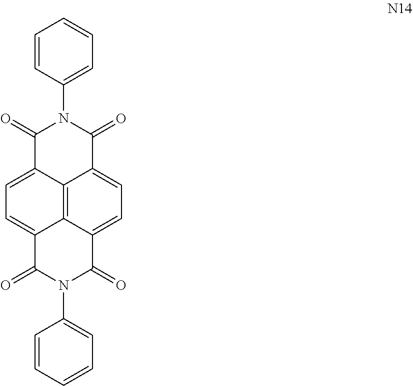 [SMILES: O=C1c2ccc3c4c(ccc(c24)C(=O)N1c1ccccc1)C(=O)N(c1ccccc1)C3=O]

### Example 1

An organic photodetector was manufactured in substantially the same manner as in Comparative Example 1, except that, in forming the active layer, Compound 132 and N14 were deposited to thicknesses of 80 Å and 340 Å, respectively, to form an active layer.

### Example 2

An organic photodetector was manufactured in substantially the same manner as in Comparative Example 1, except that, in forming the active layer, Compound 140 was used instead of Compound 132.

### Example 3

An organic photodetector was manufactured in substantially the same manner as in Comparative Example 1, except that, in forming the active layer, Compound 141 was used instead of Compound 132.

### Example 4

An organic photodetector was manufactured in substantially the same manner as in Comparative Example 1, except that, in forming the active layer, Compound 168 was used instead of Compound 132.

### Example 5

An organic photodetector was manufactured in substantially the same manner as in Comparative Example 1, except that, in forming the active layer, Compound 143 was used instead of Compound 132.

### Example 6

An organic photodetector was manufactured in substantially the same manner as in Comparative Example 1, except that, in forming the active layer, Compound 163 was used instead of Compound 132.

### Example 7

An organic photodetector was manufactured in substantially the same manner as in Comparative Example 1, except that, in forming the active layer, Compound 169 was used instead of Compound 132.

### Example 8

An organic photodetector was manufactured in substantially the same manner as in Comparative Example 1, except that, in forming the active layer, Compound 170 was used instead of Compound 132.

### Example 9

An organic photodetector was manufactured in substantially the same manner as in Comparative Example 1, except that, in forming the active layer, Compound 171 was used instead of Compound 132.

### Example 10

An organic photodetector was manufactured in substantially the same manner as in Comparative Example 1, except that, in forming the active layer, Compound 172 was used instead of Compound 132.

External quantum efficiency (EQE) with respect to a wavelength in a range from 580 nm to 700 nm, which is a peak wavelength of each of the organic photodetectors manufactured in Comparative Example 1 and Examples 1 to 10, was measured, and results thereof are shown in Table 2.

TABLE 2

| | EQE (%) @ 580-700 nm |
|---|---|
| Comparative Example 1 | 24.1% |
| Example 1 | 47.0% |
| Example 2 | 29.6% |
| Example 3 | 27.5% |
| Example 4 | 43.2% |
| Example 5 | 37.5% |
| Example 6 | 36.7% |
| Example 7 | 33.3% |
| Example 8 | 31.8% |
| Example 9 | 29.3% |
| Example 10 | 24.5% |

Referring to Table 2, it can be seen that the organic photodetectors of Examples 1 to 10 showed superior EQE to those of Comparative Example 1 in the same device structure.

A compound according to an embodiment may be deposited while absorbing light in the near-infrared region, and an organic photodetector and an electronic apparatus that use the compound in an active layer may have excellent efficiency.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims, and equivalents thereof.

What is claimed is:

1. A compound represented by one selected from Formulae 1 to 4: 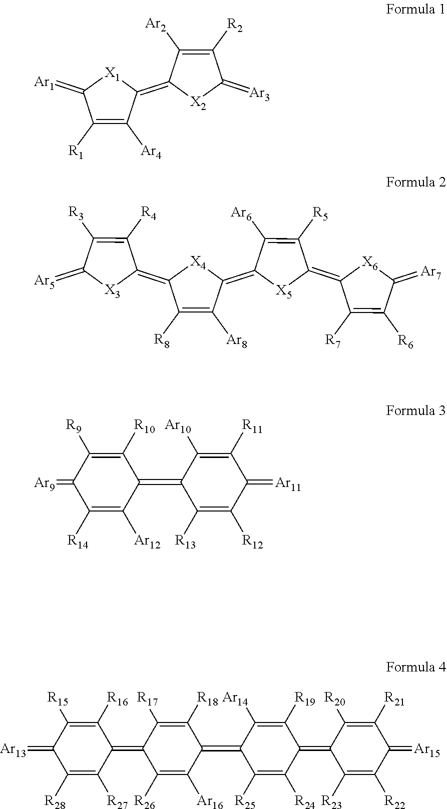
wherein, in Formulae 1 to 4,
X₁ to X₆ are each independently selected from O, S, Se, Te, CR₃₁R₃₂, NR₃₃, BR₃₄, SiR₃₅R₃₆, and GeR₃₇R₃₈,
Ar₁, Ar₃, Ar₅, Ar₇, Ar₉, Ar₁₁, Ar₁₃, and Ar₁₅ are each independently selected from Formulae 1-1 to 1-5, 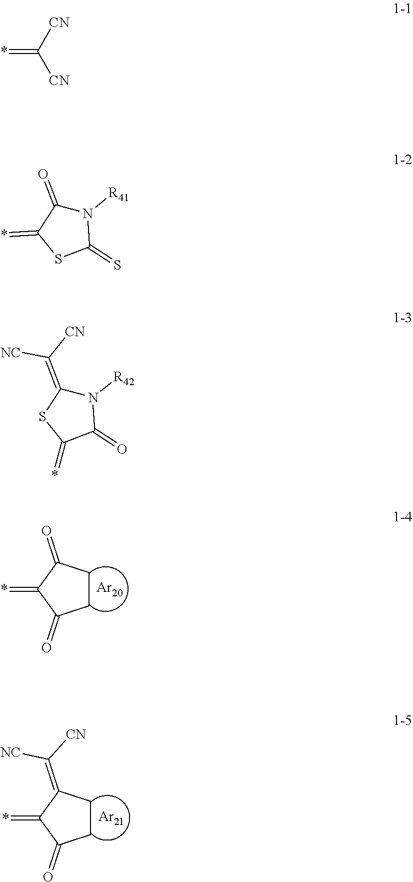
* indicates a bond with a neighboring atom,
Ar₂, Ar₄, Ar₆, Ar₈, Ar₁₀, Ar₁₂, Ar₁₄, Ar₁₆, Ar₂₀, and Ar₂₁ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
R₁ to R₂₈, R₃₁ to R₃₈, R₄₁, and R₄₂ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁a cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The compound of claim 1, wherein Ar₂, Ar₄, Ar₆, Ar₈, Ar₁₀, Ar₁₂, Ar₁₄, Ar₁₆, Ar₂₀, and Ar₂₁ are each independently hydrogen, deuterium, a C₆-C₆₀ aryl group unsubstituted or substituted with at least one R₁₀ₐ, or a C₁-C₆₀ heteroaryl group unsubstituted or substituted with at least one R₁₀ₐ, and
R₁₀ₐ is the same as defined in claim 1.

3. The compound of claim 1, wherein Ar₂, Ar₄, Ar₆, Ar₈, Ar₁₀, Ar₁₂, Ar₁₄, Ar₁₆, Ar₂₀, and Ar₂₁ are each independently selected from hydrogen, deuterium, and Formulae 2-1 to 2-8: 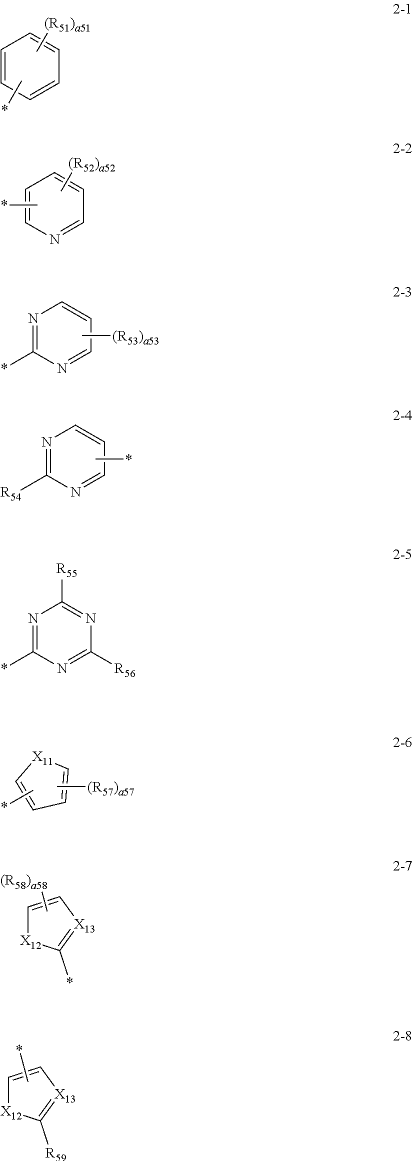
wherein, in Formulae 2-1 to 2-8, X₁₁ to X₁₃ each independently indicate N, O, or S, and R₅₁ to R₅₉ are each independently selected from hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
a51 is an integer from 1 to 5,
a52 is an integer from 1 to 4,
a53 and a57 are each independently an integer from 1 to 3,
a58 is 1 or 2, and
Q₁ to Q₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

4. The compound of claim 1, wherein R₁ to R₂₈ are each independently hydrogen or deuterium.

5. The compound of claim 1, wherein R₃₁ to R₃₈ are each independently hydrogen, deuterium, —F, —Cl, —Br, —I, a C₁-C₆₀ alkyl group, a C₁-C₆₀ alkoxy group, a C₆-C₆₀ aryl group, or a C₆-C₆₀ aryloxy group.

6. The compound of claim 1, wherein Ar₂₀ and Ar₂₁ are each independently a C₆-C₆₀ aryl group unsubstituted or substituted with at least one R₁₀ₐ, and R₁₀ₐ is the same as defined in claim 1.

7. The compound of claim 1, wherein R₄₁ and R₄₂ are each independently a C₁-C₆₀ alkyl group unsubstituted or substituted with at least one R₁₀ₐ or a C₆-C₆₀ aryl group unsubstituted or substituted with at least one R₁₀ₐ, and
R₁₀ₐ is the same as defined in claim 1.

8. The compound of claim 1, wherein the compound represented by one selected from Formulae 1 to 4 has C2 symmetry.

9. The compound of claim 1, wherein a molecular weight of the compound represented by one selected from Formulae 1 to 4 is 1,500 g/mol or less.

10. The compound of claim 1, wherein a highest occupied molecular orbital (HOMO) energy level of the compound represented by one selected from Formulae 1 to 4 is in a range of about −6.5 eV to about −4.5 eV, and a lowest unoccupied molecular orbital (LUMO) energy level of the compound represented by one selected from Formulae 1 to 4 is in a range of about −4.7 eV to about −3.0 eV.

11. The compound of claim 1, wherein an optical band gap of the compound represented by one selected from Formulae 1 to 4 is 2.0 eV or less.

12. The compound of claim 1, wherein a decomposition temperature of the compound represented by one selected from Formulae 1 to 4 is 200° C. or higher.

13. The compound of claim 1, wherein the compound represented by one selected from Formulae 1 to 4 is selected from compounds below:

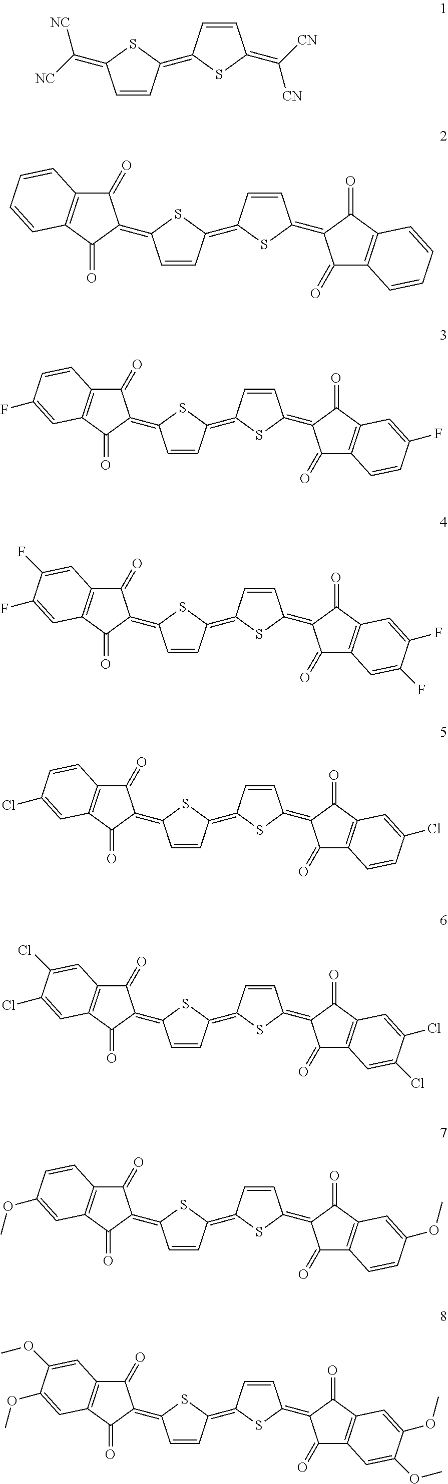

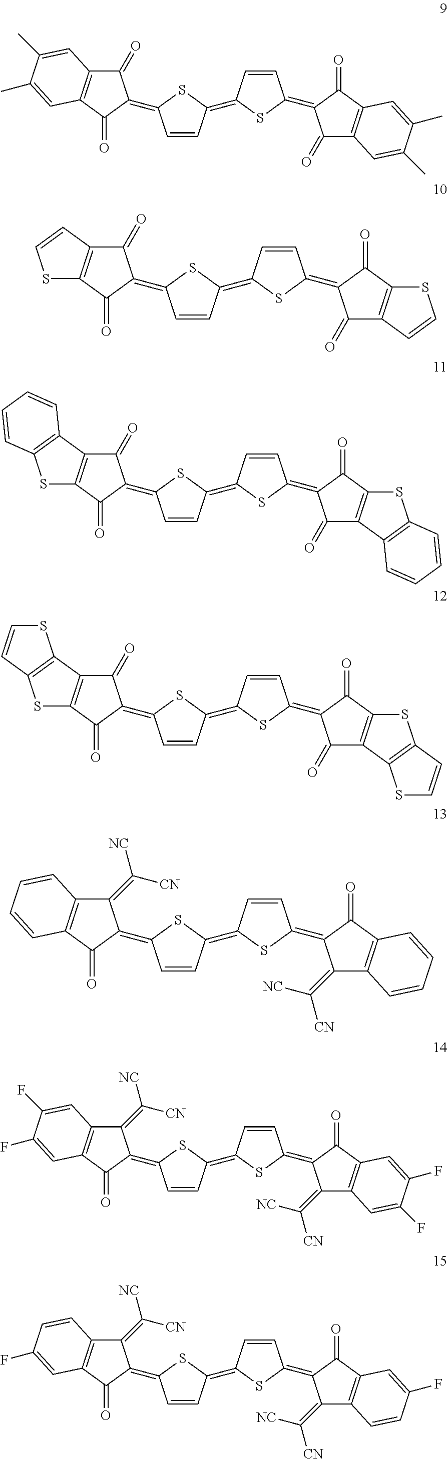

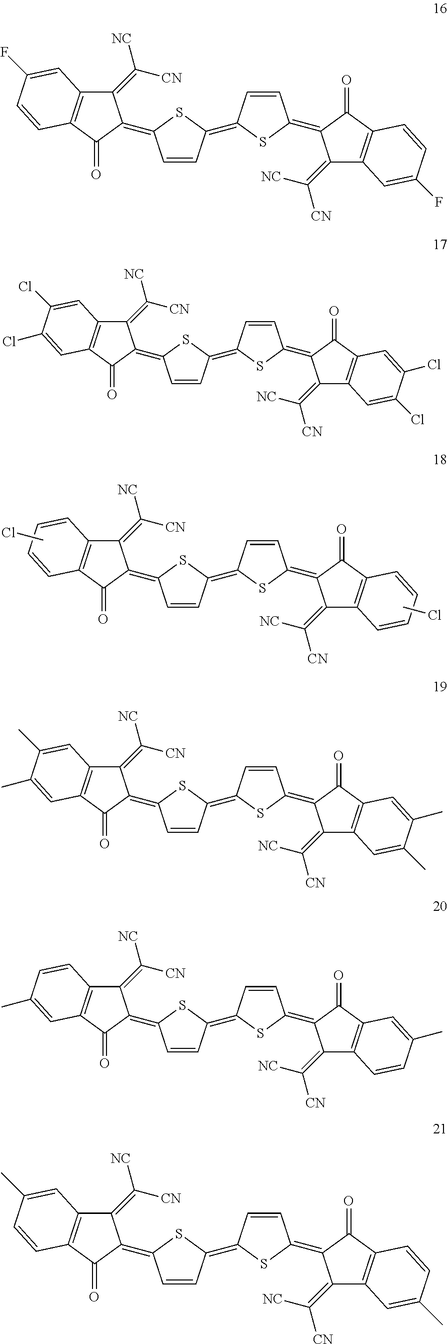

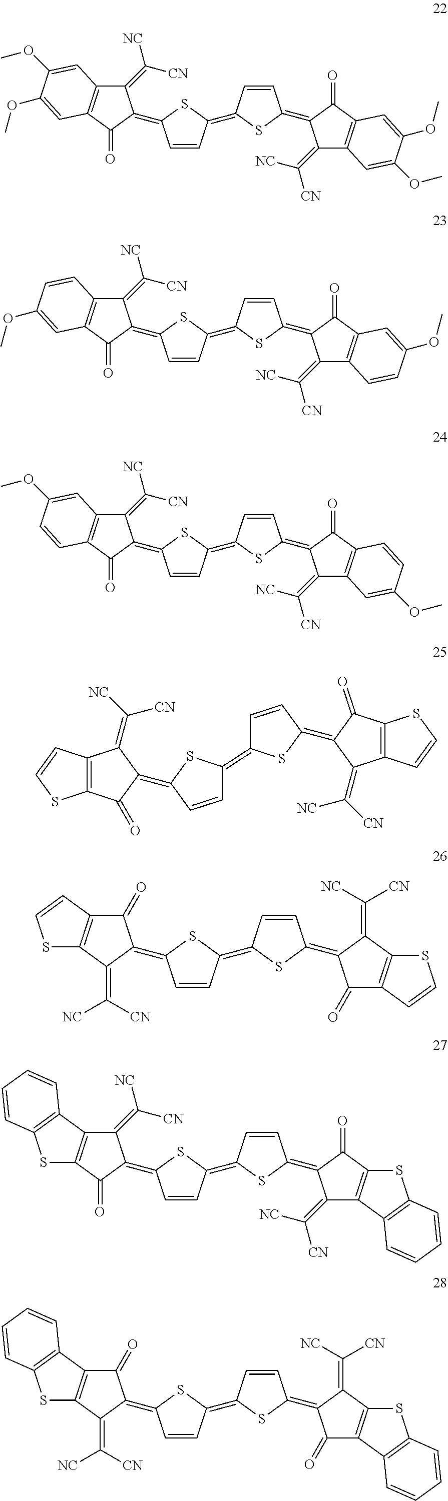

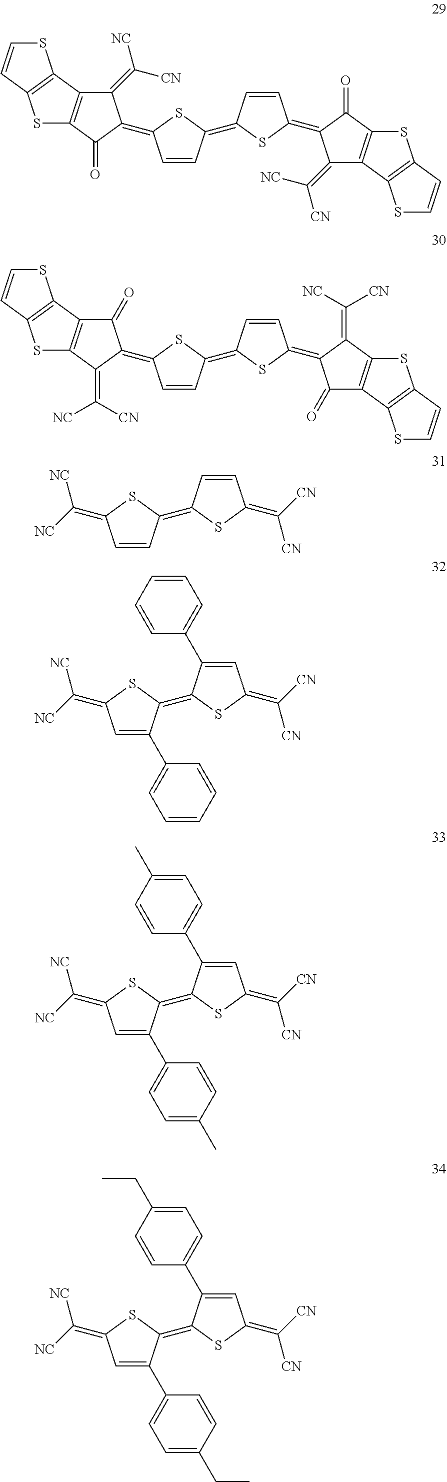

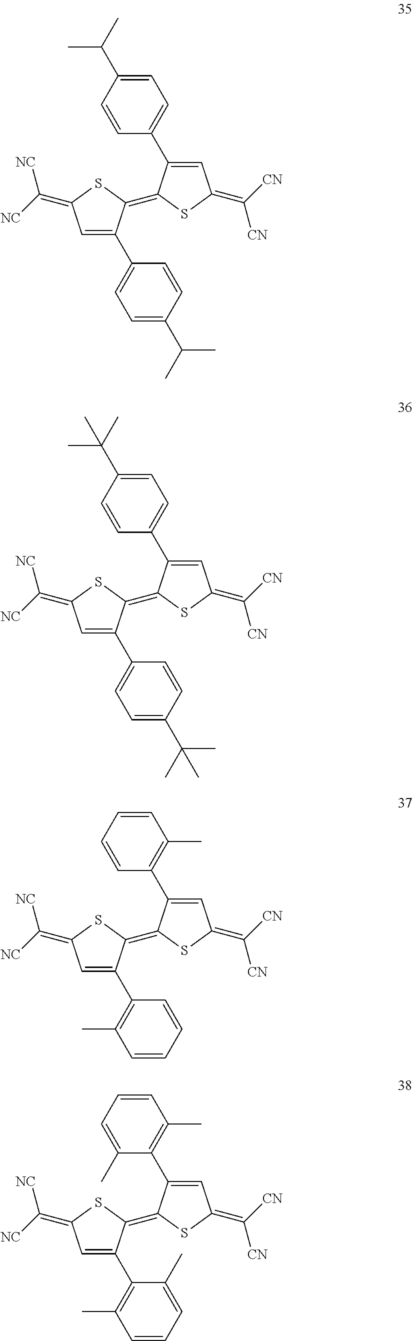

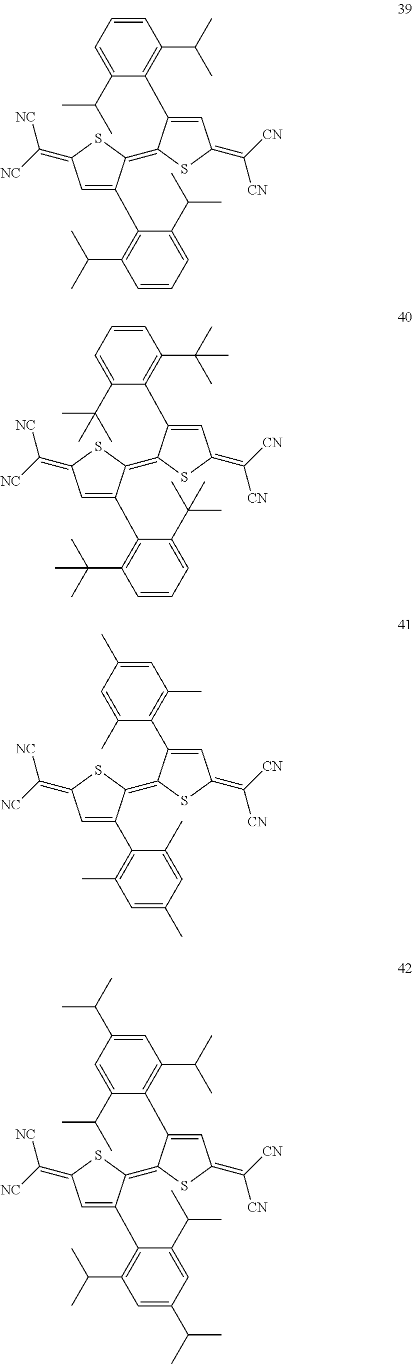

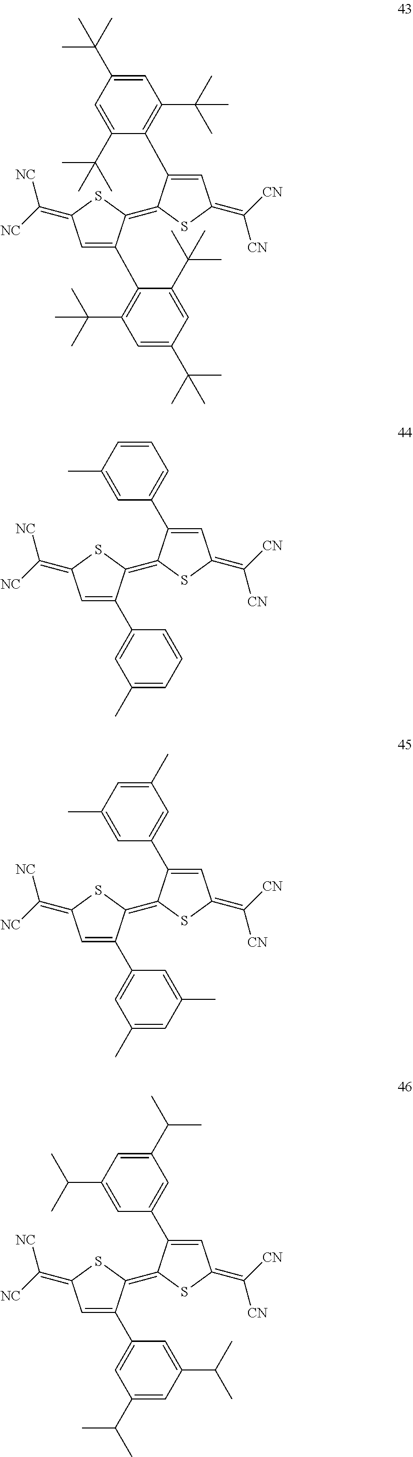

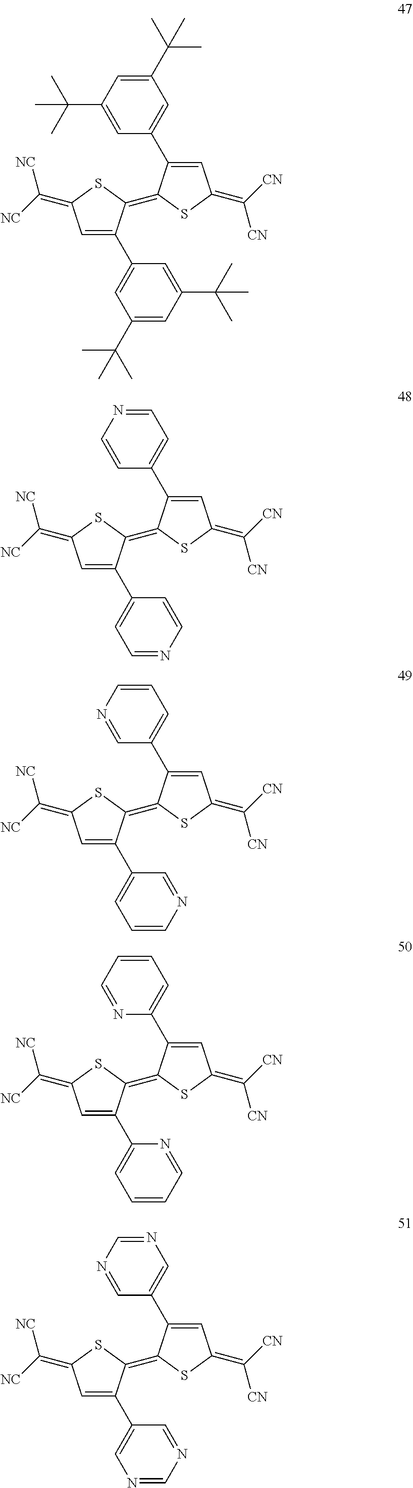

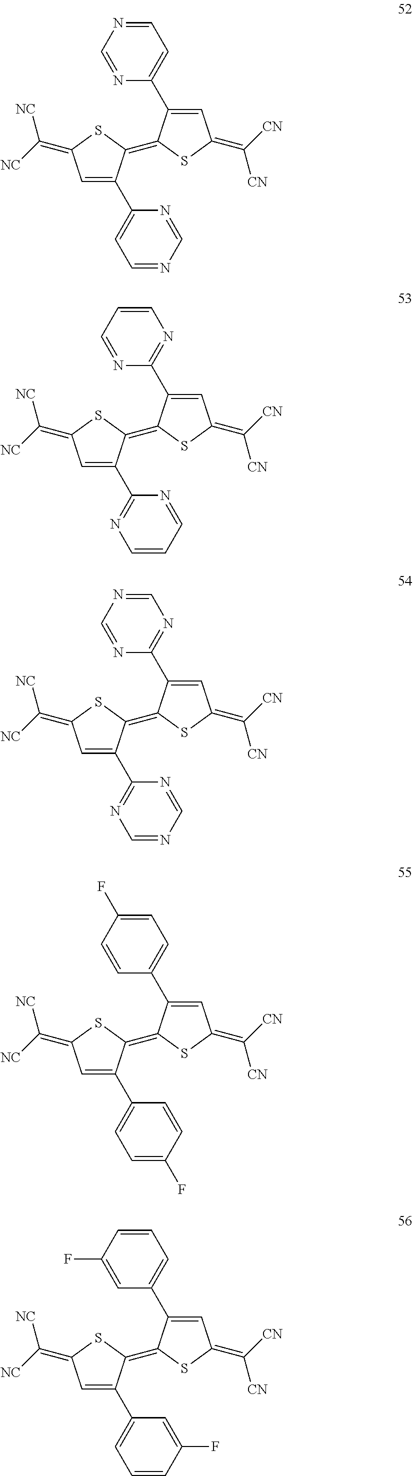

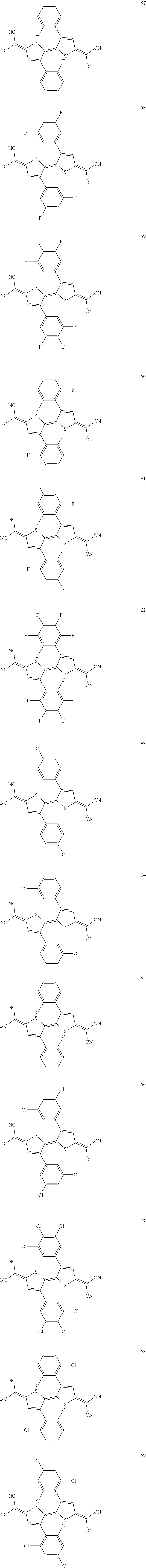

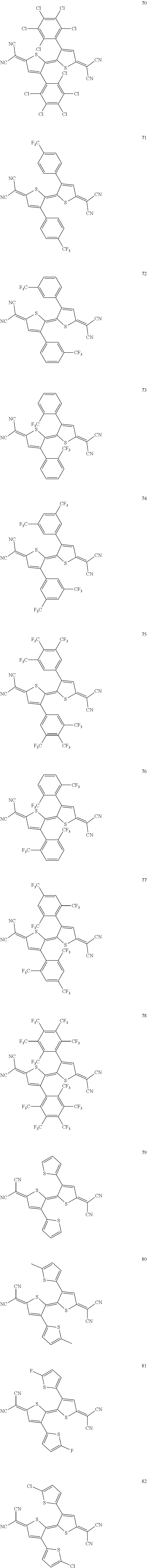

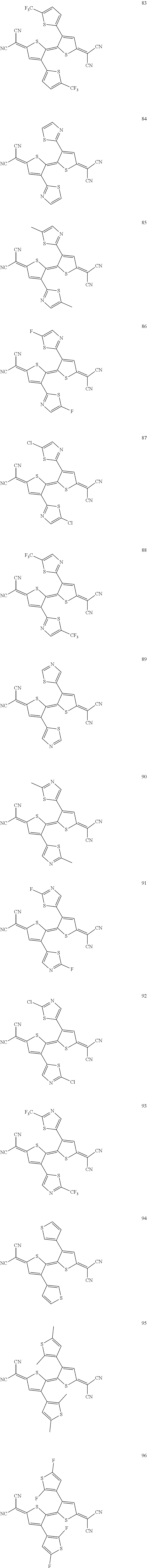

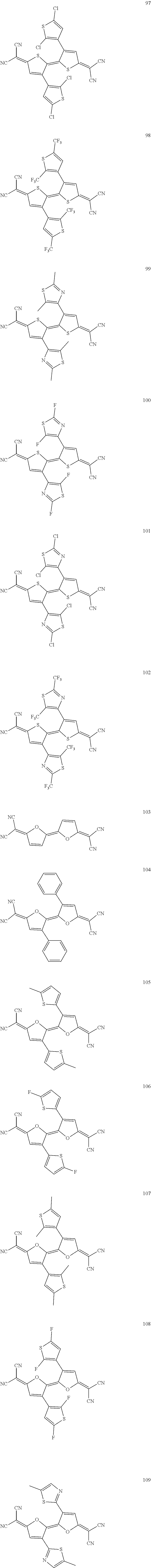

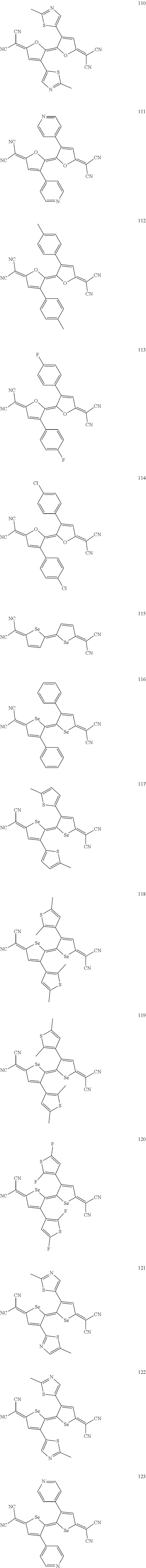

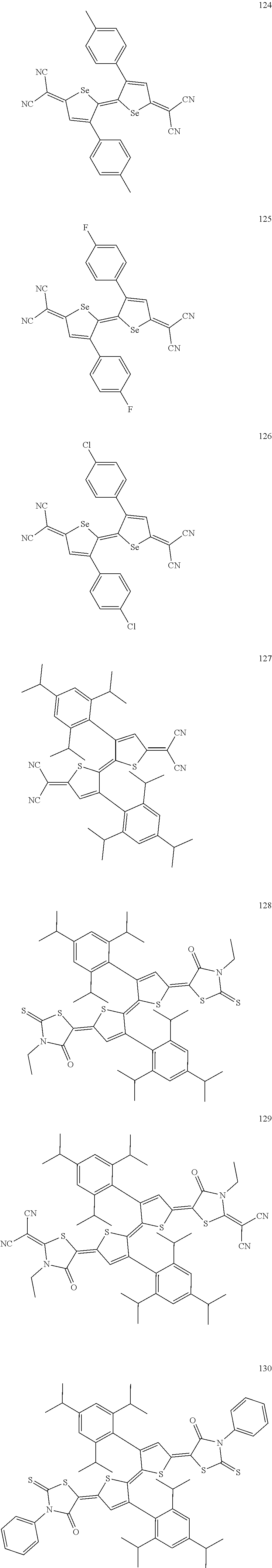

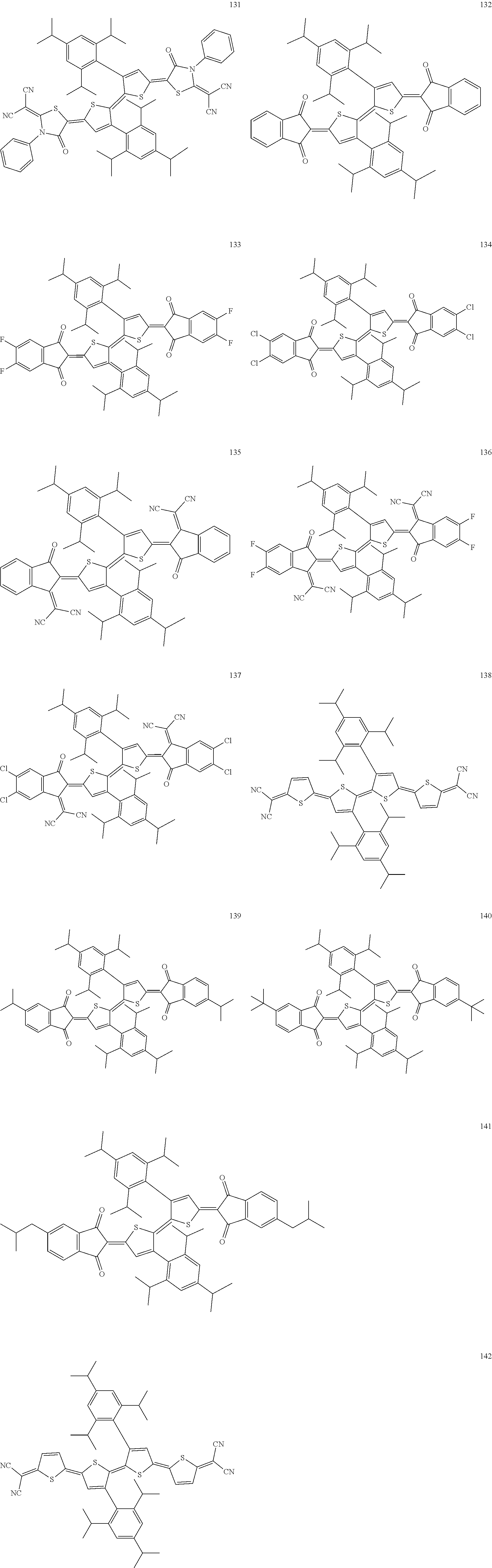

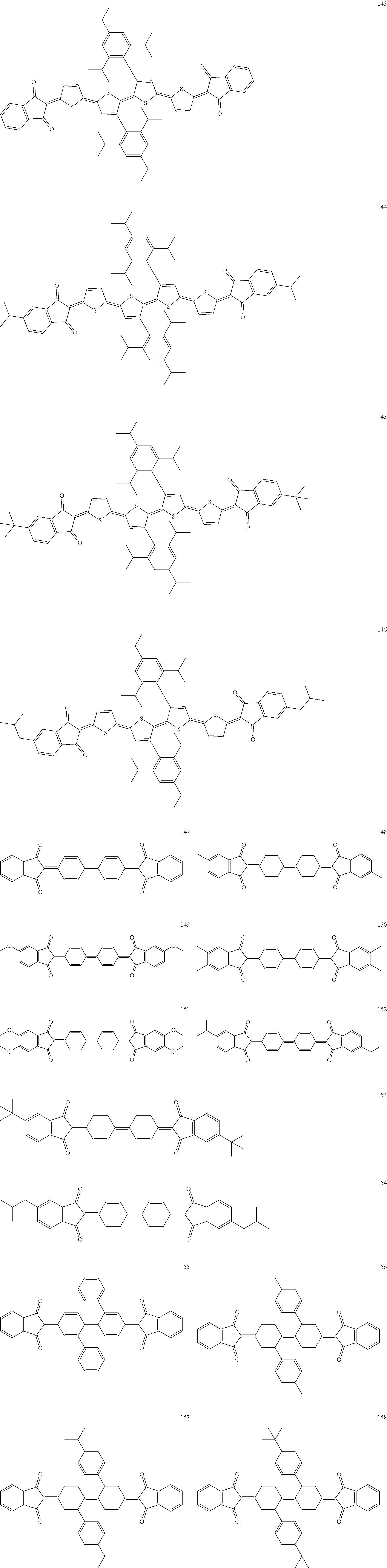

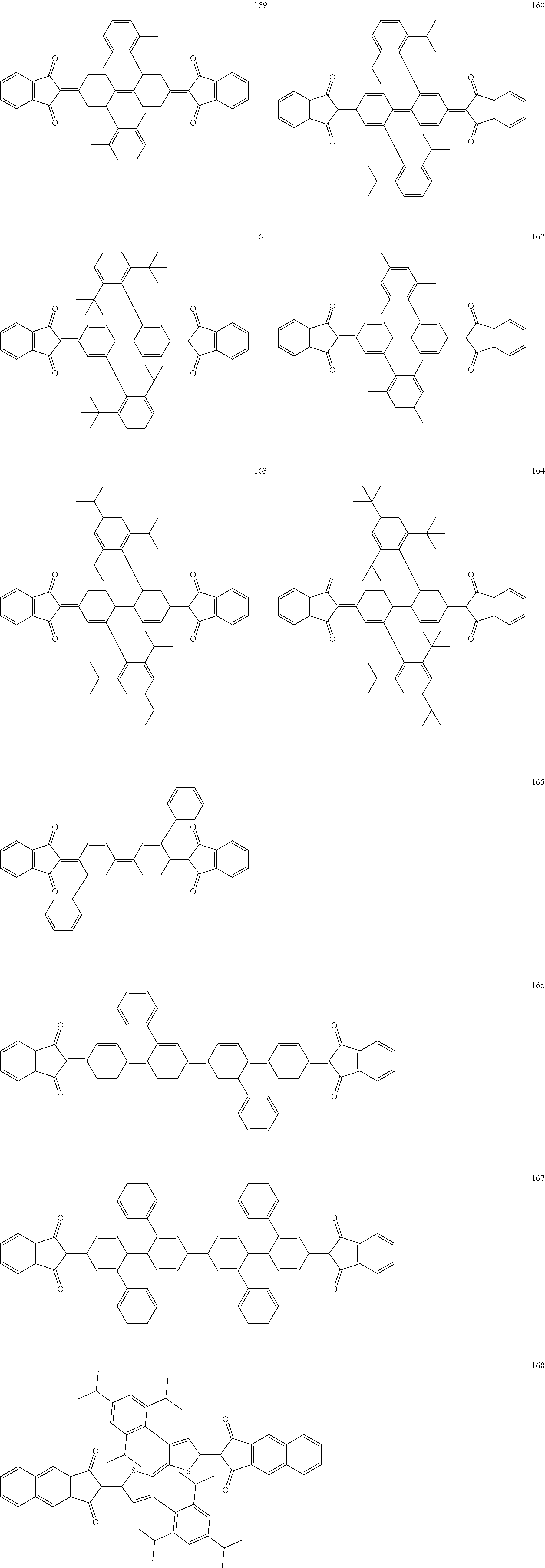

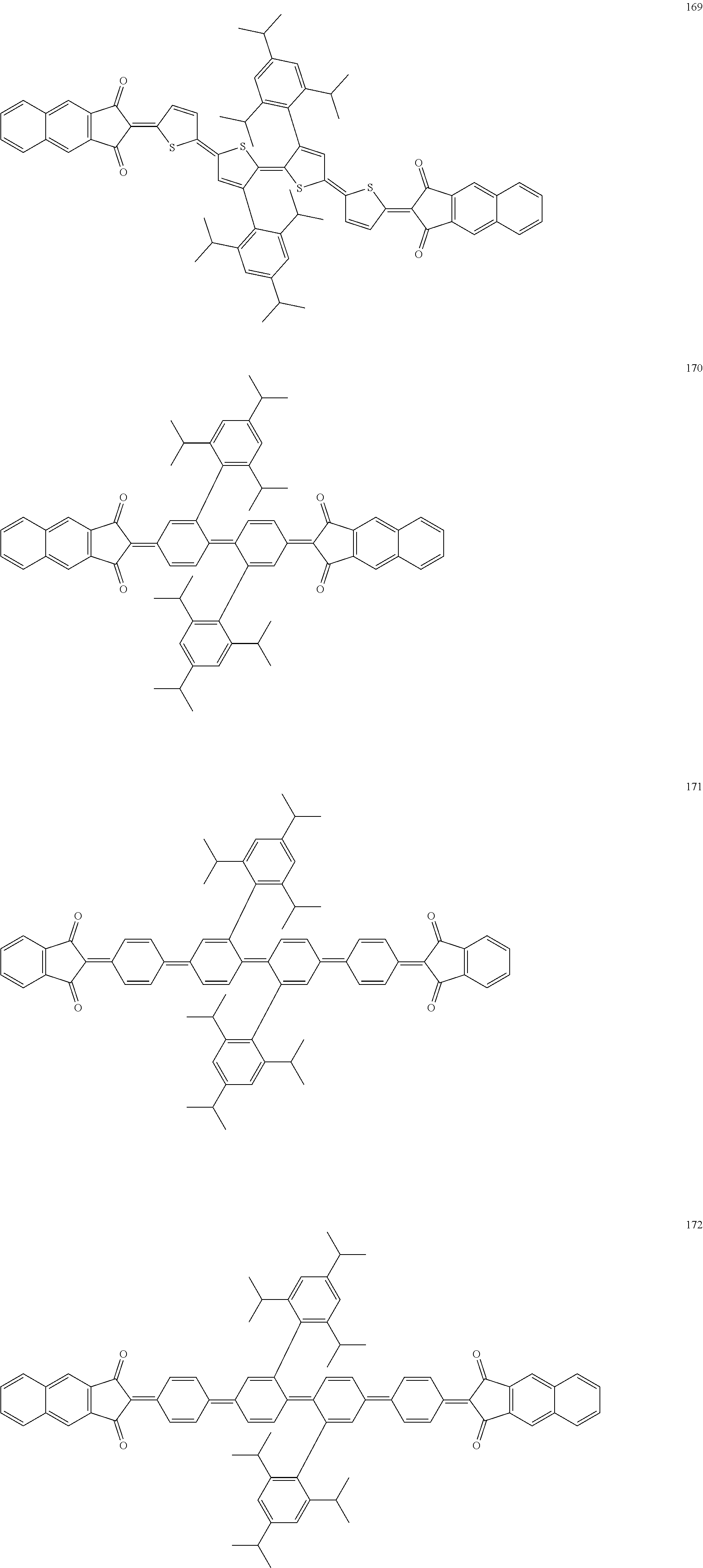

14. An organic photodetector comprising:
a first electrode;
a second electrode facing the first electrode; and
an active layer between the first electrode and the second electrode,
wherein the active layer comprises the compound represented by one selected from Formulae 1 to 4 of claim 1.

15. The organic photodetector of claim 14, wherein the active layer absorbs green, red, and/or near-infrared rays.

16. The organic photodetector of claim 14, wherein the active layer comprises the compound represented by one selected from Formulae 1 to 4 as an electron acceptor and/or an electron donor.

17. The organic photodetector of claim 14, wherein the first electrode is an anode,
the second electrode is a cathode,
the organic photodetector further comprises a hole transport region between the active layer and the first electrode, and
the hole transport region comprises a hole injection layer, a hole transport layer, an auxiliary layer, an electron-blocking layer, or any combination thereof.

18. The organic photodetector of claim 14, wherein the first electrode is an anode,
the second electrode is a cathode,
the organic photodetector further comprises an electron transport region between the active layer and the second electrode, and
the electron transport region comprises a buffer layer, a hole-blocking layer, an electron transport layer, an electron injection layer, or any combination thereof.

19. An electronic apparatus comprising the organic photodetector of claim 14.

20. The electronic apparatus of claim 19, further comprising a light-emitting device.
